(19) United States
(12) Patent Application Publication
(10) Pub. No.: US 2018/0267043 A1
(43) Pub. Date: Sep. 20, 2018

(54) USE OF BIOMARKERS FOR PREDICTING CLINICAL SENSITIVITY TO CANCER TREATMENT

(71) Applicant: CELGENE CORPORATION, Summit, NJ (US)

(72) Inventors: Antonia LOPEZ-GIRONA, San Diego, CA (US); Brian E. CATHERS, San Diego, CA (US); Gang LU, San Diego, CA (US); Pilgrim JACKSON, San Diego, CA (US); Hiroshi HANDA, Tokyo (JP)

(21) Appl. No.: 15/517,445

(22) PCT Filed: Oct. 6, 2015
(86) PCT No.: PCT/US15/54227

§ 371 Date: Apr. 6, 2017

Related U.S. Application Data
(60) Provisional application No. 62/061,050, filed on Oct. 7, 2014.
(60) Provisional application No. 62/087,111, filed on Dec. 3, 2014.

(51) Int. Cl. G01N 33/574

(52) U.S. Cl. CPC G01N 33/57426; G01N 33/57484; A61K 31/454; G01N 2800/60; G01N 2800/56; G01N 2800/52

(57) ABSTRACT

A method of identifying a subject having cancer who is likely to be responsive to a treatment compound, comprising administering the treatment compound to a subject having cancer; obtaining a sample from the subject; determining the level of a biomarker in the sample from the subject; and diagnosing the subject as being likely to be responsive to the treatment compound if the level of the biomarker in the sample of the subject changes as compared to a reference level of the biomarker; wherein the treatment compound is a compound of Formula (I):

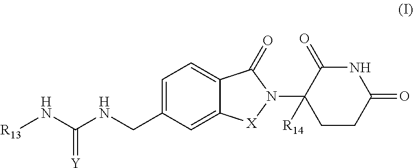

### 1. FIELD

Provided herein, in some embodiments, are methods of using certain biomarkers, such as eRF3a, eRF3b, eRF3c, ATF4, ATF3, or DDIT3, in predicting and monitoring clinical sensitivity and therapeutic response to certain compounds in patients having various diseases and disorders, such as cancer (e.g., lymphoma, multiple myeloma (MM), and leukemia such as acute myeloid leukemia (AML)). Further provided are kits for carrying out the methods. Also provided herein, in certain embodiments, are methods of determining the efficacy of a compound in treating diseases.

### 2. BACKGROUND

Cancer is characterized primarily by an increase in the number of abnormal cells derived from a given normal tissue, invasion of adjacent tissues by these abnormal cells, or lymphatic or blood-borne spread of malignant cells to regional lymph nodes and to distant sites (metastasis). In general, cancer is divided into solid cancer and blood borne cancer. Examples of solid cancer include, but are not limited to, melanoma, adrenal carcinoma, breast carcinoma, renal cell cancer, pancreatic carcinoma, and small-cell lung carcinoma (SCLC), etc.

Blood cancer generally includes three main types: lymphoma, leukemia, and myeloma. Lymphoma refers to cancers that originate in the lymphatic system. Lymphoma includes, but are not limited to, Hodgkin's lymphoma, non-Hodgkin's lymphoma (NHL), diffuse large B-cell lymphoma (DLBCL), and peripheral T-cell lymphomas (PTCL), etc. Leukemia refers to malignant neoplasms of the blood-forming tissues. Acute leukemia involves predominantly undifferentiated cell populations, whereas chronic leukemia involves more mature cell forms. Acute leukemia is divided into acute lymphoblastic leukemia (ALL) and acute myeloblastic leukemia (AML) types. The Merck Manual, 946-949 (17th ed. 1999). Chronic leukemia is divided into chronic lymphocytic leukemia (CLL) or chronic myelocytic leukemia (CML). The Merck Manual, 949-952 (17th ed. 1999). Myeloma is a cancer of plasma cells in the bone marrow. Because myeloma frequently occurs at many sites in the bone marrow, it is often referred to as multiple myeloma (MM).

Current cancer therapy may involve surgery, chemotherapy, hormonal therapy and/or radiation treatment to eradicate neoplastic cells in a patient (see, e.g., Stockdale, Medicine, vol. 3, Chapter 12, Section IV (Rubenstein and Federman eds., 1998). Recently, cancer therapy could also involve biological therapy or immunotherapy. All of these approaches may pose significant drawbacks for the patient.

A tremendous demand therefore exists for new methods, treatments and compositions that can be used to treat patients with cancer including but not limited to, lymphoma (e.g., NHL), MM, leukemia (e.g., AML), and solid cancer.

A number of studies have been conducted with the aim of providing compounds that can safely and effectively be used to treat cancers. Clinical efficacy of these compounds cannot easily be correctly predicted, as it can only be measured in terms of patient response, which usually requires a minimum of several months of treatment. In view of the deficiencies of the conventional methods, there is a need to develop efficient, sensitive, and accurate methods to detect, quantify, and characterize the pharmacodynamic activity of certain compounds. The present invention satisfies these and other needs.

### 3. SUMMARY OF THE INVENTION

In one aspect, provided herein is a method of identifying a subject having cancer who is likely to be responsive to a treatment compound, comprising:

(a) administering the treatment compound to the subject having the cancer;

(b) obtaining a sample from the subject;

(c) determining the level of a biomarker in the sample from the subject; and

(d) diagnosing the subject as being likely to be responsive to the treatment compound if the level of the biomarker in the sample of the subject changes as compared to a reference level of the biomarker;

wherein the treatment compound is a compound of Formula I:

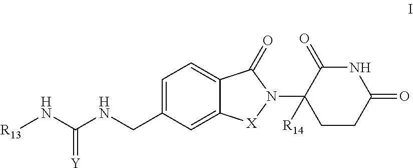

or a pharmaceutically acceptable salt, solvate, stereoisomer, isotopologue, prodrug, hydrate, co-crystal, clathrate, or a polymorph thereof, wherein:

X is CH₂ or C═O;

Y is O or S;

R¹³ is: (C₁-C₁₀)alkyl; (C₁-C₁₀)alkoxy; or 5 to 10 membered aryl or heteroaryl, optionally substituted with one or more of:

halogen; cyano; (C₁-C₆)alkylenedioxy; (C₁-C₆)alkoxy, itself optionally substituted with one or more halogen; (C₁-C₆)alkyl, itself optionally substituted with one or more halogen; or (C₁-C₆)alkylthio, itself optionally substituted with one or more halogen; and

R¹⁴ is H or (C₁-C₆)alkyl.

Provided herein is a method of identifying a subject having cancer who is likely to be responsive to a treatment compound, comprising:

(a) obtaining a sample from the subject having the cancer;

(b) administering the treatment compound to the sample from the subject;

(c) determining the level of a biomarker in the sample from the subject; and

(d) diagnosing the subject as being likely to be responsive to the treatment compound if the level of the biomarker in the sample of the subject changes as compared to a reference level of the biomarker;

wherein the treatment compound is a compound of Formula I:

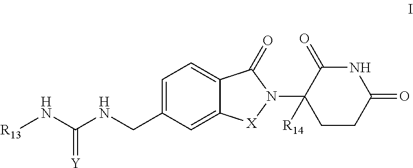

or a pharmaceutically acceptable salt, solvate, stereoisomer, isotopologue, prodrug, hydrate, co-crystal, clathrate, or a polymorph thereof, wherein:

X is CH₂ or C═O;

Y is O or S;

R¹³ is: (C₁-C₁₀)alkyl; (C₁-C₁₀)alkoxy; or 5 to 10 membered aryl or heteroaryl, optionally substituted with one or more of:

halogen; cyano; (C₁-C₆)alkylenedioxy; (C₁-C₆)alkoxy, itself optionally substituted with one or more halogen; (C₁-C₆)alkyl, itself optionally substituted with one or more halogen; or (C₁-C₆)alkylthio, itself optionally substituted with one or more halogen; and

R¹⁴ is H or (C₁-C₆)alkyl.

In certain embodiments, the change in the level of the biomarker in the sample of the subject is an increase compared to the reference level of the biomarker.

In certain embodiments, the change in the level of the biomarker in the sample of the subject is a decrease compared to the reference level of the biomarker.

Also provided herein is a method of treating cancer, comprising:

(a) obtaining a sample from a subject having the cancer;

(b) determining the level of a biomarker in the sample from the subject;

(c) diagnosing the subject as being likely to be responsive to a treatment compound if the level of the biomarker in the sample of the subject changes as compared to a reference level of the biomarker; and

(d) administering a therapeutically effective amount of the treatment compound to the subject diagnosed as being likely to be responsive to the treatment compound;

wherein the treatment compound is a compound of Formula I:

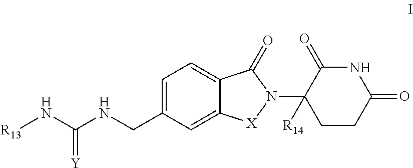

or a pharmaceutically acceptable salt, solvate, stereoisomer, isotopologue, prodrug, hydrate, co-crystal, clathrate, or a polymorph thereof, wherein:

X is CH₂ or C═O;

Y is O or S;

R¹³ is: (C₁-C₁₀)alkyl; (C₁-C₁₀)alkoxy; or 5 to 10 membered aryl or heteroaryl, optionally substituted with one or more of:

halogen; cyano; (C₁-C₆)alkylenedioxy; (C₁-C₆)alkoxy, itself optionally substituted with one or more halogen; (C₁-C₆)alkyl, itself optionally substituted with one or more halogen; or (C₁-C₆)alkylthio, itself optionally substituted with one or more halogen; and

R¹⁴ is H or (C₁-C₆)alkyl.

In certain embodiments, the change in the level of the biomarker in the sample of the subject is an increase compared to the reference level of the biomarker.

In certain embodiments, the change in the level of the biomarker in the sample of the subject is a decrease compared to the reference level of the biomarker.

Also provided herein is a method of predicting the responsiveness of a subject having or suspected of having cancer to a treatment compound, comprising:

(a) administering the treatment compound to the subject having the cancer;

(b) obtaining a sample from the subject;

(c) determining the level of a biomarker in the sample from the subject;

(d) predicting the subject as being likely to be responsive to a treatment of the cancer with the treatment compound if the level of the biomarker in the sample changes as compared to the level of the biomarker obtained from a reference sample;

wherein the treatment compound is a compound of Formula I:

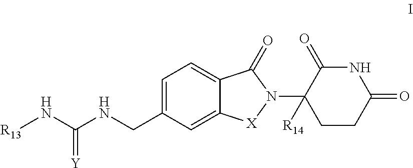

or a pharmaceutically acceptable salt, solvate, stereoisomer, isotopologue, prodrug, hydrate, co-crystal, clathrate, or a polymorph thereof, wherein:

X is CH₂ or C═O;

Y is O or S;

R¹³ is: (C₁-C₁₀)alkyl; (C₁-C₁₀)alkoxy; or 5 to 10 membered aryl or heteroaryl, optionally substituted with one or more of:

halogen; cyano; (C₁-C₆)alkylenedioxy; (C₁-C₆)alkoxy, itself optionally substituted with one or more halogen; (C₁-C₆)alkyl, itself optionally substituted with one or more halogen; or (C₁-C₆)alkylthio, itself optionally substituted with one or more halogen; and

R¹⁴ is H or (C₁-C₆)alkyl.

Provided herein is a method of predicting the responsiveness of a subject having or suspected of having cancer to a treatment compound, comprising:

(a) obtaining a sample from the subject having the cancer;

(b) administering the treatment compound to the sample from the subject;

(c) determining the level of a biomarker in the sample from the subject;

(d) predicting the subject as being likely to be responsive to a treatment of the cancer with the treatment compound if the level of the biomarker in the sample changes as compared to the level of the biomarker obtained from a reference sample;

wherein the treatment compound is a compound of Formula I:

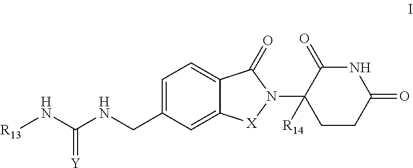

or a pharmaceutically acceptable salt, solvate, stereoisomer, isotopologue, prodrug, hydrate, co-crystal, clathrate, or a polymorph thereof, wherein:

X is CH₂ or C═O;

Y is O or S;

R¹³ is: (C₁-C₁₀)alkyl; (C₁-C₁₀)alkoxy; or 5 to 10 membered aryl or heteroaryl, optionally substituted with one or more of:

halogen; cyano; (C₁-C₆)alkylenedioxy; (C₁-C₆)alkoxy, itself optionally substituted with one or more halogen; (C₁-C₆)alkyl, itself optionally substituted with one or more halogen; or (C₁-C₆)alkylthio, itself optionally substituted with one or more halogen; and

R¹⁴ is H or (C₁-C₆)alkyl.

In certain embodiments, the level of the biomarker in the sample is higher than the level of the biomarker obtained from the reference sample. In certain embodiments, the level of the biomarker in the sample is less than the level of the biomarker obtained from the reference sample.

Also provided herein is a method of monitoring the efficacy of a treatment compound in treating cancer in a subject, comprising:

(a) administering the treatment compound to the subject having the cancer;

(b) obtaining a sample from the subject having the cancer;

(c) determining the level of a biomarker in the sample from the subject;

(d) comparing the level of the biomarker in the sample with the level of the biomarker obtained from a reference sample, wherein a change in the level as compared to the reference is indicative of the efficacy of the treatment compound in treating cancer in the subject;

wherein the treatment compound is a compound of Formula I:

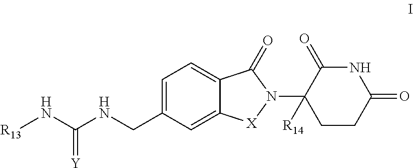

or a pharmaceutically acceptable salt, solvate, stereoisomer, isotopologue, prodrug, hydrate, co-crystal, clathrate, or a polymorph thereof, wherein:

X is CH₂ or C═O;

Y is O or S;

R¹³ is: (C₁-C₁₀)alkyl; (C₁-C₁₀)alkoxy; or 5 to 10 membered aryl or heteroaryl, optionally substituted with one or more of:

halogen; cyano; (C₁-C₆)alkylenedioxy; (C₁-C₆)alkoxy, itself optionally substituted with one or more halogen; (C₁-C₆)alkyl, itself optionally substituted with one or more halogen; or (C₁-C₆)alkylthio, itself optionally substituted with one or more halogen; and

R¹⁴ is H or (C₁-C₆)alkyl.

In certain embodiments, an increased level as compared to the reference is indicative of the efficacy of the treatment compound in treating cancer in the subject. In certain embodiments, a decreased level as compared to the reference is indicative of the efficacy of the treatment compound in treating cancer in the subject.

Also provided herein is a method of treating cancer, further comprising administering a therapeutically effective amount of a second active agent or a support care therapy.

In certain embodiments, the second active agent is a hematopoietic growth factor, cytokine, anti-cancer agent, antibiotic, cox-2 inhibitor, immunomodulatory agent, immunosuppressive agent, corticosteroid, therapeutic antibody that specifically binds to a cancer antigen or a pharmacologically active mutant, or derivative thereof.

In some embodiments of the various methods provided herein, the reference is prepared by using a control sample obtained from the subject prior to administering the treatment compound to the subject; and wherein the control sample is from the same source as the sample.

In some embodiments of the various methods provided herein, the reference is prepared by using a control sample obtained from a healthy subject not having cancer; and wherein the control sample is from the same source as the sample.

In some embodiments of the various methods provided herein, the cancer is MM, lymphoma, or leukemia. In some embodiments of the various methods provided herein, the cancer is lymphoma. In some embodiments of the various methods provided herein, the cancer is leukemia. In certain embodiments, the leukemia is CLL, CML, ALL, or AML. In yet other embodiments, the leukemia is AML.

In some embodiments of the various methods provided herein for leukemia, the leukemia is relapsed, refractory, or resistant to conventional therapy.

In some embodiments of the methods provided herein, the biomarker is a protein that is directly or indirectly affected by CRBN. In certain embodiments, the biomarker is a protein that is directly affected by CRBN (such as a CRBN-associated protein). In other embodiments, the biomarker is a protein that is indirectly affected by CRBN (such as a downstream protein that is affected by signaling pathways). In some embodiments of the various methods provided herein, the biomarker is a CRBN-associated protein (CAP). In some embodiments, the CAP is a substrate of CRBN. In some embodiments, the CAP is a binding partner of CRBN under certain conditions. In some embodiments, the CAP is a downstream factor impacted by the substrate of CRBN.

In some embodiments, the biomarker has a function in unfolded protein response (UPR). In some embodiments, the biomarker has a function in PERK related signaling pathway. In some embodiments, the biomarker has a function in XBP1 related signaling pathway. In some embodiments, the biomarker has a function in ATF6 related signaling pathway.

In some embodiments, the biomarker is an eRF3 family member selected from the group consisting of eRF3a, eRF3b, and eRF3c. In some embodiments, the biomarker is eRF3a, eRF3b, or eRF3c, and wherein the level of the biomarker decreases as compared to a reference. In one embodiment, biomarker is eRF3a. In another embodiment, the biomarker is eRF3b. In yet another embodiment, the biomarker is eRF3c.

In some embodiments, the biomarker is selected from the group consisting of ATF4, ATF3 and DDIT3, and wherein the level of the biomarker increases as compared to a reference. In one embodiment, the biomarker is ATF4. In another embodiment, the biomarker is ATF3. In yet another embodiment, the biomarker is DDIT3.

In some embodiments of the various methods provided herein, the level of the biomarkers is measured by determining the protein level of the biomarkers.

In other embodiments of the various methods provided herein, the method provided herein further comprises contacting proteins within the sample with a first antibody that immunospecifically binds to the biomarker protein.

In one embodiment, the method provided herein further comprises:

(i) contacting the biomarker protein bound to the first antibody with a second antibody with a detectable label, wherein the second antibody immunospecifically binds to the biomarker protein, and wherein the second antibody immunospecifically binds to a different epitope on the biomarker protein than the first antibody;

(ii) detecting the presence of the second antibody bound to the biomarker protein; and

(iii) determining the amount of the biomarker protein based on the amount of detectable label in the second antibody.

In another embodiment, the method provided herein further comprises:

(i) contacting the biomarker protein bound to the first antibody with a second antibody with a detectable label, wherein the second antibody immunospecifically binds to the first antibody;

(ii) detecting the presence of the second antibody bound to the first antibody; and

(iii) determining the amount of the biomarker protein based on the amount of detectable label in the second antibody.

In some embodiments of the various methods provided herein, the level of the biomarkers is measured by determining the mRNA level of the biomarkers. In other embodiments of the various methods provided herein, the level of the biomarkers is measured by determining the cDNA level of the biomarkers.

In some embodiments of the various methods provided herein, the treatment compound is a compound of Formula I:

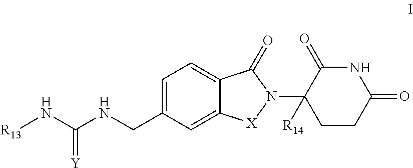

or a pharmaceutically acceptable salt, solvate, stereoisomer, isotopologue, prodrug, hydrate, co-crystal, clathrate, or a polymorph thereof, wherein:

X is CH₂;

Y is O;

R¹³ is 5 to 10 membered aryl or heteroaryl, optionally substituted with one or more of: halogen; cyano; (C₁-C₆)alkylenedioxy; (C₁-C₆)alkoxy, itself optionally substituted with one or more halogen; (C₁-C₆)alkyl, itself optionally substituted with one or more halogen; or (C₁-C₆)alkylthio, itself optionally substituted with one or more halogen; and

R¹⁴ is H.

In some embodiments of the various methods provided herein, the treatment compound is 1-(3-chloro-4-methylphenyl)-3-((2-(2,6-dioxopiperidin-3-yl)-1-oxoisoindolin-5-yl)methyl)urea

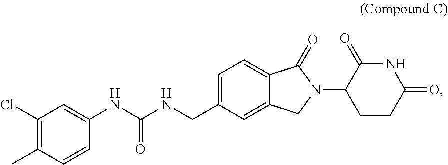 [SMILES: Cc1ccc(NC(=O)NCc2ccc3c(c2)CN(C2CCC(=O)NC2=O)C3=O)cc1Cl]

or a pharmaceutically acceptable salt, solvate, stereoisomer, isotopologue, prodrug, hydrate, co-crystal, clathrate, or a polymorph thereof.

### 4. BRIEF DESCRIPTION OF THE FIGURES

FIG. 1 shows identification of novel binding partners of CRBN induced by Compound C binding. The left part of FIG. 1 shows the silver staining gel of FLAG-HA CRBN immunoprecipitates, which was then analyzed by mass spectrometry to identify novel CRBN-binding proteins. Arrows point to the expected positions of DDB1, GSPT1, PABP1, and CRBN. The right part of FIG. 1 confirms the novel CRBN-binding proteins induced by binding of Compound C, including GSPT1/eRF3a, GSPT2/eRF3b, and HBS1L/eRF3c. The right part of FIG. 1 further demonstrates that increased concentration of Compound C induces degradation of GSPT1, GSPT2, and HBS1L.

FIG. 2 shows that Compound C promotes the interaction between CRBN and its substrates IKZF1 or GSPT1/2 in vitro, and that lenalidomide promotes the binding of CRBN with its substrate IKZF1, but not with substrates GSPT1 or GSPT2. FIG. 2 also shows that the lenalidomide-induced CRBN-IKZF1 interaction is abolished by a specific mutation Q146H in IKZF1.

FIG. 3 shows that the levels of Aiolos, CK1a, and GSPT1 in the lymphoma cell line OCI-LY10 are reduced in response to treatment with Compound C, using Western blot analysis.

FIG. 4 shows that Compound C induces depletion of GSPT1 and its binding partner eRF1 in 293FT HEK cells. FIG. 4 shows that Compound C induces degradation of GSPT1 and eRF1, and that overexpression of GSPT1 reduced this degradation effect. FIG. 4 also shows that introduction of CRBN isoforms 2 (CRBNiso2) or CRBNiso2 W385A mutant into the CRBN−/− cells restored Compound C-induced degradation of GSPT1 and eRF1, suggesting Compound C-induced degradation of GSPT1 and eRF1 is CRBN-dependent.

FIG. 5 shows the identification of specific amino acids in human CRBN that are essential for the destruction of IKZF1/3 or GSPT1/2. FIG. 5 shows that the specific mutation E376V in human CRBNiso2 abolished Compound C-induced degradation of GSPT1/2 but not Compound C-induced degradation of IKZF1/3, suggesting the essential role of E376 in CRBN for the destruction of GSPT1/2. FIG. 5 also shows that the specific mutation V387I in human CRBNiso2 abolished Compound C-induced degradation of IKZF1/3 but not Compound C-induced degradation of GSPT1/2, suggesting the essential role of V387 in CRBN for the destruction of IKZF1/3.

FIG. 6 shows that the V380E and I391V mutations are sufficient to reactivate mouse CRBN to trigger the degradation of IKZF1/3 and GSPT1/2, respectively. FIG. 6 shows that theV380E mutation in mouse CRBN isoform 2 restored Compound C-induced degradation of GSPT1/2, whereas the I391V mutation in mouse CRBN isoform 2 restored both lenalidomide- and Compound C-induced degradation of IKZF1/3.

FIG. 7 shows that overexpression of GSPT1 conferred Compound C resistance to HEK 293FT Cells. The left panel of FIG. 7 shows that Compound C induced growth inhibition, but overexpression of GSPT1 created resistance to Compound C-induced growth inhibition. The right panel of FIG. 7 shows that Compound C induced degradation of GSPT1, and that CMV promoter conferred the highest overexpression level of GSPT1, followed by EFla and UbcP promoters. The left and right panels of FIG. 7 demonstrate the correlation between overexpression of GSPT1 and cell resistance to Compound C-induced growth inhibition.

FIG. 8 shows that 293FT human embryonic kidney cells expressing GSPT1-specific shRNAs (such as shGSPT1-1, shGSPT1-2, shGSPT1-3, and shGSPT1-4) exhibited various degrees of inhibition on cell proliferation, and that GSPT1 depletion using shGSPT1-4 also reduced the levels of eRF1 and CRBN.

FIGS. 9A-9B show that loss of GSPT1 made HEK 293FT cells susceptible to Compound C-induced anti-proliferation. FIG. 9A shows that Compound C induced growth inhibition, and that depletion of GSPT1 increased sensitivity to Compound C-induced growth inhibition in HEK 293FT cells. FIG. 9B shows that the GSPT1-specific shRNA reduced the expression of GSPT1, and that Compound C induced degradation of GSPT1 and eRF1.

FIGS. 10A-10B show that depletion of GSPT1 sensitized MM cell lines to Compound C-induced growth inhibition. FIG. 10A shows that Compound C exhibited increased anti-proliferative effect in cells expressing shGSPT1-1 or shGSPT1-3. FIG. 10B shows that this increased sensitivity to Compound C-induced growth inhibition was likely due to depletion of GSPT1 and eRF1.

FIG. 11 shows that Compound C inhibits cell proliferation. FIG. 11 also shows that the anti-proliferative effect was abolished by depletion of CRBN using CRISPR genome editing tool and was dramatically reduced by overexpression of exogenous GSPT1 via the EFla promoter, in the human histiocytic lymphoma cell line U937 and the acute myeloid leukemia cell line Molm-13.

FIGS. 12A-12B show that depletion of GSPT1 sensitized the Human Acute Myeloblastic Leukemia Cell Line KG1 and the Acute Myelogenous Leukemia (AML3) cell lines to Compound C. FIG. 12A shows that Compound C exhibited anti-proliferative effect, and that the anti-proliferative effect increased when the expression of GSPT1 was downregulated by shGSPT1-1 or shGSPT1-3. FIG. 12B shows that both shGSPT1-1 and shGSPT1-3 reduced the expression of GSPT1 and eRF1.

FIG. 13 shows that Compound C induced the activation of the PERK branch of unfolded protein response (UPR) in 293FT HEK cells by inducing mRNA expression of components along the PERK pathway (such as ATF4, ATF3, DDIT3, PPP1R15A, and GADD45A). FIG. 13 also shows that this induction effect increased in cells with GSPT1 knockdown.

FIG. 14 shows that Compound C activated the XBP1 and ATF6 pathways in 293FT HEK cells by inducing mRNA expression of components along the XBP1 pathway (such as SEC24D, DNAJB9, DNAJC6, XBP1, EDEM1, EDEM2, and HYOU1) and components along the ATF6 pathway (such as XBP1, EDEM1, EDEM2, HYOU1, and HSPA5). FIG. 14 also shows that this induction effect increased in cells with GSPT1 knockdown.

FIGS. 15A-15B show that degradation of GSPT1 led to a loss of BIP immunoreactivity and ER stress but not acute apoptotic cell death in 293FT HEK cells. FIG. 15A shows that Compound C induced degradation of GSPT1. FIG. 15B shows that 20-hour treatment of Compound C did not affect cellular components in acute apoptotic cell death.

FIGS. 16A-16C show that Compound C-induced UPR preceded apoptotic cell death in DF15 cells. FIG. 16A shows that Compound C induced degradation of GSPT1, IKZF1, and IKZF3. FIG. 16B shows that Compound C increased the protein level of pEIF2α, ATF4, ATF3, DDIT3, cleaved Caspase-3, and cleaved PARP. FIG. 16C shows Compound C-induced mRNA expression of ATF4, ATF3, DDIT3, PPP1R15A, and GADD45A, components along the PERK/EIF2α/ATF4 pathway.

FIG. 17 shows that Compound C activated the XBP1 and ATF6 pathways in DF15 MM cells, and that Compound C induced mRNA expression of components along the XBP1 pathway (such as SEC24D, DNAJB9, XBP1, EDEM1, and HYOU1) and components along the ATF6 pathway (such as XBP1, EDEM1, HYOU1, and HSPA5).

FIGS. 18A-18C show that Compound C-induced UPR preceded apoptotic cell death in Human Acute Myeloblastic Leukemia Cell Line KG1. FIG. 18A shows that Compound C induced degradation of GSPT1, and that the protein levels of pEIF2α, ATF4, ATF3, and CHOP (DDIT3) increased in response to Compound C treatment. FIG. 18B shows that the levels of cleaved Caspase-8, BID, cleaved Caspase-9, cleaved Caspase-3, cleaved Caspase-7, and cleaved PARP increased in response to Compound C treatment, and that the levels of Mcl-1 and pS112-BAD decreased in response to Compound C treatment. FIG. 18C shows Compound C-induced mRNA levels of ATF4, ATF3, DDIT3, PPP1R15A, GADD45A, TNFRSF1B, and TNFRSF10B, components along the PERK/EIF2α/ATF4 pathway in KG1 cells.

FIG. 19 shows that Compound C induced UPR in Human Acute Myeloblastic Leukemia Cell Line KG1, and that Compound C induced mRNA expression of components along the XBP1 pathway (such as SEC24D, DNAJB9, EDEM1, and XBP1) and components along the ATF6 pathway (such as XBP1).

FIG. 20 shows the response to Compound C treatment in normal peripheral blood mononuclear cell (PBMC). FIG. 20 shows that Compound C decreased the expression of GSPT1, but increased the level of p-EIF2α, ATF3 (likely in a splicing variant) and DDIT3, which consequently activated Caspase-3 by increasing cleaved Caspase-3. The cleaved Caspase-3 then inactivated PARP by cleaving PARP and induced apoptosis.

FIG. 21 shows the prediction of sensitivity and resistance to Compound C in different cancer cell lines. FIG. 21 shows that Compound C-induced ER stress preceded Compound C-induced apoptosis. Whereas RPMI-8226 cells were resistant to Compound C-induced ER stress and apoptosis, KG1, DF15, AML3, and 293FT cells exhibited different levels of sensitivity to Compound C.

### 5. DETAILED DESCRIPTION OF THE INVENTION

The methods provided herein are based, in part, on the discovery that a changed level, e.g., an increased level and/or a decreased level, of certain molecules (e.g., mRNAs, cDNAs, or proteins) in a biological sample can be used as a biomarker to predict responsiveness of a subject having or suspected to have cancer (e.g., lymphoma, MM, or leukemia) to a treatment compound (e.g., Compound C, a pharmaceutically acceptable salt, solvate, stereoisomer, isotopologue, prodrug, hydrate, co-crystal, clathrate, or a polymorph thereof).

### 5.1 Definitions

As used herein, the term “cancer” includes, but is not limited to, solid cancer and blood born cancer. The term “cancer” refers to disease of tissues or organs, including but not limited to, cancers of the bladder, bone, blood, brain, breast, cervix, chest, colon, endrometrium, esophagus, eye, head, kidney, liver, lymph nodes, lung, mouth, neck, ovaries, pancreas, prostate, rectum, skin, stomach, testis, throat, and uterus. Specific cancers include, but are not limited to, advanced malignancy, amyloidosis, neuroblastoma, meningioma, hemangiopericytoma, multiple brain metastase, glioblastoma multiforms, glioblastoma, brain stem glioma, poor prognosis malignant brain tumor, malignant glioma, recurrent malignant giolma, anaplastic astrocytoma, anaplastic oligodendroglioma, neuroendocrine tumor, rectal adenocarcinoma, Dukes C & D colorectal cancer, unresectable colorectal carcinoma, metastatic hepatocellular carcinoma, Kaposi's sarcoma, karotype acute myeloblastic leukemia, Hodgkin's lymphoma, non-Hodgkin's lymphoma, cutaneous T-Cell lymphoma, cutaneous B-Cell lymphoma, diffuse large B-Cell lymphoma, low grade follicular lymphoma, malignant melanoma, malignant mesothelioma, malignant pleural effusion mesothelioma syndrome, peritoneal carcinoma, papillary serous carcinoma, gynecologic sarcoma, soft tissue sarcoma, scleroderma, cutaneous vasculitis, Langerhans cell histiocytosis, leiomyosarcoma, fibrodysplasia ossificans progressive, hormone refractory prostate cancer, resected high-risk soft tissue sarcoma, unrescectable hepatocellular carcinoma, Waldenstrom's macroglobulinemia, smoldering myeloma, indolent myeloma, fallopian tube cancer, androgen independent prostate cancer, androgen dependent stage IV non-metastatic prostate cancer, hormone-insensitive prostate cancer, chemotherapy-insensitive prostate cancer, papillary thyroid carcinoma, follicular thyroid carcinoma, medullary thyroid carcinoma, and leiomyoma.

As used herein, and unless otherwise specified, the terms “treat,” “treating,” and “treatment” refer to an action that occurs while a patient is suffering from the specified cancer, which reduces the severity of the cancer or retards or slows the progression of the cancer.

The term “sensitivity” or “sensitive” when made in reference to treatment with compound is a relative term which refers to the degree of effectiveness of the compound in lessening or decreasing the progress of a tumor or the disease being treated. For example, the term “increased sensitivity” when used in reference to treatment of a cell or tumor in connection with a compound refers to an increase of, at least about 5%, or more, in the effectiveness of the tumor treatment.

As used herein, the terms “compound” and “treatment compound” are used interchangeably, and include the compounds of Formula I. Non-limiting examples of compounds include those disclosed in Section 5.7 below.

As used herein, and unless otherwise specified, the term “therapeutically effective amount” of a compound is an amount sufficient to provide a therapeutic benefit in the treatment or management of a cancer, or to delay or minimize one or more symptoms associated with the presence of the cancer. A therapeutically effective amount of a compound means an amount of therapeutic agent, alone or in combination with other therapies, which provides a therapeutic benefit in the treatment or management of the cancer. The term “therapeutically effective amount” can encompass an amount that improves overall therapy, reduces or avoids symptoms or causes of cancer, or enhances the therapeutic efficacy of another therapeutic agent. The term also refers to the amount of a compound that is sufficient to elicit the biological or medical response of a biological molecule (e.g., a protein, enzyme, RNA, or DNA), cell, tissue, system, animal, or human, which is being sought by a researcher, veterinarian, medical doctor, or clinician.

The term “responsiveness” or “responsive” when used in reference to a treatment refers to the degree of effectiveness of the treatment in lessening or decreasing the symptoms of a disease, e.g., MM or AML, being treated. For example, the term “increased responsiveness” when used in reference to a treatment of a cell or a subject refers to an increase in the effectiveness in lessening or decreasing the symptoms of the disease when measured using any methods known in the art. In certain embodiments, the increase in the effectiveness is at least about 5%, at least about 10%, at least about 20%, at least about 30%, at least about 40%, or at least about 50%.

As used herein, the terms “effective subject response,” “effective patient response,” and “effective patient tumor response” refer to any increase in the therapeutic benefit to the patient. An “effective patient tumor response” can be, for example, about 5%, about 10%, about 25%, about 50%, or about 100% decrease in the rate of progress of the tumor. An “effective patient tumor response” can be, for example, about 5%, about 10%, about 25%, about 50%, or about 100% decrease in the physical symptoms of a cancer. An “effective patient tumor response” can also be, for example, about 5%, about 10%, about 25%, about 50%, about 100%, about 200%, or more increase in the response of the patient, as measured by any suitable means, such as gene expression, cell counts, assay results, tumor size, etc.

An improvement in the cancer or cancer-related disease can be characterized as a complete or partial response. “Complete response” refers to an absence of clinically detectable disease with normalization of any previously abnormal radiographic studies, bone marrow, and cerebrospinal fluid (CSF) or abnormal monoclonal protein measurements. “Partial response” refers to at least about 10%, about 20%, about 30%, about 40%, about 50%, about 60%, about 70%, about 80%, or about 90% decrease in all measurable tumor burden (i.e., the number of malignant cells present in the subject, or the measured bulk of tumor masses or the quantity of abnormal monoclonal protein) in the absence of new lesions. The term “treatment” contemplates both a complete and a partial response.

The term “likelihood” generally refers to an increase in the probability of an event. The term “likelihood” when used in reference to the effectiveness of a patient tumor response generally contemplates an increased probability that the rate of tumor progress or tumor cell growth will decrease. The term “likelihood” when used in reference to the effectiveness of a patient tumor response can also generally mean the increase of indicators, such as mRNA or protein expression, that may evidence an increase in the progress in treating the tumor.

The term “predict” generally means to determine or tell in advance. When used to “predict” the effectiveness of a cancer treatment, for example, the term “predict” can mean that the likelihood of the outcome of the cancer treatment can be determined at the outset, before the treatment has begun, or before the treatment period has progressed substantially.

The term “monitor,” as used herein, generally refers to the overseeing, supervision, regulation, watching, tracking, or surveillance of an activity. For example, the term “monitoring the effectiveness of a compound” refers to tracking the effectiveness in treating cancer in a patient or in a tumor cell culture. Similarly, the term “monitoring,” when used in connection with patient compliance, either individually, or in a clinical trial, refers to the tracking or confirming that the patient is actually taking a drug being tested as prescribed. The monitoring can be performed, for example, by following the expression of mRNA or protein biomarkers.

“Tumor,” as used herein, refers to all neoplastic cell growth and proliferation, whether malignant or benign, and all pre-cancerous and cancerous cells and tissues. “Neoplastic,” as used herein, refers to any form of dysregulated or unregulated cell growth, whether malignant or benign, resulting in abnormal tissue growth. Thus, “neoplastic cells” include malignant and benign cells having dysregulated or unregulated cell growth.

As used herein, the term “cereblon-associated protein” or “CAP” refers to a protein that interacts with or binds to CRBN directly or indirectly. For example, the term refers to any protein that directly binds to cereblon, as well as any protein that is an indirect downstream effector of cereblon pathways. In certain embodiments, a “cereblon-associated protein” or “CAP” is a substrate of CRBN, for example, a protein substrate of the E3 ubiquitin ligase complex involving CRBN, or the downstream substrates thereof. In some embodiments, a “cereblon-associated protein” or “CAP” is eRF3a, eRF3b, eRF3c, eRF1, IKZF1, IKZF2, or IKZF3.

The term “regulate” as used herein refers to controlling the activity of a molecule or biological function, such as enhancing or diminishing the activity or function.

The terms “cancer” and “cancerous” refer to or describe the physiological condition in mammals that is typically characterized by unregulated cell growth. Examples of cancer include, but are not limited to, blood-borne cancers (e.g., multiple myeloma, lymphoma and leukemia), and solid cancers.

The term “refractory” or “resistant” refers to a circumstance where patients, even after intensive treatment, have residual cancer cells (e.g., leukemia or lymphoma cells) in their lymphatic system, blood, and/or blood forming tissues (e.g., marrow).

A “biological marker” or “biomarker” is a substance whose detection indicates a particular biological state, such as, for example, the presence of cancer. In some embodiments, biomarkers can be determined individually. In other embodiments, several biomarkers can be measured simultaneously.

In some embodiments, a “biomarker” indicates a change in the level of mRNA expression that may correlate with the risk or progression of a disease, or with the susceptibility of the disease to a given treatment. In some embodiments, the biomarker is a nucleic acid, such as mRNA or cDNA.

In additional embodiments, a “biomarker” indicates a change in the level of polypeptide or protein expression that may correlate with the risk or progression of a disease, or patient's susceptibility to treatment. In some embodiments, the biomarker can be a polypeptide or protein, or a fragment thereof. The relative level of specific proteins can be determined by methods known in the art. For example, antibody based methods, such as an immunoblot, enzyme-linked immunosorbent assay (ELISA), or other methods can be used.

The terms “polypeptide” and “protein,” as used interchangeably herein, refer to a polymer of three or more amino acids in a serial array, linked through peptide bonds. The term “polypeptide” includes proteins, protein fragments, protein analogues, oligopeptides, and the like. The term “polypeptide” as used herein can also refer to a peptide. The amino acids making up the polypeptide may be naturally derived, or may be synthetic. The polypeptide can be purified from a biological sample. The polypeptide, protein, or peptide also encompasses modified polypeptides, proteins, and peptides, e.g., glycopolypeptides, glycoproteins, or glycopeptides; or lipopolypeptides, lipoproteins, or lipopeptides.

The term “antibody,” “immunoglobulin,” or “Ig” as used interchangeably herein, encompasses fully assembled antibodies and antibody fragments that retain the ability to specifically bind to the antigen. Antibodies provided herein include, but are not limited to, synthetic antibodies, monoclonal antibodies, polyclonal antibodies, recombinantly produced antibodies, multispecific antibodies (including bi-specific antibodies), human antibodies, humanized antibodies, chimeric antibodies, intrabodies, single-chain Fvs (scFv) (e.g., including monospecific, bispecific, etc.), camelized antibodies, Fab fragments, F(ab′) fragments, disulfide-linked Fvs (sdFv), anti-idiotypic (anti-Id) antibodies, and epitope-binding fragments of any of the above. In particular, antibodies provided herein include immunoglobulin molecules and immunologically active portions of immunoglobulin molecules, i.e., antigen binding domains or molecules that contain an antigen-binding site that immunospecifically binds to CRBN antigen (e.g., one or more complementarity determining regions (CDRs) of an anti-CRBN antibody). The antibodies provided herein can be of any class (e.g., IgG, IgE, IgM, IgD, and IgA) or any subclass (e.g., IgG1, IgG2, IgG3, IgG4, IgA1, and IgA2) of immunoglobulin molecule. In some embodiments, the anti-CRBN antibodies are fully human, such as fully human monoclonal CRBN antibodies. In certain embodiments, antibodies provided herein are IgG antibodies, or a subclass thereof (e.g., human IgG1 or IgG4).

The terms “antigen binding domain,” “antigen binding region,” “antigen binding fragment,” and similar terms refer to the portion of an antibody that comprises the amino acid residues that interact with an antigen and confer on the binding agent its specificity and affinity for the antigen (e.g., the CDR). The antigen binding region can be derived from any animal species, such as rodents (e.g., rabbit, rat, or hamster) and humans. In some embodiments, the antigen binding region is of human origin.

The term “constant region” or “constant domain” of an antibody refers to a carboxy terminal portion of the light and heavy chain that is not directly involved in binding of the antibody to antigen but exhibits various effector functions, such as interaction with the Fc receptor. The term refers to the portion of an immunoglobulin molecule that has a more conserved amino acid sequence relative to the other portion of the immunoglobulin, the variable domain, which contains the antigen binding site. The constant domain contains CH1, CH2 and CH3 domains of the heavy chain and the CL domain of the light chain.

The term “epitope” as used herein refers to a localized region on the surface of an antigen that is capable of binding to one or more antigen binding regions of an antibody, that has antigenic or immunogenic activity in an animal, such as a mammal (e.g., a human), and that is capable of eliciting an immune response. An epitope having immunogenic activity is a portion of a polypeptide that elicits an antibody response in an animal. An epitope having antigenic activity is a portion of a polypeptide to which an antibody immunospecifically binds as determined by any method well known in the art, for example, by the immunoassays described herein. Antigenic epitopes need not necessarily be immunogenic. Epitopes usually consist of chemically active surface groupings of molecules, such as amino acids or sugar side chains, and have specific three dimensional structural characteristics as well as specific charge characteristics. A region of a polypeptide contributing to an epitope may be contiguous amino acids of the polypeptide, or the epitope may come together from two or more non-contiguous regions of the polypeptide. The epitope may or may not be a three-dimensional surface feature of the antigen.

The terms “fully human antibody” and “human antibody” are used interchangeably herein and refer to an antibody that comprises a human variable region and, in some embodiments, a human constant region. In specific embodiments, the terms refer to an antibody that comprises a variable region and a constant region of human origin. The term “fully human antibody” includes antibodies having variable and constant regions corresponding to human germline immunoglobulin sequences as described by Kabat et al., Sequences of Proteins of Immunological Interest, U.S. Department of Health and Human Services, NIH Publication No. 91-3242 (5th ed. 1991).

The phrase “recombinant human antibody” includes human antibodies that are prepared, expressed, created, or isolated by recombinant means, such as antibodies expressed using a recombinant expression vector transfected into a host cell, antibodies isolated from a recombinant, combinatorial human antibody library, antibodies isolated from an animal (e.g., a mouse or a cow) that is transgenic and/or transchromosomal for human immunoglobulin genes (see, e.g., Taylor et al., Nucl. Acids Res. 1992, 20:6287-6295) or antibodies prepared, expressed, created, or isolated by any other means that involves splicing of human immunoglobulin gene sequences to other DNA sequences. Such recombinant human antibodies can have variable and constant regions derived from human germline immunoglobulin sequences. See Kabat et al., Sequences of Proteins of Immunological Interest, U.S. Department of Health and Human Services, NIH Publication No. 91-3242 (5th ed. 1991). In certain embodiments, however, such recombinant human antibodies are subjected to in vitro mutagenesis (or, when an animal transgenic for human Ig sequences is used, in vivo somatic mutagenesis) and thus the amino acid sequences of the heavy chain variable and light chain variable regions of the recombinant antibodies are sequences that, while derived from and related to human germline heavy chain variable and light chain variable sequences, may not naturally exist within the human antibody germline repertoire in vivo.

The term “heavy chain” when used in reference to an antibody refers to five distinct types, called alpha (α), delta (δ), epsilon (ε), gamma (γ) and mu (μ), based on the amino acid sequence of the heavy chain constant domain. These distinct types of heavy chains are well known and give rise to five classes of antibodies, IgA, IgD, IgE, IgG and IgM, respectively, including four subclasses of IgG, namely IgG1, IgG1, IgG3 and IgG4. In some embodiments the heavy chain is a human heavy chain.

The term “Kabat numbering” and similar terms are recognized in the art and refer to a system of numbering amino acid residues that are more variable (i.e., hypervariable) than other amino acid residues in the heavy and light chain variable regions of an antibody, or an antigen binding portion thereof. Kabat et al., Ann. NY Acad. Sci. 1971, 190:382-391; Kabat et al., Sequences of Proteins of Immunological Interest, U.S. Department of Health and Human Services, NIH Publication No. 91-3242 (5th ed. 1991). For the heavy chain variable region, the hypervariable region typically ranges from amino acid positions 31 to 35 for CDR1, amino acid positions 50 to 65 for CDR2, and amino acid positions 95 to 102 for CDR3. For the light chain variable region, the hypervariable region typically ranges from amino acid positions 24 to 34 for CDR1, amino acid positions 50 to 56 for CDR2, and amino acid positions 89 to 97 for CDR3. Other numbering schemes will be readily understood by those skilled in the art.

The term “light chain” when used in reference to an antibody refers to two distinct types, called kappa (κ) or lambda (λ) based on the amino acid sequence of the constant domains. Light chain amino acid sequences are well known in the art. In certain embodiments, the light chain is a human light chain.

The term “monoclonal antibody” refers to an antibody obtained from a population of homogenous or substantially homogeneous antibodies, and each monoclonal antibody will typically recognize a single epitope on the antigen. In some embodiments, a “monoclonal antibody,” as used herein, is an antibody produced by a single hybridoma or other cell, wherein the antibody immunospecifically binds to only an epitope as determined, e.g., by ELISA or other antigen-binding or competitive binding assay known in the art or in the Examples provided herein. The term “monoclonal” is not limited to any particular method for making the antibody. For example, monoclonal antibodies provided herein may be made by the hybridoma method as described in Kohler et al., Nature 1975, 256:495-497, or may be isolated from phage libraries using the techniques as described herein. Other methods for the preparation of clonal cell lines and of monoclonal antibodies expressed thereby are well known in the art. See, e.g., Short Protocols in Molecular Biology, Chapter 11 (Ausubel et al., eds., John Wiley and Sons, New York, 5th ed. 2002). Other exemplary methods of producing other monoclonal antibodies are provided in the Examples herein.

“Polyclonal antibodies” as used herein refers to an antibody population generated in an immunogenic response to a protein having many epitopes and thus includes a variety of different antibodies directed to the same or to different epitopes within the protein. Methods for producing polyclonal antibodies are known in the art. See, e.g., Short Protocols in Molecular Biology, Chapter 11 (Ausubel et al., eds., John Wiley and Sons, New York, 5th ed. 2002).

The terms “cereblon” or “CRBN” and similar terms refers to the polypeptides (“polypeptides,” “peptides,” and “proteins” are used interchangeably herein) comprising the amino acid sequence of any CRBN, such as a human CRBN protein (e.g., human CRBN isoform 1, GenBank Accession No. NP_057386; or human CRBN isoforms 2, GenBank Accession No. NP_001166953, each of which is herein incorporated by reference in its entirety), and related polypeptides, including SNP variants thereof. Related CRBN polypeptides include allelic variants (e.g., SNP variants), splice variants, fragments, derivatives, substitution variant, deletion variant, insertion variant, fusion polypeptides, and interspecies homologs, which, in certain embodiments, retain CRBN activity and/or are sufficient to generate an anti-CRBN immune response.

The term “variable region” or “variable domain” refers to a portion of a light or heavy chain of an antibody, typically ranging from about 120 to about 130 amino acids at the amino terminal of the heavy chain and from about 100 to about 110 amino acids at the amino terminal of the light chain, which differs extensively in sequence among antibodies and confers the binding specificity of each antibody to its particular antigen. The variability in sequence is concentrated in those regions called complementarity determining regions (CDRs), while the more conserved regions in the variable domain are called framework regions (FR). The CDRs of the light and heavy chains are primarily responsible for the interaction of the antibody with antigen. Numbering of amino acid positions used herein is according to the Kabat numbering, as in Kabat et al., Sequences of Proteins of Immunological Interest, U.S. Department of Health and Human Services, NIH Publication No. 91-3242 (5th ed. 1991). In some embodiments, the variable region is a human variable region.

The term “expressed” or “expression” as used herein refers to the transcription from a gene to give an RNA nucleic acid molecule at least complementary in part to a region of one of the two nucleic acid strands of the gene. The term “expressed” or “expression” as used herein also refers to the translation from the RNA molecule to give a protein, a polypeptide, or a portion thereof.

The term “level” refers to the amount, accumulation, or rate of a biomarker molecule. A level can be represented, for example, by the amount or the rate of synthesis of a messenger RNA (mRNA) encoded by a gene, the amount or the rate of synthesis of a polypeptide or protein encoded by a gene, or the amount or the rate of synthesis of a biological molecule accumulated in a cell or biological fluid. The term “level” refers to an absolute amount of a molecule in a sample or a relative amount of the molecule, determined under steady-state or non-steady-state conditions.

An mRNA that is “upregulated” is generally increased upon a given treatment or condition. An mRNA that is “downregulated” generally refers to a decrease in the level of expression of the mRNA in response to a given treatment or condition. In some situations, the mRNA level can remain unchanged upon a given treatment or condition. An mRNA from a patient sample can be “upregulated” when treated with a drug, as compared to a non-treated control. This upregulation can be, for example, an increase of about 5%, about 10%, about 20%, about 30%, about 40%, about 50%, about 60%, about 70%, about 80%, about 90%, about 100%, about 200%, about 300%, about 500%, about 1,000%, about 5,000%, or more of the comparative control mRNA level. Alternatively, an mRNA can be “downregulated”, or expressed at a lower level, in response to administration of certain compounds or other agents. A downregulated mRNA can be, for example, present at a level of about 99%, about 95%, about 90%, about 80%, about 70%, about 60%, about 50%, about 40%, about 30%, about 20%, about 10%, about 1%, or less of the comparative control mRNA level.

Similarly, the level of a polypeptide or protein biomarker from a patient sample can be increased when treated with a drug, as compared to a non-treated control. This increase can be about 5%, about 10%, about 20%, about 30%, about 40%, about 50%, about 60%, about 70%, about 80%, about 90%, about 100%, about 200%, about 300%, about 500%, about 1,000%, about 5,000%, or more of the comparative control protein level. Alternatively, the level of a protein biomarker can be decreased in response to administration of certain compounds or other agents. This decrease can be, for example, present at a level of about 99%, about 95%, about 90%, about 80%, about 70%, about 60%, about 50%, about 40%, about 30%, about 20%, about 10%, about 1%, or less of the comparative control protein level.

The terms “determining,” “measuring,” “evaluating,” “assessing,” and “assaying” as used herein generally refer to any form of measurement, and include determining whether an element is present or not. These terms include quantitative and/or qualitative determinations. Assessing may be relative or absolute. “Assessing the presence of” can include determining the amount of something present, as well as determining whether it is present or absent.

The terms “nucleic acid” and “polynucleotide” are used interchangeably herein to describe a polymer of any length composed of nucleotides, e.g., deoxyribonucleotides or ribonucleotides, or compounds produced synthetically, which can hybridize with naturally occurring nucleic acids in a sequence specific manner analogous to that of two naturally occurring nucleic acids, e.g., can participate in Watson-Crick base pairing interactions. As used herein in the context of a polynucleotide sequence, the term “bases” (or “base”) is synonymous with “nucleotides” (or “nucleotide”), i.e., the monomer subunit of a polynucleotide. The terms “nucleoside” and “nucleotide” are intended to include those moieties that contain not only the known purine and pyrimidine bases, but also other heterocyclic bases that have been modified. Such modifications include methylated purines or pyrimidines, acylated purines or pyrimidines, alkylated riboses or other heterocycles. In addition, the terms “nucleoside” and “nucleotide” include those moieties that contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like. “Analogues” refer to molecules having structural features that are recognized in the literature as being mimetics, derivatives, having analogous structures, or other like terms, and include, for example, polynucleotides incorporating non-natural nucleotides, nucleotide mimetics such as 2′-modified nucleosides, peptide nucleic acids, oligomeric nucleoside phosphonates, and any polynucleotide that has added substituent groups, such as protecting groups or linking moieties.

The term “complementary” refers to specific binding between polynucleotides based on the sequences of the polynucleotides. As used herein, a first polynucleotide and a second polynucleotide are complementary if they bind to each other in a hybridization assay under stringent conditions, e.g., if they produce a given or detectable level of signal in a hybridization assay. Portions of polynucleotides are complementary to each other if they follow conventional base-pairing rules, e.g., A pairs with T (or U) and G pairs with C, although small regions (e.g., fewer than about 3 bases) of mismatch, insertion, or deleted sequence may be present.

“Sequence identity” or “identity” in the context of two nucleic acid sequences refers to the residues in the two sequences that are the same when aligned for maximum correspondence over a specified comparison window, and can take into consideration of additions, deletions, and substitutions.

The term “substantial identity” or “homologous” in their various grammatical forms in the context of polynucleotides generally means that a polynucleotide comprises a sequence that has a desired identity, for example, at least 60% identity, preferably at least 70% identity, more preferably at least 80% identity, still more preferably at least 90% identity, and even more preferably at least 95% identity, compared to a reference sequence. Another indication that nucleotide sequences are substantially identical is if two molecules hybridize to each other under stringent conditions.

The terms “isolated” and “purified” refer to isolation of a substance (such as mRNA, DNA, or protein) such that the substance comprises a substantial portion of the sample in which it resides, i.e., greater than the portion of the substance that is typically found in its natural or un-isolated state. Typically, a substantial portion of the sample comprises, e.g., greater than 1%, greater than 2%, greater than 5%, greater than 10%, greater than 20%, greater than 50%, or more, usually up to about 90%-100% of the sample. For example, a sample of isolated mRNA can typically comprise at least about 1% total mRNA. Techniques for purifying polynucleotides are well known in the art and include, for example, gel electrophoresis, ion-exchange chromatography, affinity chromatography, flow sorting, and sedimentation according to density.

As used herein, the term “bound” indicates direct or indirect attachment. In the context of chemical structures, “bound” (or “bonded”) may refer to the existence of a chemical bond directly joining two moieties or indirectly joining two moieties (e.g., via a linking group or any other intervening portion of the molecule). The chemical bond may be a covalent bond, an ionic bond, a coordination complex, hydrogen bonding, van der Waals interactions, or hydrophobic stacking, or may exhibit characteristics of multiple types of chemical bonds. In certain instances, “bound” includes embodiments where the attachment is direct and embodiments where the attachment is indirect.

The term “sample” as used herein relates to a material or mixture of materials, typically, although not necessarily, in fluid form, containing one or more components of interest.

“Biological sample” as used herein refers to a sample obtained from a biological subject, including a sample of biological tissue or fluid origin, obtained, reached, or collected in vivo or in situ. A biological sample also includes samples from a region of a biological subject containing precancerous or cancer cells or tissues. Such samples can be, but are not limited to, organs, tissues, and cells isolated from a mammal. Exemplary biological samples include but are not limited to cell lysate, a cell culture, a cell line, a tissue, oral tissue, gastrointestinal tissue, an organ, an organelle, a biological fluid, a blood sample, a urine sample, a skin sample, and the like. Preferred biological samples include, but are not limited to, whole blood, partially purified blood, PBMC, tissue biopsies, and the like.

The term “analyte” as used herein refers to a known or unknown component of a sample.

The term “capture agent” as used herein refers to an agent that binds an mRNA or protein through an interaction that is sufficient to permit the agent to bind and to concentrate the mRNA or protein from a heterogeneous mixture.

The term “probe” as used herein refers to a capture agent that is directed to a specific target mRNA biomarker sequence. Accordingly, each probe of a probe set has a respective target mRNA biomarker. A probe/target mRNA duplex is a structure formed by hybridizing a probe to its target mRNA biomarker.

The term “nucleic acid probe” or “oligonucleotide probe” refers to a nucleic acid capable of binding to a target nucleic acid of complementary sequence, such as the mRNA biomarkers provided herein, usually through complementary base pairing by forming hydrogen bond. As used herein, a probe may include natural (e.g., A, G, C, or T) or modified bases (7-deazaguanosine, inosine, etc.). In addition, the bases in a probe may be joined by a linkage other than a phosphodiester bond, so long as it does not interfere with hybridization. It will be understood by one of skill in the art that probes may bind target sequences lacking complete complementarity with the probe sequence depending upon the stringency of the hybridization conditions. The probes are preferably directly labeled with tags, for example, chromophores, lumiphores, chromogens, or indirectly labeled with biotin to which a streptavidin complex may later bind. By assaying for the presence or absence of the probe, one can detect the presence or absence of a target mRNA biomarker of interest.

The term “stringent assay conditions” refers to conditions that are compatible to produce binding pairs of nucleic acids, e.g., probes and target mRNAs, of sufficient complementarity to provide for the desired level of specificity in the assay while being generally incompatible to the formation of binding pairs between binding members of insufficient complementarity to provide for the desired specificity. The term “stringent assay conditions” generally refers to the combination of hybridization and wash conditions.

A “label” or “detectable moiety” in reference to a nucleic acid refers to a composition that, when linked with a nucleic acid, renders the nucleic acid detectable, for example, by spectroscopic, photochemical, biochemical, immunochemical, or chemical means. Exemplary labels include, but are not limited to, radioactive isotopes, magnetic beads, metallic beads, colloidal particles, fluorescent dyes, enzymes, biotin, digoxigenin, haptens, and the like. A “labeled nucleic acid or oligonucleotide probe” is generally one that is bound, either covalently through a linker or a chemical bond, or noncovalently through ionic bonds, van der Waals forces, electrostatic attractions, hydrophobic interactions, or hydrogen bonds, to a label such that the presence of the nucleic acid or probe can be detected by detecting the presence of the label bound to the nucleic acid or probe.

The term “polymerase chain reaction” or “PCR” as used herein generally refers to a procedure wherein small amounts of a nucleic acid, RNA and/or DNA, are amplified as described, for example, in U.S. Pat. No. 4,683,195. Generally, sequence information from the ends or beyond of the region of interest needs to be available, such that oligonucleotide primers can be designed; these primers will be identical or similar in sequence to opposite strands of the template to be amplified. The 5′ terminal nucleotides of the two primers may coincide with the ends of the amplified material. PCR can be used to amplify specific RNA sequences, specific DNA sequences from total genomic DNA, and cDNA transcribed from total cellular RNA, bacteriophage, or plasmid sequences, etc. See generally Mullis et al., Cold Spring Harbor Symp. Quant. Biol. 1987, 51:263-273; PCR Technology (Stockton Press, NY, Erlich, ed., 1989).

The term “cycle number” or “C_{T}” when used herein in reference to PCR methods, refers to the PCR cycle number at which the fluorescence level passes a given set threshold level. The C_{T} measurement can be used, for example, to approximate levels of mRNA in an original sample. The C_{T} measurement is often used in terms of “dC_{T}” or the “difference in the C_{T}” score, when the C_{T} of one nucleic acid is subtracted from the C_{T} of another nucleic acid.

As used herein and unless otherwise indicated, the term “pharmaceutically acceptable salt” encompasses non-toxic acid and base addition salts of the compound to which the term refers. Acceptable non-toxic acid addition salts include those derived from organic and inorganic acids know in the art, which include, for example, hydrochloric acid, hydrobromic acid, phosphoric acid, sulfuric acid, methanesulphonic acid, acetic acid, tartaric acid, lactic acid, succinic acid, citric acid, malic acid, maleic acid, sorbic acid, aconitic acid, salicylic acid, phthalic acid, embolic acid, enanthic acid, and the like. Compounds that are acidic in nature are capable of forming salts with various pharmaceutically acceptable bases. The bases that can be used to prepare pharmaceutically acceptable base addition salts of such acidic compounds are those that form non-toxic base addition salts, i.e., salts containing pharmacologically acceptable cations such as, but not limited to, alkali metal or alkaline earth metal salts (calcium, magnesium, sodium, or potassium salts in particular). Suitable organic bases include, but are not limited to, N,N-dibenzylethylenediamine, chloroprocaine, choline, diethanolamine, ethylenediamine, meglumaine (N-methylglucamine), lysine, and procaine.

As used herein and unless otherwise indicated, the term “solvate” means a compound provided herein or a salt thereof that further includes a stoichiometric or non-stoichiometric amount of solvent bound by non-covalent intermolecular forces. Where the solvent is water, the solvate is a hydrate.

As used herein and unless otherwise indicated, the term “co-crystal” means a crystalline form that contains more than one compound in a crystal lattice. Co-crystals include crystalline molecular complexes of two or more non-volatile compounds bound together in a crystal lattice through non-ionic interactions. As used herein, co-crystals include pharmaceutical co-crystals wherein the crystalline molecular complexes containing a therapeutic compound and one or more additional non-volatile compound(s) (referred to herein as counter-molecule(s)). A counter-molecule in a pharmaceutical co-crystal is typically a non-toxic pharmaceutically acceptable molecule, such as, for example, food additives, preservatives, pharmaceutical excipients, or other active pharmaceutical ingredients (API). In some embodiments, pharmaceutical co-crystals enhance certain physicochemical properties of drug products (e.g., solubility, dissolution rate, bioavailability, and/or stability) without compromising the chemical structural integrity of the API. See, e.g., Jones et al., MRS Bulletin 2006, 31,875-879; Trask, Mol. Pharmaceutics 2007, 4(3):301-309; Schultheiss & Newman, Crystal Growth & Design 2009, 9(6):2950-2967; Shan & Zaworotko, Drug Discovery Today 2008, 13(9/10):440-446; and Vishweshwar et al., J. Pharm. Sci. 2006, 95(3):499-516.

As used herein, and unless otherwise specified, the term “stereoisomer” encompasses all enantiomerically/stereomerically pure and enantiomerically/stereomerically enriched compounds of this invention.

As used herein and unless otherwise indicated, the term “stereomerically pure” means a composition that comprises one stereoisomer of a compound and is substantially free of other stereoisomers of that compound. For example, a stereomerically pure composition of a compound having one chiral center will be substantially free of the opposite enantiomer of the compound. A stereomerically pure composition of a compound having two chiral centers will be substantially free of other diastereomers of the compound. A typical stereomerically pure compound comprises greater than about 80% by weight of one stereoisomer of the compound and less than about 20% by weight of other stereoisomers of the compound, more preferably greater than about 90% by weight of one stereoisomer of the compound and less than about 10% by weight of the other stereoisomers of the compound, even more preferably greater than about 95% by weight of one stereoisomer of the compound and less than about 5% by weight of the other stereoisomers of the compound, and most preferably greater than about 97% by weight of one stereoisomer of the compound and less than about 3% by weight of the other stereoisomers of the compound.

As used herein and unless otherwise indicated, the term “stereomerically enriched” means a composition that comprises greater than about 60% by weight of one stereoisomer of a compound, preferably greater than about 70% by weight, more preferably greater than about 80% by weight of one stereoisomer of a compound. As used herein and unless otherwise indicated, the term “enantiomerically pure” means a stereomerically pure composition of a compound having one chiral center. Similarly, the term “stereomerically enriched” means a stereomerically enriched composition of a compound having one chiral center.

As used herein, and unless otherwise specified, the term “prodrug” means a derivative of a compound that can hydrolyze, oxidize, or otherwise react under biological conditions (in vitro or in vivo) to provide the compound. Examples of prodrugs include, but are not limited to, compounds that comprise biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Other examples of prodrugs include compounds that comprise —NO, —NO₂, —ONO, or —ONO₂ moieties. Prodrugs can typically be prepared using well-known methods, such as those described in Burger's Medicinal Chemistry and Drug Discovery, 172-178, 949-982 (Manfred E. Wolff, ed., 5th ed. 1995), and Design of Prodrugs (H. Bundgaard, ed., Elselvier, N.Y. 1985).

As used herein, and unless otherwise specified, the terms “biohydrolyzable carbamate,” “biohydrolyzable carbonate,” “biohydrolyzable ureide” and “biohydrolyzable phosphate” mean a carbamate, carbonate, ureide, and phosphate, respectively, of a compound that either: 1) does not interfere with the biological activity of the compound but can confer upon that compound advantageous properties in vivo, such as uptake, duration of action, or onset of action; or 2) is biologically inactive but is converted in vivo to the biologically active compound. Examples of biohydrolyzable carbamates include, but are not limited to, lower alkylamines, substituted ethylenediamines, amino acids, hydroxyalkylamines, heterocyclic and heteroaromatic amines, and polyether amines.

It should also be noted compounds can contain unnatural proportions of atomic isotopes at one or more of the atoms. For example, the compounds may be radiolabeled with radioactive isotopes, such as for example tritium (³H), iodine-125 (¹²⁵I), sulfur-35 (³⁵S), or carbon-14 (¹⁴C), or may be isotopically enriched, such as with deuterium (²H), carbon-13 (¹³C), or nitrogen-15 (¹⁵N). As used herein, an “isotopologue” is an isotopically enriched compound. The term “isotopically enriched” refers to an atom having an isotopic composition other than the natural isotopic composition of that atom. “Isotopically enriched” may also refer to a compound containing at least one atom having an isotopic composition other than the natural isotopic composition of that atom. The term “isotopic composition” refers to the amount of each isotope present for a given atom. Radiolabeled and isotopically encriched compounds are useful as therapeutic agents, e.g., cancer and inflammation therapeutic agents, research reagents, e.g., binding assay reagents, and diagnostic agents, e.g., in vivo imaging agents. All isotopic variations of the compounds as described herein, whether radioactive or not, are intended to be encompassed within the scope of the embodiments provided herein. In some embodiments, there are provided isotopologues of the compounds, for example, the isotopologues are deuterium, carbon-13, or nitrogen-15 enriched compounds. In some embodiments, isotopologues provided herein are deuterium enriched compounds. In some embodiments, isotopologues provided herein are deuterium enriched compounds, where the deuteration occurs on the chiral center. In some embodiments, provided herein are isotopologues of the compounds of Formula I, where deuteration occurs on the chiral center. In some embodiments, provided herein are isotopologues of Compound C, where deuteration occurs on the chiral center.

As used herein, and unless otherwise indicated, the term “alkyl” refers to a saturated straight chain or branched hydrocarbon having number of carbon atoms as specified herein. Representative saturated straight chain alkyls include -methyl, -ethyl, -n-propyl, -n-butyl, -n-pentyl, and -n-hexyl; while saturated branched alkyls include -isopropyl, -sec-butyl, -isobutyl, -tert-butyl, -isopentyl, 2-methylbutyl, 3-methylbutyl, 2-methylpentyl, 3-methylpentyl, 4-methylpentyl, 2-methylhexyl, 3-methylhexyl, 4-methylhexyl, 5-methylhexyl, 2,3-dimethylbutyl, and the like.

As used herein, and unless otherwise specified, the term “cycloalkyl” means a saturated, or partially saturated cyclic alkyl containing from 3 to 15 carbon atoms, without alternating or resonating double bonds between carbon atoms. It may contain from 1 to 4 rings. Examples of unsubstituted cycloalkyls include, but are not limited to, cyclopropyl, cyclobutyl, cyclopentyl, cyclohexyl, and adamantyl. A cycloalkyl may be substituted with one or more of the substituents as defined below.

As used herein, and unless otherwise specified, the term “alkoxy” refers to —O-(alkyl), wherein alkyl is defined herein. Examples of alkoxy include, but are not limited to, —OCH₃, —OCH₂CH₃, —O(CH₂)₂CH₃, —O(CH₂)₃CH₃, —O(CH₂)₄CH₃, and —O(CH₂)₅CH₃.

As used herein, the term “aryl” means a carbocyclic aromatic ring containing from 5 to 14 ring atoms. The ring atoms of a carbocyclic aryl group are all carbon atoms. Aryl ring structures include compounds having one or more ring structures such as mono-, bi-, or tricyclic compounds as well as benzo-fused carbocyclic moieties such as 5,6,7,8-tetrahydronaphthyl, and the like. Representative aryl groups include phenyl, anthracenyl, fluorenyl, indenyl, azulenyl, phenanthrenyl, and naphthyl.

As used herein, and unless otherwise specified, the term “heteroaryl” means an aromatic ring containing from 5 to 14 ring atoms, of which at least one (e.g., one, two, or three) is a heteroatom (e.g., nitrogen, oxygen, or sulfur). Heteroaryl ring structures include compounds having one or more ring structures such as mono-, bi-, or tricyclic compounds, as well as fused heterocyclic moieties. Examples of heteroaryls include, but are not limited to, triazolyl, tetrazolyl, oxadiazolyl, pyridyl, furyl, benzofuranyl, thiophenyl, thiazolyl, benzothiophenyl, benzoisoxazolyl, benzoisothiazolyl, quinolinyl, isoquinolinyl, pyrrolyl, indolyl, oxazolyl, benzoxazolyl, imidazolyl, benzimidazolyl, thiazolyl, benzothiazolyl, isoxazolyl, pyrazolyl, isothiazolyl, pyridazinyl, pyrimidinyl, pyrazinyl, triazinyl, cinnolinyl, phthalazinyl, quinazolinyl, benzoquinazolinyl, quinoxalinyl, acridinyl, pyrimidyl, oxazolyl, benzo[1,3]dioxole, and 2,3-dihydro-benzo[1,4]dioxine.

As used herein, and unless otherwise indicated, the term “heterocycle” means a monocyclic or polycyclic ring comprising carbon and hydrogen atoms, optionally having 1 or 2 multiple bonds, and the ring atoms contain at least one heteroatom, specifically 1 to 3 heteroatoms, independently selected from nitrogen, oxygen, and sulfur. Heterocycle ring structures include, but are not limited to, mono-, bi-, and tri-cyclic compounds. Specific heterocycles are monocyclic or bicyclic. Representative heterocycles include morpholinyl, pyrrolidinonyl, pyrrolidinyl, piperidinyl, piperazinyl, hydantoinyl, valerolactamyl, oxiranyl, oxetanyl, tetrahydrofuranyl, tetrahydropyranyl, tetrahydropyridinyl, tetrahydroprimidinyl, tetrahydrothiophenyl, and tetrahydrothiopyranyl. A heterocyclic ring may be unsubstituted or substituted.

As used herein, and unless otherwise specified, the term “heterocycloalkyl” refers to a cycloalkyl group in which at least one of the carbon atoms in the ring is replaced by a heteroatom (e.g., nitrogen, oxygen, or sulfur).

As used herein, and unless otherwise indicated, the term “alkylenedioxy” refers to multiples of the —CH₂ group with an oxygen atom at each end, the —CH₂ groups optionally substituted with alkyl groups. Examples include —O—CH₂—O-(methylenedioxy), —O—CH₂CH₂—O-(ethylenedioxy), —O—CH₂CH₂CH₂—O-(trimethylenedioxy), —O—CH₂CH₂CH₂CH₂—O-(tetramethylenedioxy), —O—CH(CH₃)CH₂—O-(α-methylethylenedioxy), —O—CH(C₂H₅)CH₂—O-(α-ethylethylenedioxy), etc.

As used herein, and unless otherwise indicated, the term “alkylthio” refers to groups having the formula Y—S—, wherein Y is alkyl as defined above.

The term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 50%, 20%, 15%, 10%, 9%, 8%, 7%, 6%, 5%, 4%, 3%, 2%, 1%, 0.5%, or 0.05% of a given value or range.

It should be noted that if there is a discrepancy between a depicted structure and a name given to that structure, the depicted structure is to be accorded more weight. In addition, if the stereochemistry of a structure or a portion of a structure is not indicated with, for example, bold or dashed lines, the structure or portion of the structure is to be interpreted as encompassing all stereoisomers of it.

The practice of the embodiments provided herein will employ, unless otherwise indicated, conventional techniques of molecular biology, microbiology, and immunology, which are within the skill of those working in the art. Such techniques are explained fully in the literature. Examples of particularly suitable texts for consultation include the following: Sambrook et al., Molecular Cloning: A Laboratory Manual (2d ed. 1989); Glover, ed., DNA Cloning, Volumes I and II (1985); Gait, ed., Oligonucleotide Synthesis (1984); Hames & Higgins, eds., Nucleic Acid Hybridization (1984); Hames & Higgins, eds., Transcription and Translation (1984); Freshney, ed., Animal Cell Culture: Immobilized Cells and Enzymes (IRL Press, 1986); Immunochemical Methods in Cell and Molecular Biology (Academic Press, London); Scopes, Protein Purification: Principles and Practice (Springer Verlag, N.Y., 2d ed. 1987); and Weir & Blackwell, eds., Handbook of Experimental Immunology, Volumes I-IV (1986).

### 5.2 Biomarkers and Methods of Use Thereof

The methods provided herein are based, in part, on the finding that detectable increase or decrease in certain biomarkers are observed in subjects with cancers (e.g., lymphoma, MM, or leukemia), who are responsive to a given treatment (e.g., a compound, such as a compound of Formula I, or a pharmaceutically acceptable salt, solvate, stereoisomer, isotopologue, prodrug, hydrate, co-crystal, clathrate, or a polymorph thereof), and that the levels of these biomarkers may be used for predicting the responsiveness of the subjects to the treatment. In certain embodiments, the compound of Formula I is Compound C.

A “biological marker” or “biomarker” is a substance, the change and/or the detection of which indicates a particular biological state. In some embodiments, the indication is the responsiveness of a disease, e.g., cancer (e.g., lymphoma, MM, or leukemia), to a given treatment (e.g., a compound, such as a compound of Formula I, or a pharmaceutically acceptable salt, solvate, stereoisomer, isotopologue, prodrug, hydrate, co-crystal, clathrate, or a polymorph thereof). In certain embodiments, the compound of Formula I is Compound C.

As described in the Examples and shown in the figures, the levels of certain proteins and/or mRNAs change in response to Compound C treatment. These biomarkers include eRF3a, eRF3b, eRF3c, IKZF1, IKZF3, CK1a, eRF1, BIP, PERK, eIF2a, ATF4, ATF3, DDIT3, PPP1R15A, TNFRSF10B, GADD45A, TNFRSF1A, TNFRSF1B, FAS, FADD, IRE1, XBP1, SEC24D, DNAJB9, EDEM1, EDEM2, HYOU1, ATF6, HSPA5, Caspase 3, Caspase 7, Caspase 8, BID, Caspase 9, PARP, Mcl-1, and BAD. Thus, in some embodiments, the biomarker provided herein is selected from the group consisting of eRF3a, eRF3b, eRF3c, IKZF1, IKZF3, CK1a, PABP1, eRF1, BIP, eEF1α, PERK, eIF2a, ATF4, ATF3, DDIT3, PPP1R15A, TNFRSF10B, GADD45A, TNFRSF1A, TNFRSF1B, FAS, FADD, IRE1, XBP1, SEC24D, DNAJB9, EDEM1, EDEM2, HYOU1, ATF6, HSPA5, Caspase 8, BID, Caspase 9, Caspase 7, Caspase 3, PARP, Mcl-1, and BAD. Each of the biomarkers provided herein includes various isoforms, phosphorylated forms, cleaved forms, modified forms, and splicing variants thereof. For example, PERK includes the phosphorylated form of PERK. eIF2a includes the phosphorylated form of eIF2a. IRE1 includes the phosphorylated form of IRE1. BAD includes the phosphorylated form of BAD (e.g., pS112-BAD). BIP includes the modified form (e.g., C-terminal modified BIP). ATF3 includes the splicing variant of ATF3. Caspase 3 includes the cleaved form of Caspase 3. Caspase 7 includes the cleaved form of Caspase 7. Caspase 8 includes the cleaved form of Caspase 8. Caspase 9 includes the cleaved form of Caspase 9. PARP includes the cleaved form of PARP.

Eukaryotic peptide chain release factor GTP-binding subunit eRF3a is also called GSPT1 (G1 to S phase transition protein 1 homolog). It is involved in translation termination in response to the termination codons UAA, UAG, and UGA, and is also involved in regulation of mammalian cell growth. eRF3a stimulates the activity of eRF1 and is a component of the transient SURF complex, which recruits UPF1 to stalled ribosomes in the context of nonsense-mediated decay (NMD) of mRNAs containing premature stop codons.

Eukaryotic peptide chain release factor GTP-binding subunit eRF3b is also called GSPT2 (G1 to S phase transition protein 2 homolog). Like eRF3a, eRF3b is also involved in translation termination in response to the termination codons UAA, UAG, and UGA, and is a component of the transient SURF complex, which recruits UPF1 to stalled ribosomes in the context of nonsense-mediated decay (NMD) of mRNAs containing premature stop codons. It is suggested that eRF3b plays a role as a potent stimulator of the release factor activity of ETF1, and that it may play a role in cell cycle progression. In addition, eRF3b has been shown to exhibit GTPase activity, which is ribosome- and ETF1-dependent.

HBS1-like protein or HBS1L (also called eRF3c) is a member of the GTP-binding elongation factor family. It is expressed in multiple tissues with the highest expression in heart and skeletal muscle. The intergenic region of this gene and the MYB gene has been identified to be a quantitative trait locus (QTL) controlling fetal hemoglobin level, and this region influences erythrocyte, platelet, and monocyte counts as well as erythrocyte volume and hemoglobin content. DNA polymorphisms at this region associate with fetal hemoglobin levels and pain crises in sickle cell disease.

Activating Transcription Factor 4 (ATF4) is a transcription factor also known as the cAMP-response element binding protein 2 (CREB-2). It belongs to a family of DNA-binding proteins that includes the AP-1 family, CREBs, and CREB-like proteins.

Activating Transcription Factor 3 (ATF3) belongs to the mammalian activation transcription factor/cAMP responsive element-binding (CREB) protein family of transcription factors. The ATF3 gene is induced by a variety of signals, including many of those encountered by cancer cells, and is involved in the complex process of cellular stress response.

DNA-Damage-Inducible Transcript 3 (DDIT3) is a member of the CCAAT/enhancer-binding protein (C/EBP) family of transcription factors. DDIT3 is also known as C/EBP homologous protein (CHOP). The protein functions as a dominant-negative inhibitor by forming heterodimers with other C/EBP members, such as C/EBP and LAP (liver activator protein), and preventing their DNA binding activity. The protein is also implicated in adipogenesis and erythropoiesis, is activated by endoplasmic reticulum stress, and promotes apoptosis. DDIT3 is a multifunctional transcription factor in endoplasmic reticulum (ER) stress response. It plays an essential role in the response to a wide variety of cell stresses and induces cell cycle arrest and apoptosis in response to ER stress.

Casein kinase 1 alpha (CK1a) is the alpha isoform of a monomeric serine-threonine protein kinase. CK1 is involved in a number of cellular processes including DNA repair, cell division, nuclear localization, and membrane transport.

Poly(A) Binding Protein 1 (PABP1) binds to the 3′-poly(A) tail of eukaryotic messenger RNAs via RNA-recognition motifs and shuttles between the nucleus and cytoplasm. The binding of PABP1 to poly(A) promotes ribosome recruitment and translation initiation. PABP1 is part of a small gene family including three protein-coding genes and several pseudogenes.

Eukaryotic Elongation Factor 1 alpha (eEF1α) is the alpha subunit of the elongation factor-1 complex, which is responsible for the enzymatic delivery of aminoacyl tRNAs to the ribosome during protein synthesis. Mammalian eEF1α has two isoforms with high amino acid sequence homology, eEF1α1 and eEF1α2. eEF1α also play a role in the nuclear export of proteins. Upregulation of eEF1α has been reported in certain cancer, such as breast cancer.

PKR-like ER kinase (PERK, also known as EIF2AK3) is an EIF2 alpha kinase that inhibits protein translation. PERK is a endoplasmic reticulum (ER) membrane protein which is involved in both the integrated stress response (ISR) and unfolded protein response (UPR). PERK phosphorylates EIF2a, leading to its inactivation, and a reduction of translational initiation and repression of protein synthesis.

IKAROS Family Zinc Finger 1 (IKZF1, also known as Ikaros) is a transcription factor that belongs to the family of zinc-finger DNA-binding proteins associated with chromatin remodeling. The expression of IKZF1 is restricted to the fetal and adult hemo-lymphopoietic system, and it functions as a regulator of lymphocyte differentiation. Most isoforms share a common C-terminal domain, which contains two zinc finger motifs that are required for hetero- or homo-dimerization, and for interactions with other proteins. The isoforms, however, differ in the number of N-terminal zinc finger motifs that bind DNA and in nuclear localization signal presence, resulting in members with and without DNA-binding properties. Only a few isoforms contain the requisite three or more N-terminal zinc motifs that confer high affinity binding to a specific core DNA sequence element in the promoters of target genes. The non-DNA-binding isoforms are largely found in the cytoplasm, and are thought to function as dominant-negative factors. Overexpression of some dominant-negative isoforms have been associated with B-cell malignancies, such as acute lymphoblastic leukemia (ALL).

IKAROS Family Zinc Finger 3 (IKZF3, also known as Aiolos) is also a member of the Ikaros family of zinc-finger proteins. Three members of this protein family (Ikaros, Aiolos, and Helios) are hematopoietic-specific transcription factors involved in the regulation of lymphocyte development. IKZF3 is a transcription factor that is important in the regulation of B lymphocyte proliferation and differentiation. Both IKZF1 and IKZF3 can participate in chromatin remodeling. Regulation of gene expression in B lymphocytes by IKZF3 is complex as it appears to require the sequential formation of IKZF1 homodimers, IKZF1/IKZF3 heterodimers, and IKZF3 homodimers.

Eukaryotic Release Factor 1 (eRF1) is a protein that recognizes all three stop codons in the mRNA sequence and terminates protein translation by releasing the nascent polypeptide. It is a component of the SURF complex that promotes degradation of prematurely terminated mRNAs via the mechanism of nonsense-mediated mRNA decay (NMD).

SEC24D is a member of the SEC24 subfamily of the SEC23/SEC24 family, which is involved in vesicle trafficking. SEC24D is implicated in the shaping of the vesicle, cargo selection and concentration.

DNAJB9 is a member of the J protein family. J proteins regulate the ATPase activity of hsp70s. DNAJB9 is induced during UPR by the ER stress and plays a role in protecting stressed cells from apoptosis.

DNAJC6 is a also member of the J protein family, which regulates molecular chaperone activity by stimulating ATPase activity. DNAJ proteins may have up to 3 distinct domains: a conserved 70-amino acid J domain, usually at the N terminus, a glycine/phenylalanine (G/F)-rich region, and a cysteine-rich domain containing 4 motifs resembling a zinc finger domain.

X-Box Binding Protein 1 (XBP1) is a transcription factor that regulates MHC class II genes by binding to a promoter element referred to as an X box. It is a bZIP protein, identified as a cellular transcription factor that binds to an enhancer in the promoter of the T cell leukemia virus type 1 promoter. It may increase expression of viral proteins by acting as the DNA binding partner of a viral transactivator. XBP1 functions as a transcription factor regulating UPR during the ER stress.

ER Degradation Enhancer Mannosidase Alpha-Like 1 (EDEM1) and ER Degradation Enhancer, Mannosidase Alpha-Like 2 (EDEM2) are directly involved in ER-associated degradation (ERAD) and targets misfolded glycoproteins for degradation in an N-glycan-independent manner.

Hypoxia Up-Regulated 1 (HYOU1) belongs to the heat shock protein 70 family. A cis-acting segment in the 5′-UTR of HYOU1 is involved in stress-dependent induction, resulting in the accumulation of HYOU1 in the ER under hypoxic conditions. HYOU1 plays an important role in protein folding and secretion in the ER. HYOU1 is also up-regulated in tumors, especially in breast tumors, and is associated with tumor invasiveness.

Heat Shock 70 kDa Protein 5 (HSPA5, also known as BIP) is a member of the heat shock protein 70 family. BIP is an ER luminal KDEL protein that requires binding with KDEL receptor in the Cis-Golgi to be retro-transported into the ER lumen for retention. BIP interacts with the ER luminal domain of UPR sensors PERK, IRE1, and ATF6 to prevent their activation. Reduction of BIP C-terminal immunoreactivity indicates a mislocalization of BIP, which presumably leads to its dissociation from PERK, IRE1, and ATF6 and induces UPR.

Eukaryotic Translation Initiation Factor 2a (EIF2a) directs methionyl-tRNAi binding to 40S ribosomal subunits and catalyzes the formation of puromycin-sensitive 80S preinitiation complexes. IL-6 signaling pathway and TGF-β receptor signaling are mmong its related pathways.

Protein Phosphatase 1 Regulatory Subunit 15A (PPP1R15A) belongs to a group of genes, whose mRNA levels are increased following treatment with DNA-damaging agents and stressful growth arrest conditions. In certain cell lines, the induction of PPP1R15A by ionizing radiation occurs regardless of p53 status, and its protein response is correlated with apoptosis following ionizing radiation. GPCR signaling is one of PPP1R15A related pathways.

Growth Arrest and DNA-Damage-Inducible 45 Alpha (GADD45A) is a member of a family of genes, whose mRNA levels are increased following treatment with DNA-damaging agents and stressful growth arrest conditions. GADD45A mediates activation of the p38/JNK pathway via MTK1/MEKK4 kinase, thereby responding to environmental stresses. The DNA damage-induced transcription of this gene is mediated by both p53-dependent and -independent mechanisms.

Tumor Necrosis Factor Receptor Superfamily Member 1A (TNFRSF1A) is a member of the TNF-receptor family. It is one of the major receptors for TNF-alpha. TNFRSF1A activates NF-κB, mediates apoptosis, and regulates inflammation. Antiapoptotic protein BCL2-associated athanogene 4 (BAG4/SODD) and adaptor proteins TRADD and TRAF2 interact with TNFRSF1A, and thus play regulatory roles in the signal transduction mediated by TNFRSF1A. The adapter molecule FADD recruits Caspase-8 to the activated TNFRSF1A. The resulting death-inducing signaling complex (DISC) performs Caspase-8 proteolytic activation, which initiates the subsequent cascade of cysteine-aspartic acid protease (caspase)-mediated apoptosis.

Tumor Necrosis Factor Receptor Superfamily Member 1B (TNFRSF1B) is also a member of the TNF-receptor family. TNFRSF1B associates with TNF-receptor 1, and the heterocomplex recruits two anti-apoptotic proteins, c-IAP1 and c-IAP2, which possess E3 ubiquitin ligase activity. c-IAP1 promotes TNF-induced apoptosis by the ubiquitination and degradation of TNF-receptor-associated factor 2, which mediates anti-apoptotic signals.

Tumor Necrosis Factor Receptor Superfamily Member 10B (TNFRSF10B) is a member of the TNF-receptor family and contains an intracellular death domain. Upon activation by TNF-related apoptosis inducing ligand (TNFSF10/TRAIL/APO-2L), TNFRSF10B transduces an apoptosis signal. FADD, a death domain containing adaptor protein, is required for the apoptosis mediated by TNFRSF10B.

Caspase 8 is a member of the caspase family. Sequential activation of caspases plays a central role in apoptosis. Caspases exist as inactive proenzymes composed of a large protease subunit, a small protease subunit, and a prodomain. Activation of caspases requires proteolysis to generate a heterodimeric enzyme consisting of the large and small subunits. Caspase 8 is involved in the programmed cell death induced by FAS and other apoptotic stimuli. Caspase 8 may interact with Fas-interacting protein FADD through the N-terminal FADD-like death effector domain.

BH3 Interacting Domain (BID) is a death agonist that heterodimerizes with either agonist BAX or antagonist BCL2. BID is a member of the BCL-2 family of cell death regulators. It mediates mitochondrial damage induced by Caspase 8. Caspase 8 cleaves BID, then the C-terminal part of BID translocates to mitochondria and triggers cytochrome c release.

Caspase 9 is a member of the caspase family. Caspase 9 activation is one of the earliest in the caspase activation cascade. Caspase 9 undergoes autoproteolysis and activation by the apoptosome, a protein complex of cytochrome c and the apoptotic peptidase activating factor 1. Caspase 9 is a tumor suppressor and plays a central role in apoptosis.

Caspase 3 is also a member of the caspase family. It cleaves and activates Caspases 6, 7, and 9. Caspase 3 itself is processed by Caspases 8, 9, and 10.

Caspase 7 also belongs to the caspase family. The precursor of Caspase 7 is cleaved by Caspase 3 and 10. It is activated upon cell death stimuli and induces apoptosis.

Poly ADP-Ribose Polymerase (PARP) is a family of proteins involved in regulating various important cellular processes such as differentiation, proliferation, and tumor transformation. PARP also regulates the molecular events involved in cell recovery from DNA damage.

Fas Cell Surface Death Receptor (FAS) is a member of the TNF-receptor family. It contains a death domain. FAS plays a central role in regulting programmed cell death and has been involved in various malignancies and diseases of the immune system. The interaction of FAS with its ligand allows the formation of a death-inducing signaling complex that includes Fas-associated death domain protein (FADD), Caspase 8, and Caspase 10. The autoproteolytic processing of the caspases in the complex triggers a downstream caspase cascade and leads to apoptosis.

Fas-Associated via Death Domain (FADD) interacts with various cell surface receptors and mediates cell apoptotic signals. FADD can be recruited by FAS, TNF receptor, TNFRSF25, and TNFSF10/TRAIL-receptor through its C-terminal death domain, and participates in the death signaling initiated by these receptors. Interaction of FADD with the receptors reveals the N-terminal effector domain of FADD, thus allows it to recruit Caspase-8 and thereby activates the caspase cascade.

Inositol-requiring enzyme 1 (IRE1, also known as ERN1) is a transmembrane ER protein that possesses kinase and endonuclease domains. IRE1 regulates the degradation of misfolded proteins, as part of the UPR pathway. IRE1 catalyzes the splicing of XBP1 mRNA so that the active form of XBP1 protein is produced. Active XBP1, as a transcription factor, upregulates genes involved in the ERAD pathway and induces XBP1 expression and the synthesis of ER chaperones.

Activating Transcription Factor 6 (ATF6) activates target genes for the UPR during ER stress. ATF is a transmembrane ER protein and functions as an ER stress sensor/transducer. Following ER stress-induced proteolysis, ATF functions as a nuclear transcription factor via a ER stress response element (ERSE) present in the promoters of genes encoding ER chaperones.

Myeloid Cell Leukemia 1 (Mcl-1) is a member of the BCL-2 family. BCL-2 family members are regulators of programmed cell death. Alternative splicing results in multiple transcript variants. The longest gene product (isoform 1) inhibits apoptosis and enhances cell survival, while the shorter gene products (isoform 2 and isoform 3) promote apoptosis and induce cell death.

BCL2-Associated Agonist of Cell Death (BAD) is a member of the BCL-2 family. BAD promotes cell apoptosis by forming heterodimers with BCL-xL and BCL-2 and reversing their death repressor activity. Phosphorylation of BAD regulates its proapoptotic activity. Protein kinases AKT and MAP kinase, and protein phosphatase calcineurin are involved in the regulation of BAD.

In certain embodiments of the various methods provided herein, the biomarker is a protein that is directly or indirectly affected by cereblon (CRBN), for example through protein-protein interactions (e.g., certain CRBN substrates or downstream effectors thereof), or through various cellular pathways (e.g., signal transduction pathways). In specific embodiments, the biomarker is a CRBN-associated protein (CAP). In some embodiments, the biomarker is mRNA of a protein that is directly or indirectly affected by CRBN. In other embodiments, the biomarker is cDNA of a protein that is directly or indirectly affected by CRBN. At least two isoforms of the protein CRBN exist, which are 442 and 441 amino acids long, respectively. CRBN has recently been identified as a key molecular target that binds to thalidomide to cause birth defects. See Ito et al., Science 2010, 327:1345-1350. Damaged DNA-binding protein 1 (DDB1) was found to interact with CRBN and, thus, was indirectly associated with thalidomide. Moreover, thalidomide was able to inhibit auto-ubiquitination of CRBN in vitro, suggesting that thalidomide is an E3 ubiquitin-ligase inhibitor. Id. Importantly, this activity was inhibited by thalidomide in wild-type cells, but not in cells with mutated CRBN binding sites that prevent thalidomide binding. Id. The thalidomide binding site was mapped to a highly conserved C-terminal 104 amino acid region in CRBN. Id. Individual point mutants in CRBN, Y384A and W386A, were both defective for thalidomide binding, with the double mutant having the lowest thalidomide-binding activity. Id. A link between CRBN and the teratogenic effect of thalidomide was confirmed in animal models of zebra-fish and chick embryos. Id.

It is yet to be established whether binding of thalidomide or other drugs to CRBN, the CRBN E3 ubiquitin-ligase complex, or one or more substrates of CRBN, is required for the beneficial effects of these drugs. Understanding the interactions between these drugs and CRBN or CRBN-associated proteins will facilitate elucidating molecular mechanisms of drug efficacy and/or toxicity and may lead to development of new drugs with improved efficacy and toxicity profiles.

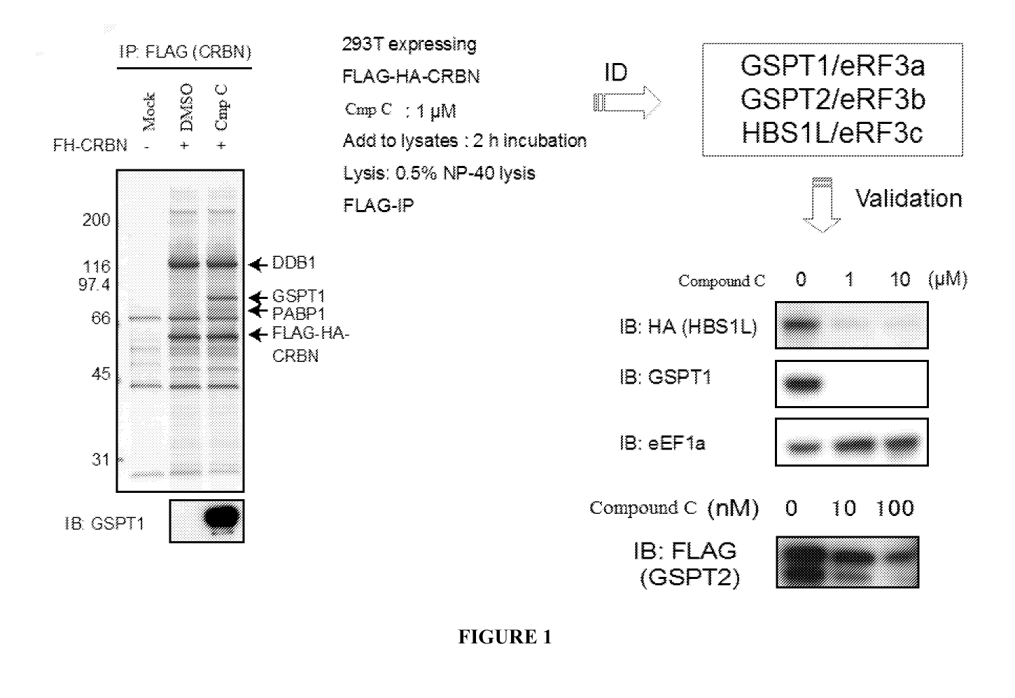

As shown in the Examples and FIGS. 1-3, the levels of certain CAP changes in response to Compound C treatment, such as eRF3a, eRF3b, eRF3c, IKZF1, IKZF3, and CK1a. Thus, in some embodiments, the biomarker is a CAP selected from the group consisting of eRF3a, eRF3b, eRF3c, IKZF1, IKZF3, and CK1a. In some embodiments, the biomarker is an eRF3 family member, such as eRF3a, eRF3b, and eRF3c. In a specific embodiment, the biomarker is eRF3a. In another specific embodiment, the biomarker is eRF3b. In yet another specific embodiment, the biomarker is eRF3c. In yet another specific embodiment, the biomarker is IKZF1. In yet another embodiment, the biomarker is IKZF3. In yet another embodiment, the biomarker is CK1a. In other embodiments, the biomarker is a binding partner of, downstream effector of, or a factor in a cellular pathway impacted by eRF3a, eRF3b, eRF3c, IKZF1, IKZF3, and CK1a. For example, in some embodiments, the biomarker is a binding partner of, downstream effector of, or a factor in a cellular pathway impacted by an eRF3 family member. In a specific embodiment, the biomarker is a binding partner of eRF3a, such as eRF1.

As shown in the Examples, the level of eRF3a, eRF3b, or eRF3c decreases as compared to a reference in response to Compound C treatment. Downregulation of these eRF3 family members result in protein misfolding and/or aggregation, protein mislocation, and direct change of protein function, among other effects. One cellular pathway affected is unfolded protein response (UPR), which is a cellular stress response related to the endoplasmic reticulum (ER). Thus, a factor or a protein involved in UPR or a downstream pathway thereof can be used as a biomarker according to the present disclosure. The pathways related to UPR include, but not limited to, PERK/ATF4/DDIT3 signaling pathway (or PERK related signaling pathway) and related apoptosis pathway, XBP1 signaling pathway (or XBP1 related signaling pathway), and ATF6 signaling pathway (ATF6 related signaling pathway). Thus, in some embodiments, the biomarker provided herein has a function in ER stress pathway. In some embodiments, the biomarker provided herein has a function in UPR pathway. In certain embodiments, the biomarker provided herein has a function in PERK related signaling pathway. In other embodiments, the biomarker provided herein has a function in XBP1 related signaling pathway. In yet other embodiments, the biomarker provided herein has a function in ATF6 related signaling pathway. In some embodiments, the biomarker provided herein has a function in FAS/FADD related signaling and apoptosis pathway.

PERK related signaling pathway is one of the signaling pathways activated upon UPR activation. It attenuates translation and prevents translational overloading of the ER. PERK activates itself by oligomerization and autophosphorylation of its luminal domain. The activated PERK causes translational attenuation by directly phosphorylating eIF2. This also produces translational attenuation of the protein machinery involved in the cell cycle, producing cell cycle arrest in the G1 phase. PERK related signaling pathway includes any downstream pathways that are directly or indirectly affected by PERK pathway. Components involved in PERK related signaling pathway include, but not limited to, PERK, eIF2a, ATF4, ATF3, PPP1R15A, TNFRSF10B, DDIT3, GADD45A, TNFRSF1A, TNFRSF1B, FAS, and FADD.

XBP1 related signaling pathway is another signaling pathway activated during UPR. Upon UPR activation, IRE1, an ER transmembrane receptor, activates itself by homodimerization and transautophosphorylation. The activated IRE1 luminal domain is able to activate the transcription factor XBP1 mRNA by splicing a 252 bp intron. The activated XBP1 upregulates expression of UPR-related genes by directly binding to the stress element promoters of these target genes. Components involved in XBP1 related signaling pathway include, but not limited to, IRE1, XBP1, SEC24D, DNAJB9, DNAJC6, EDEM1, EDEM2, and HYOU1.

ATF6 related signaling pathway is also activated during UPR. Like PERK and IRE1, ATF6 is an ER transmembrane receptor. Upon HSPA5 dissociation from ATF6 during UPR activation, the entire 90 kDa ATF6 translocates to the Golgi, where it is cleaved by proteases to form an active 50 kDa transcription factor that translocates to the nucleus. The 50 kDa ATF6 binds to stress element promoters upstream of genes that are upregulated in the UPR. Components involved in ATF6 related signaling pathway include, but not limited to, ATF6, XBP1, EDEM1, EDEM2, HYOU1, and HSPA5.

FAS/FADD related signaling and apoptosis pathway is a downstream pathway that may be activated upon UPR. When the primary goals of UPR (such as attenuating protein translation, degrading misfolded proteins, and activating signaling pathways that increase production of chaperone proteins) are not achieved, UPR directs towards apoptosis. Upon stimulation by ligand, FAS receptor trimerizes. FADD, an adaptor protein, bridges FAS to procaspases 8 and 10 to form the death-inducing signaling complex (DISC) during apoptosis. Components involved in FAS/FADD related signaling and apoptosis pathway include, but not limited to, FAS, FADD, Caspase 8, BID, Caspase 9, Caspase 3, Caspase 7, and PARP.

For example, as shown in the Examples, the levels of proteins in PERK related signaling pathway change in response to Compound C treatment, such as PERK, EIF2a, ATF4, ATF3, DDIT3, PPP1R15A, TNFRSF10B, GADD45A, TNFRSF1A, TNFRSF1B, FAS, and FADD. Thus, in some embodiments, the biomarker provided herein is selected from the group consisting of PERK, EIF2a, ATF4, ATF3, DDIT3, PPP1R15A, TNFRSF10B, GADD45A, TNFRSF1A, TNFRSF1B, FAS, and FADD. In a specific embodiment, the biomarker is PERK. In a specific embodiment, the biomarker is EIF2a. In a specific embodiment, the biomarker is ATF4. In a specific embodiment, the biomarker is ATF3. In a specific embodiment, the biomarker is DDIT3. In a specific embodiment, the biomarker is PPP1R15A. In a specific embodiment, the biomarker is TNFRSF10B. In a specific embodiment, the biomarker is GADD45A. In a specific embodiment, the biomarker is TNFRSF1A. In a specific embodiment, the biomarker is TNFRSF1B. In a specific embodiment, the biomarker is FAS. In a specific embodiment, the biomarker is FADD.

As described in the Examples, the levels of the proteins in apoptosis pathway change in response Compound C treatment. Such proteins include Caspase 3, Caspase 7, Caspase 8, BID, Caspase 9, PARP, Mcl-1, and BAD. Thus, in some embodiments, the biomarker is selected from the group consisting of Caspase 3, Caspase 7, Caspase 8, BID, Caspase 9, PARP, Mcl-1, and BAD. In a specific embodiment, the biomarker is Caspase 3. In a specific embodiment, the biomarker is Caspase 7. In a specific embodiment, the biomarker is Caspase 8. In a specific embodiment, the biomarker is BID. In a specific embodiment, the biomarker is Caspase 9. In a specific embodiment, the biomarker is PARP. In a specific embodiment, the biomarker is Mcl-1. In yet another specific embodiment, the biomarker is BAD.

In other embodiments, the biomarker is a protein in XBP1 related pathway, such as IRE1, XBP1, SEC24D, DNAJB9, EDEM1, EDEM2, and HYOU1. Thus, in some embodiments, the biomarker is selected from the group consisting of IRE1, XBP1, SEC24D, DNAJB9, EDEM1, EDEM2, and HYOU1. In a specific embodiment, the biomarker is IRE1. In a specific embodiment, the biomarker is XBP1. In a specific embodiment, the biomarker is SEC24D. In a specific embodiment, the biomarker is DNAJB9. In a specific embodiment, the biomarker is EDEM1. In a specific embodiment, the biomarker is EDEM2. In a specific embodiment, the biomarker is HYOU1.

In yet other embodiments, the biomarker is a protein in ATF6 related pathway, such as ATF6, XBP1, EDEM1, EDEM2, HYOU1, and HSPA5. Thus, in some embodiments, the biomarker is selected from the group consisting of ATF6, XBP1, EDEM1, EDEM2, HYOU1, and HSPA5. In a specific embodiment, the biomarker is ATF6. In a specific embodiment, the biomarker is XBP1. In a specific embodiment, the biomarker is EDEM1. In a specific embodiment, the biomarker is EDEM2. In a specific embodiment, the biomarker is HYOU1. In a specific embodiment, the biomarker is HSPA5.

In some embodiments, the biomarker measured comprises one biomarker. In certain embodiments, the biomarkers measured comprise two biomarkers. In other embodiments, the biomarkers measured comprise three biomarkers. In certain embodiments, the biomarkers measured comprise four biomarkers. In some embodiments, the biomarkers measured comprise five biomarkers. In other embodiments, the biomarkers measured comprise six biomarkers. In yet other embodiments, the biomarkers measured comprise seven biomarkers. In certain embodiments, the biomarkers measured comprise eight biomarkers. In other embodiments, the biomarkers measured comprise nine biomarkers. In another embodiment, the biomarkers measured comprise ten or more biomarkers.

Also provided herein are methods for the treatment or management of cancer using a biomarker, e.g., eRF3a, eRF3b, eRF3c, ATF4, ATF3, or DDIT3, as a predictive or prognostic factor for the compounds provided herein. In certain embodiments, provided herein are methods for screening or identifying cancer patients, e.g., multiple myeloma, lymphoma, or leukemia patients, for treatment with a compound using the level of one or more biomarkers provided herein, e.g., eRF3a, eRF3b, eRF3c, ATF4, ATF3, or DDIT3, as a predictive or prognostic factor. In some embodiments, provided herein are methods for selecting patients having a higher response rate to therapy with a compound provided herein, using a biomarker (e.g., eRF3a, eRF3b, eRF3c, ATF4, ATF3, or DDIT3) level as a predictive or prognostic factor. In certain embodiments, the compound is Compound C.

In one aspect, provided herein is a method of identifying a subject having cancer who is likely to be responsive to a treatment compound, comprising:

(a) administering the treatment compound to the subject having the cancer;

(b) obtaining a sample from the subject;

(c) determining the level of a biomarker in the sample from the subject; and

(d) diagnosing the subject as being likely to be responsive to the treatment compound if the level of the biomarker in the sample of the subject changes as compared to a reference level of the biomarker;

wherein the treatment compound is a compound of Formula I:

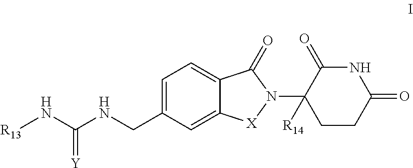

or a pharmaceutically acceptable salt, solvate, stereoisomer, isotopologue, prodrug, hydrate, co-crystal, clathrate, or a polymorph thereof, wherein:

X is CH₂ or C═O;

Y is O or S;

R¹³ is: (C₁-C₁₀)alkyl; (C₁-C₁₀)alkoxy; or 5 to 10 membered aryl or heteroaryl, optionally substituted with one or more of:

halogen; cyano; (C₁-C₆)alkylenedioxy; (C₁-C₆)alkoxy, itself optionally substituted with one or more halogen; (C₁-C₆)alkyl, itself optionally substituted with one or more halogen; or (C₁-C₆)alkylthio, itself optionally substituted with one or more halogen; and

R¹⁴ is H or (C₁-C₆)alkyl.

In another aspect, provided herein is a method of identifying a subject having cancer who is likely to be responsive to a treatment compound, comprising:

(a) obtaining a sample from the subject having the cancer;

(b) administering the treatment compound to the sample from the subject having the cancer;

(c) determining the level of a biomarker in the sample from the subject; and

(d) diagnosing the subject as being likely to be responsive to the treatment compound if the level of the biomarker in the sample of the subject changes as compared to a reference level of the biomarker;

wherein the treatment compound is a compound of Formula I:

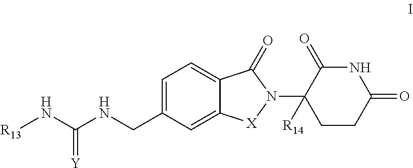

or a pharmaceutically acceptable salt, solvate, stereoisomer, isotopologue, prodrug, hydrate, co-crystal, clathrate, or a polymorph thereof, wherein:

X is CH₂ or C═O;

Y is O or S;

R¹³ is: (C₁-C₁₀)alkyl; (C₁-C₁₀)alkoxy; or 5 to 10 membered aryl or heteroaryl, optionally substituted with one or more of:

halogen; cyano; (C₁-C₆)alkylenedioxy; (C₁-C₆)alkoxy, itself optionally substituted with one or more halogen; (C₁-C₆)alkyl, itself optionally substituted with one or more halogen; or (C₁-C₆)alkylthio, itself optionally substituted with one or more halogen; and

R¹⁴ is H or (C₁-C₆)alkyl.

In some embodiments of the methods provided herein, administering a treatment compound to the sample from the subject having cancer is performed in vitro. In other embodiments, administering a treatment compound to the sample from the subject having cancer is performed in vivo. In one embodiment, the cells are contacted with the compound for a period of time, e.g., 5, 10, 15, 20, 25, 30, 35, 40, 45, 50, or 55 minutes, or 1, 2, 3, 4, 5, 6, 7, 8, 9, 10, 11, 12, 13, 14, 15, 16, 17, 18, 19, 20, 21, 22, 23, or 24 hours, or 2, 3, or more days. In other embodiments, the cells are obtained from a subject having (or suspected of having) cancer.

In some embodiments, the level of the biomarker in the sample of the subject is higher than the reference level of the biomarker.

In other embodiments, the level of the biomarker in the sample of the subject is lower than the reference level of the biomarker.

In another aspect, when a subject is diagnosed as being likely to be responsive to a treatment compound, the methods provided herein further comprise administering a therapeutically effective amount of the treatment compound to the subject diagnosed as being likely to be responsive to the treatment compound.

Thus, in some embodiments, provided herein is a method of treating cancer, comprising:

(a) obtaining a sample from a subject having the cancer;

(b) determining the level of a biomarker in the sample from the subject;

(c) diagnosing the subject as being likely to be responsive to a treatment compound if the level of the biomarker in the sample of the subject changes as compared to a reference level of the biomarker; and

(d) administering a therapeutically effective amount of the treatment compound to the subject diagnosed to be likely to be responsive to the treatment compound;

wherein the treatment compound is a compound of Formula I:

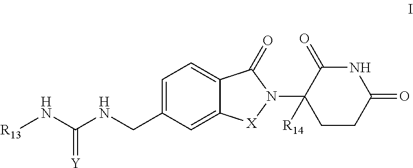

or a pharmaceutically acceptable salt, solvate, stereoisomer, isotopologue, prodrug, hydrate, co-crystal, clathrate, or a polymorph thereof, wherein:

X is CH₂ or C═O;

Y is O or S;

R¹³ is: (C₁-C₁₀)alkyl; (C₁-C₁₀)alkoxy; or 5 to 10 membered aryl or heteroaryl, optionally substituted with one or more of:

halogen; cyano; (C₁-C₆)alkylenedioxy; (C₁-C₆)alkoxy, itself optionally substituted with one or more halogen; (C₁-C₆)alkyl, itself optionally substituted with one or more halogen; or (C₁-C₆)alkylthio, itself optionally substituted with one or more halogen; and

R¹⁴ is H or (C₁-C₆)alkyl.

In some embodiments of the methods provided herein, administering a treatment compound to a subject having cancer is performed in vitro. In other embodiments, administering a treatment compound to a subject having cancer is performed in vivo. In one embodiment, the cells are contacted with the compound for a period of time, e.g., 5, 10, 15, 20, 25, 30, 35, 40, 45, 50, or 55 minutes, or 1, 2, 3, 4, 5, 6, 7, 8, 9, 10, 11, 12, 13, 14, 15, 16, 17, 18, 19, 20, 21, 22, 23, or 24 hours, or 2, 3, or more days. In other embodiments, the cells are obtained from a subject having (or suspected of having) the cancer.

In some embodiments, the level of the biomarker in the sample of the subject is higher than the reference level of the biomarker.

In other embodiments, the level of the biomarker in the sample of the subject is lower than the reference level of the biomarker.

In some embodiments of the various methods provided herein, a treatment compound is administered to a patient likely to be responsive to the treatment compound. Also provided herein are methods of treating patients who have been previously treated for cancer but are non-responsive to standard therapies, as well as those who have not previously been treated. The invention also encompasses methods of treating patients regardless of patient's age, although some diseases or disorders are more common in certain age groups. The invention further encompasses methods of treating patients who have undergone surgery in an attempt to treat the disease or condition at issue, as well as those who have not. Because patients with cancer have heterogeneous clinical manifestations and varying clinical outcomes, the treatment given to a patient may vary, depending on his/her prognosis. The skilled clinician will be able to readily determine without undue experimentation specific secondary agents, types of surgery, and types of non-drug based standard therapy that can be effectively used to treat an individual patient with cancer.

In certain embodiments, a therapeutically or prophylactically effective amount of the compound is from about 0.005 to about 1,000 mg per day, from about 0.01 to about 500 mg per day, from about 0.01 to about 250 mg per day, from about 0.01 to about 100 mg per day, from about 0.1 to about 100 mg per day, from about 0.5 to about 100 mg per day, from about 1 to about 100 mg per day, from about 0.01 to about 50 mg per day, from about 0.1 to about 50 mg per day, from about 0.5 to about 50 mg per day, from about 1 to about 50 mg per day, from about 0.02 to about 25 mg per day, or from about 0.05 to about 10 mg per day.

In certain embodiment, a therapeutically or prophylactically effective amount is from about 0.005 to about 1,000 mg per day, from about 0.01 to about 500 mg per day, from about 0.01 to about 250 mg per day, from about 0.01 to about 100 mg per day, from about 0.1 to about 100 mg per day, from about 0.5 to about 100 mg per day, from about 1 to about 100 mg per day, from about 0.01 to about 50 mg per day, from about 0.1 to about 50 mg per day, from about 0.5 to about 50 mg per day, from about 1 to about 50 mg per day, from about 0.02 to about 25 mg per day, or from about 0.05 to about 10 mg every other day.

In certain embodiments, the therapeutically or prophylactically effective amount is about 0.1, about 0.2, about 0.5, about 1, about 2, about 5, about 10, about 15, about 20, about 25, about 30, about 40, about 45, about 50, about 60, about 70, about 80, about 90, about 100, or about 150 mg per day.

In one embodiment, the recommended daily dose range of the compound of Formula I, or a pharmaceutically acceptable salt, solvate, stereoisomer, isotopologue, prodrug, hydrate, co-crystal, clathrate, or a polymorph thereof, for the conditions described herein lie within the range of from about 0.5 mg to about 50 mg per day, preferably given as a single once-a-day dose, or in divided doses throughout a day. In some embodiments, the dosage ranges from about 1 mg to about 50 mg per day. In other embodiments, the dosage ranges from about 0.5 mg to about 5 mg per day. Specific doses per day include 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 mg per day. In certain embodiments, the compound of Formula I is Compound C.

In a specific embodiment, the recommended starting dosage may be 0.5, 1, 2, 3, 4, 5, 10, 15, 20, 25, or 50 mg per day. In another embodiment, the recommended starting dosage may be 0.5, 1, 2, 3, 4, or 5 mg per day. The dose may be escalated to 15, 20, 25, 30, 35, 40, 45, or 50 mg/day. In a specific embodiment, the compound can be administered in an amount of about 25 mg/day to patients with leukemia, including AML. In a particular embodiment, the compound can be administered in an amount of about 10 mg/day to patients with leukemia, including AML.

In certain embodiments, the therapeutically or prophylactically effective amount is from about 0.001 to about 100 mg/kg/day, from about 0.01 to about 50 mg/kg/day, from about 0.01 to about 25 mg/kg/day, from about 0.01 to about 10 mg/kg/day, from about 0.01 to about 9 mg/kg/day, 0.01 to about 8 mg/kg/day, from about 0.01 to about 7 mg/kg/day, from about 0.01 to about 6 mg/kg/day, from about 0.01 to about 5 mg/kg/day, from about 0.01 to about 4 mg/kg/day, from about 0.01 to about 3 mg/kg/day, from about 0.01 to about 2 mg/kg/day, or from about 0.01 to about 1 mg/kg/day.

The administered dose can also be expressed in units other than mg/kg/day. For example, doses for parenteral administration can be expressed as mg/m2/day. One of ordinary skill in the art would readily know how to convert doses from mg/kg/day to mg/m2/day to given either the height or weight of a subject or both (see, www.fda.gov/cder/cancer/animalframe.htm). For example, a dose of 1 mg/kg/day for a 65 kg human is approximately equal to 38 mg/m2/day.

In certain embodiments, the amount of the compound administered is sufficient to provide a plasma concentration of the compound at steady state, ranging from about 0.001 to about 500 μM, about 0.002 to about 200 μM, about 0.005 to about 100 μM, about 0.01 to about 50 μM, from about 1 to about 50 μM, about 0.02 to about 25 μM, from about 0.05 to about 20 μM, from about 0.1 to about 20 μM, from about 0.5 to about 20 μM, or from about 1 to about 20 μM.

In other embodiments, the amount of the compound administered is sufficient to provide a plasma concentration of the compound at steady state, ranging from about 5 to about 100 nM, about 5 to about 50 nM, about 10 to about 100 nM, about 10 to about 50 nM, or from about 50 to about 100 nM.

As used herein, the term “plasma concentration at steady state” is the concentration reached after a period of administration of a compound provided herein, e.g., the compound of Formula I, or a pharmaceutically acceptable salt, solvate, stereoisomer, isotopologue, prodrug, hydrate, co-crystal, clathrate, or a polymorph thereof. Once steady state is reached, there are minor peaks and troughs on the time-dependent curve of the plasma concentration of the compound.

In certain embodiments, the amount of the compound administered is sufficient to provide a maximum plasma concentration (peak concentration) of the compound, ranging from about 0.001 to about 500 μM, about 0.002 to about 200 μM, about 0.005 to about 100 μM, about 0.01 to about 50 μM, from about 1 to about 50 μM, about 0.02 to about 25 μM, from about 0.05 to about 20 μM, from about 0.1 to about 20 μM, from about 0.5 to about 20 μM, or from about 1 to about 20 μM.

In certain embodiments, the amount of the compound administered is sufficient to provide a minimum plasma concentration (trough concentration) of the compound, ranging from about 0.001 to about 500 μM, about 0.002 to about 200 μM, about 0.005 to about 100 μM, about 0.01 to about 50 μM, from about 1 to about 50 μM, about 0.01 to about 25 μM, from about 0.01 to about 20 μM, from about 0.02 to about 20 μM, from about 0.02 to about 20 μM, or from about 0.01 to about 20 μM.

In certain embodiments, the amount of the compound administered is sufficient to provide an area under the curve (AUC) of the compound, ranging from about 100 to about 100,000 ng*hr/mL, from about 1,000 to about 50,000 ng*hr/mL, from about 5,000 to about 25,000 ng*hr/mL, or from about 5,000 to about 10,000 ng*hr/mL.

In certain embodiments, the patient to be treated with one of the methods provided herein has not been treated with anticancer therapy prior to the administration of the compound of Formula I, or a pharmaceutically acceptable salt, solvate, stereoisomer, isotopologue, prodrug, hydrate, co-crystal, clathrate, or a polymorph thereof. In certain embodiments, the patient to be treated with one of the methods provided herein has been treated with anticancer therapy prior to the administration of the compound of Formula I, or a pharmaceutically acceptable salt, solvate, stereoisomer, isotopologue, prodrug, hydrate, co-crystal, clathrate, or a polymorph thereof. In certain embodiments, the patient to be treated with one of the methods provided herein has developed drug resistance to the anticancer therapy. In certain embodiments, the compound of Formula I is Compound C.

Depending on the disease to be treated and the subject's condition, the compound of Formula I, or a pharmaceutically acceptable salt, solvate, stereoisomer, isotopologue, prodrug, hydrate, co-crystal, clathrate, or a polymorph thereof, may be administered by oral, parenteral (e.g., intramuscular, intraperitoneal, intravenous, CIV, intracistemal injection or infusion, subcutaneous injection, or implant), inhalation, nasal, vaginal, rectal, sublingual, or topical (e.g., transdermal or local) routes of administration. The compound of Formula I, or a pharmaceutically acceptable salt, solvate, stereoisomer, isotopologue, prodrug, hydrate, co-crystal, clathrate, or a polymorph thereof, may be formulated, alone or together, in suitable dosage unit with pharmaceutically acceptable excipients, carriers, adjuvants, and vehicles, appropriate for each route of administration. In certain embodiments, the compound of Formula I is Compound C.

In one embodiment, the compound of Formula I, or a pharmaceutically acceptable salt, solvate, stereoisomer, isotopologue, prodrug, hydrate, co-crystal, clathrate, or a polymorph thereof, is administered orally. In another embodiment, the compound of Formula I, or a pharmaceutically acceptable salt, solvate, stereoisomer, isotopologue, prodrug, hydrate, co-crystal, clathrate, or a polymorph thereof, is administered parenterally. In yet another embodiment, the compound of Formula I, or a pharmaceutically acceptable salt, solvate, stereoisomer, isotopologue, prodrug, hydrate, co-crystal, clathrate, or a polymorph thereof, is administered intravenously. In certain embodiments, the compound of Formula I is Compound C.

The compound of Formula I, or a pharmaceutically acceptable salt, solvate, stereoisomer, isotopologue, prodrug, hydrate, co-crystal, clathrate, or a polymorph thereof, can be delivered as a single dose (e.g., a single bolus injection or an oral tablet or pill), or over time (e.g., continuous infusion over time or divided bolus doses over time). The compound can be administered repeatedly if necessary, for example, until the patient experiences stable disease or regression, or until the patient experiences disease progression or unacceptable toxicity. For example, stable disease for solid cancers generally means that the perpendicular diameter of measurable lesions has not increased by 25% or more from the last measurement. Therasse et al., J. Natl. Cancer Inst. 2000, 92(3):205-216. Stable disease or lack thereof is determined by methods known in the art such as evaluation of patient symptoms, physical examination, and visualization of the tumor that has been imaged using X-ray, CAT, PET, MRI scan, or other commonly accepted evaluation modalities. In certain embodiments, the compound of Formula I is Compound C.

The compound of Formula I, or a pharmaceutically acceptable salt, solvate, stereoisomer, isotopologue, prodrug, hydrate, co-crystal, clathrate, or a polymorph thereof, can be administered once daily (QD) or divided into multiple daily doses such as twice daily (BID), three times daily (TID), and four times daily (QID). In addition, the administration can be continuous (i.e., daily for consecutive days or every day) or intermittent, e.g., in cycles (i.e., including days, weeks, or months of rest without drug). As used herein, the term “daily” is intended to mean that a therapeutic compound, such as the compound of Formula I, or a pharmaceutically acceptable salt, solvate, stereoisomer, isotopologue, prodrug, hydrate, co-crystal, clathrate, or a polymorph thereof, is administered once or more than once each day, for example, for a period of time. The term “continuous” is intended to mean that a therapeutic compound, such as the compound of Formula I, or a pharmaceutically acceptable salt, solvate, stereoisomer, isotopologue, prodrug, hydrate, co-crystal, clathrate, or a polymorph thereof, is administered daily for an uninterrupted period of at least 10 days to 52 weeks. The term “intermittent” or “intermittently” as used herein is intended to mean stopping and starting at either regular or irregular intervals. For example, intermittent administration of the compound of Formula I, or a pharmaceutically acceptable salt, solvate, stereoisomer, isotopologue, prodrug, hydrate, co-crystal, clathrate, or a polymorph thereof, is administration for one to six days per week, administration in cycles (e.g., daily administration for two to eight consecutive weeks, then a rest period with no administration for up to one week), or administration on alternate days. The term “cycling” as used herein is intended to mean that a therapeutic compound, such as the compound of Formula I, or a pharmaceutically acceptable salt, solvate, stereoisomer, isotopologue, prodrug, hydrate, co-crystal, clathrate, or a polymorph thereof, is administered daily or continuously but with a rest period. In certain embodiments, the rest period is the same length as the treatment period. In other embodiments, the rest period has different length from the treatment period. In certain embodiments, the compound of Formula I is Compound C.

In some embodiments, the frequency of administration is in the range of about a daily dose to about a monthly dose. In certain embodiments, administration is once a day, twice a day, three times a day, four times a day, once every other day, twice a week, once every week, once every two weeks, once every three weeks, or once every four weeks. In one embodiment, the compound of Formula I, or a pharmaceutically acceptable salt, solvate, stereoisomer, isotopologue, prodrug, hydrate, co-crystal, clathrate, or a polymorph thereof, is administered once a day. In another embodiment, the compound of Formula I, or a pharmaceutically acceptable salt, solvate, stereoisomer, isotopologue, prodrug, hydrate, co-crystal, clathrate, or a polymorph thereof, is administered twice a day. In yet another embodiment, the compound of Formula I, or a pharmaceutically acceptable salt, solvate, stereoisomer, isotopologue, prodrug, hydrate, co-crystal, clathrate, or a polymorph thereof, is administered three times a day. In still another embodiment, the compound of Formula I, or a pharmaceutically acceptable salt, solvate, stereoisomer, isotopologue, prodrug, hydrate, co-crystal, clathrate, or a polymorph thereof, is administered four times a day. In certain embodiments, the compound of Formula I is Compound C.

In certain embodiments, the compound of Formula I, or a pharmaceutically acceptable salt, solvate, stereoisomer, isotopologue, prodrug, hydrate, co-crystal, clathrate, or a polymorph thereof, is administered once per day from one day to six months, from one week to three months, from one week to four weeks, from one week to three weeks, or from one week to two weeks. In certain embodiments, the compound of Formula I, or a pharmaceutically acceptable salt, solvate, stereoisomer, isotopologue, prodrug, hydrate, co-crystal, clathrate, or a polymorph thereof, is administered once per day for one week, two weeks, three weeks, or four weeks. In one embodiment, the compound of Formula I, or a pharmaceutically acceptable salt, solvate, stereoisomer, isotopologue, prodrug, hydrate, co-crystal, clathrate, or a polymorph thereof, is administered once per day for one week. In another embodiment, the compound of Formula I, or a pharmaceutically acceptable salt, solvate, stereoisomer, isotopologue, prodrug, hydrate, co-crystal, clathrate, or a polymorph thereof, is administered once per day for two weeks. In yet another embodiment, the compound of Formula I, or a pharmaceutically acceptable salt, solvate, stereoisomer, isotopologue, prodrug, hydrate, co-crystal, clathrate, or a polymorph thereof, is administered once per day for three weeks. In still another embodiment, the compound of Formula I, or a pharmaceutically acceptable salt, solvate, stereoisomer, isotopologue, prodrug, hydrate, co-crystal, clathrate, or a polymorph thereof, is administered once per day for four weeks. In certain embodiments, the compound of Formula I is Compound C.

Also provided herein are methods for predicting or monitoring the responsiveness of a patient to a treatment compound, or efficacy of a treatment compound, using a biomarker (e.g., eRF3a, eRF3b, eRF3c, ATF4, ATF3, or DDIT3). In certain embodiments, provided herein are methods for predicting the responsiveness of a subject having or suspected of having cancer (e.g., multiple myeloma, lymphoma, or leukemia), to a treatment compound, using a predictive or prognostic factor, such as eRF3a, eRF3b, eRF3c, ATF4, ATF3, or DDIT3 level. In some embodiments, provided herein are methods for monitoring the efficacy of a treatment of cancer (e.g., multiple myeloma, lymphoma, or leukemia) in a subject with a treatment compound using a biomarker (e.g., eRF3a, eRF3b, eRF3c, ATF4, ATF3, or DDIT3) level as a predictive or prognostic factor. In certain embodiments, the compound is Compound C.

Thus, in yet another aspect, provided herein is a method of predicting the responsiveness of a subject having or suspected of having cancer to a treatment compound, comprising:

(a) administering the treatment compound to the subject having the cancer;

(b) obtaining a sample from the subject;

(c) determining the level of a biomarker in the sample from the subject;

(d) diagnosing the subject as being likely to be responsive to a treatment of the cancer with the treatment compound if the level of the biomarker in the sample changes as compared to the level of the biomarker obtained from a reference sample;

wherein the treatment compound is a compound of Formula I:

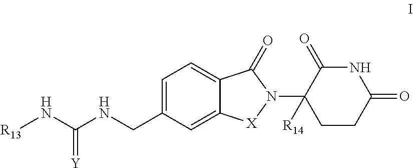

or a pharmaceutically acceptable salt, solvate, stereoisomer, isotopologue, prodrug, hydrate, co-crystal, clathrate, or a polymorph thereof, wherein:

X is CH₂ or C═O;

Y is O or S;

R¹³ is: (C₁-C₁₀)alkyl; (C₁-C₁₀)alkoxy; or 5 to 10 membered aryl or heteroaryl, optionally substituted with one or more of:

halogen; cyano; (C₁-C₆)alkylenedioxy; (C₁-C₆)alkoxy, itself optionally substituted with one or more halogen; (C₁-C₆)alkyl, itself optionally substituted with one or more halogen; or (C₁-C₆)alkylthio, itself optionally substituted with one or more halogen; and

R¹⁴ is H or (C₁-C₆)alkyl.

In yet another aspect, provided herein is a method of predicting the responsiveness of a subject having or suspected of having cancer to a treatment compound, comprising:

(a) obtaining a sample from the subject having the cancer;

(b) administering the treatment compound to the sample from the subject having the cancer;

(c) determining the level of a biomarker in the sample from the subject;

(d) diagnosing the subject as being likely to be responsive to a treatment of the cancer with the treatment compound if the level of the biomarker in the sample changes as compared to the level of the biomarker obtained from a reference sample;

wherein the treatment compound is a compound of Formula I:

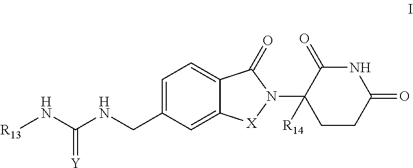

or a pharmaceutically acceptable salt, solvate, stereoisomer, isotopologue, prodrug, hydrate, co-crystal, clathrate, or a polymorph thereof, wherein:

X is CH₂ or C═O;

Y is O or S;

R¹³ is: (C₁-C₁₀)alkyl; (C₁-C₁₀)alkoxy; or 5 to 10 membered aryl or heteroaryl, optionally substituted with one or more of:

halogen; cyano; (C₁-C₆)alkylenedioxy; (C₁-C₆)alkoxy, itself optionally substituted with one or more halogen; (C₁-C₆)alkyl, itself optionally substituted with one or more halogen; or (C₁-C₆)alkylthio, itself optionally substituted with one or more halogen; and

R¹⁴ is H or (C₁-C₆)alkyl.

In some embodiments of the methods provided herein, administering the treatment compound to the sample from the subject having cancer is performed in vitro. In other embodiments, administering the treatment compound to the sample from the subject having cancer is performed in vivo. In one embodiment, the cells are contacted with the compound for a period of time, e.g., 5, 10, 15, 20, 25, 30, 35, 40, 45, 50, or 55 minutes, or 1, 2, 3, 4, 5, 6, 7, 8, 9, 10, 11, 12, 13, 14, 15, 16, 17, 18, 19, 20, 21, 22, 23, or 24 hours, or 2, 3, or more days. In other embodiments, the cells are obtained from a subject having (or suspected of having) cancer.

In some embodiments of the various methods provided herein, the level of the biomarker in the sample is higher than the level of the biomarker obtained from the reference sample.

In other embodiments of the various methods provided herein, the level of the biomarker in the sample is lower than the level of the biomarker obtained from the reference sample.

In yet another aspect, provided herein is a method of monitoring the efficacy of a treatment of cancer in a subject with a treatment compound, comprising:

(a) administering the treatment compound to the subject having the cancer;

(b) obtaining a sample from the subject;

(c) determining the level of a biomarker in the sample from the subject;

(d) comparing the level of the biomarker in the sample with the level of the biomarker obtained from a reference sample, wherein a change in the level as compared to the reference is indicative of the efficacy of the treatment compound in treating the cancer in the subject;

wherein the treatment compound is a compound of Formula I:

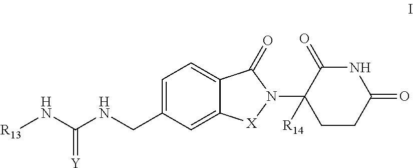

or a pharmaceutically acceptable salt, solvate, stereoisomer, isotopologue, prodrug, hydrate, co-crystal, clathrate, or a polymorph thereof, wherein:

X is CH₂ or C═O;

Y is O or S;

R¹³ is: (C₁-C₁₀)alkyl; (C₁-C₁₀)alkoxy; or 5 to 10 membered aryl or heteroaryl, optionally substituted with one or more of:

halogen; cyano; (C₁-C₆)alkylenedioxy; (C₁-C₆)alkoxy, itself optionally substituted with one or more halogen; (C₁-C₆)alkyl, itself optionally substituted with one or more halogen; or (C₁-C₆)alkylthio, itself optionally substituted with one or more halogen; and

R¹⁴ is H or (C₁-C₆)alkyl.

In some embodiments, an increased level as compared to the reference is indicative of the efficacy of the treatment compound in treating the cancer in the subject.

In other embodiments, a decreased level as compared to the reference is indicative of the efficacy of the treatment compound in treating the cancer in the subject.

In some embodiments of the various methods provided herein, the method further comprises administering a therapeutically effective amount of a second active agent or a support care therapy. The second active agents can be large molecules (e.g., proteins) or small molecules (e.g., synthetic inorganic, organometallic, or organic molecules). In some embodiments, the second active agent is a hematopoietic growth factor, cytokine, anti-cancer agent, antibiotic, cox-2 inhibitor, immunomodulatory agent, immunosuppressive agent, corticosteroid, therapeutic antibody that specifically binds to a cancer antigen or a pharmacologically active mutant, or derivative thereof.

In some embodiments, the second active agents are small molecules that can alleviate adverse effects associated with the administration of a compound provided herein, or a pharmaceutically acceptable salt, solvate, stereoisomer, isotopologue, prodrug, hydrate, co-crystal, clathrate, or a polymorph thereof. Many small molecule second active agents are believed to be capable of providing a synergistic effect when administered with (e.g., before, after, or simultaneously) a compound provided herein, or a pharmaceutically acceptable salt, solvate, stereoisomer, isotopologue, prodrug, hydrate, co-crystal, clathrate, or a polymorph thereof. Examples of small molecule second active agents include, but are not limited to, anti-cancer agents, antibiotics, immunosuppressive agents, and steroids.

In some embodiments of the various methods provided herein, the reference is prepared by using a control sample obtained from the subject prior to administering the treatment compound to the subject, and the control sample is from the same source as the sample. In other embodiments of the various methods provided herein, the reference is prepared by using a control sample obtained from a healthy subject not having cancer, and the control sample is from the same source as the sample.

In some embodiments of the various methods provided herein, the cancer is solid cancer or blood borne cancer. In some embodiments, the cancer is solid cancer. In some embodiments, the solid cancer is metastatic. In some embodiments, the solid cancer is hepatocellular carcinoma, melanoma, prostate cancer, ovarian cancer, or glioblastoma. In some embodiments, the cancer is blood borne tumor. In certain embodiments, the blood borne tumor is metastatic. In some embodiments of the various methods provided herein, the cancer is MM. In certain embodiments, the cancer is leukemia. The cancers provided herein include various types of leukemia such as CLL, CML, ALL, or AML. In a specific embodiment, the leukemia is AML. In a specific embodiment, the leukemia is relapsed, refractory, or resistant to conventional therapies. In certain embodiments, the cancer provided here is lymphoma, including but not limited to NHL. In some embodiments, the cancer provided herein is NHL, including but not limited to DLBCL.

In some embodiments, methods provided herein encompass treating, preventing, or managing various types of cancers. In one embodiment, methods provided herein encompass treating, preventing, or managing various types of leukemia such as CLL, CML, ALL, or AML by administering a therapeutically effective amount of a compound of Formula I, or a pharmaceutically acceptable salt, solvate, stereoisomer, isotopologue, prodrug, hydrate, co-crystal, clathrate, or a polymorph thereof. In certain embodiments, the compound of Formula I is Compound C.

In some embodiments, the methods provided herein encompass treating, preventing, or managing acute leukemia in a subject. In some embodiments, the acute leukemia is AML, which includes, but is not limited to, undifferentiated AML (M0), myeloblastic leukemia (M1), myeloblastic leukemia (M2), promyelocytic leukemia (M3 or M3 variant [M3V]), myelomonocytic leukemia (M4 or M4 variant with eosinophilia [M4E]), monocytic leukemia (M5), erythroleukemia (M6), and megakaryoblastic leukemia (M7). In one embodiment, the acute myeloid leukemia is undifferentiated AML (M0). In one embodiment, the acute myeloid leukemia is myeloblastic leukemia (M1). In one embodiment, the acute myeloid leukemia is myeloblastic leukemia (M2). In one embodiment, the acute myeloid leukemia is promyelocytic leukemia (M3 or M3 variant [M3V]). In one embodiment, the acute myeloid leukemia is myelomonocytic leukemia (M4 or M4 variant with eosinophilia [M4E]). In one embodiment, the acute myeloid leukemia is monocytic leukemia (M5). In one embodiment, the acute myeloid leukemia is erythroleukemia (M6). In one embodiment, the acute myeloid leukemia is megakaryoblastic leukemia (M7). Thus, the methods of treating, preventing, or managing AML in a subject comprise the step of administering to the subject an amount of a compound provided herein or an enantiomer or a mixture of enantiomers thereof, or a pharmaceutically acceptable salt, solvate, hydrate, co-crystal, clathrate, or polymorph thereof, effective to treat, prevent, or manage acute myeloid leukemia alone or in combination with a second active agent. In some embodiments, the methods comprise the step of administering to the subject a compound provided herein, or a pharmaceutically acceptable salt, solvate, stereoisomer, isotopologue, prodrug, hydrate, co-crystal, clathrate, or a polymorph thereof, in combination with a second active agent in amounts effective to treat, prevent, or manage AML.

In some embodiments, the methods provided herein encompass treating, preventing, or managing ALL in a subject. In some embodiments, ALL includes leukemia that originates in the blast cells of the bone marrow (B-cells), thymus (T-cells), and lymph nodes. The acute lymphocytic leukemia can be categorized according to the French-American-British (FAB) Morphological Classification Scheme as L1—Mature-appearing lymphoblasts (T-cells or pre-B-cells), L2—Immature and pleomorphic (variously shaped) lymphoblasts (T-cells or pre-B-cells), and L3—Lymphoblasts (B-cells or Burkitt's cells). In one embodiment, the ALL originates in the blast cells of the bone marrow (B-cells). In one embodiment, the ALL originates in the thymus (T-cells). In one embodiment, the ALL originates in the lymph nodes. In one embodiment, the ALL is L1 type characterized by mature-appearing lymphoblasts (T-cells or pre-B-cells). In one embodiment, the ALL is L2 type characterized by immature and pleomorphic (variously shaped) lymphoblasts (T-cells or pre-B-cells). In one embodiment, the ALL is L3 type characterized by lymphoblasts (B-cells or Burkitt's cells). In certain embodiments, the ALL is T-cell leukemia. In one embodiment, the T-cell leukemia is peripheral T-cell leukemia. In another embodiment, the T-cell leukemia is T-cell lymphoblastic leukemia. In another embodiment, the T-cell leukemia is cutaneous T-cell leukemia. In another embodiment, the T-cell leukemia is adult T-cell leukemia. Thus, the methods of treating, preventing, or managing ALL in a subject comprise the step of administering to the subject an amount of a compound provided herein, or a pharmaceutically acceptable salt, solvate, stereoisomer, isotopologue, prodrug, hydrate, co-crystal, clathrate, or a polymorph thereof, effective to treat, prevent, or manage ALL alone or in combination with a second active agent. In some embodiments, the methods comprise the step of administering to the subject a compound provided herein, or a pharmaceutically acceptable salt, solvate, stereoisomer, isotopologue, prodrug, hydrate, co-crystal, clathrate, or a polymorph thereof, in combination with a second active agent in amounts effective to treat, prevent, or manage ALL.

In some embodiments, the methods provided herein encompass treating, preventing, or managing CML in a subject. The methods comprise the step of administering to the subject an amount of a compound provided herein, or a pharmaceutically acceptable salt, solvate, stereoisomer, isotopologue, prodrug, hydrate, co-crystal, clathrate, or a polymorph thereof, effective to treat, prevent, or manage chronic myelogenous leukemia alone or in combination with a second active agent. In some embodiments, the methods comprise the step of administering to the subject a compound provided herein, or a pharmaceutically acceptable salt, solvate, stereoisomer, isotopologue, prodrug, hydrate, co-crystal, clathrate, or a polymorph thereof, in combination with a second active agent in amounts effective to treat, prevent, or manage CML.

In some embodiments, the methods provided herein encompass treating, preventing, or managing CLL in a subject. The methods comprise the step of administering to the subject an amount of a compound provided herein, or a pharmaceutically acceptable salt, solvate, stereoisomer, isotopologue, prodrug, hydrate, co-crystal, clathrate, or a polymorph thereof, effective to treat, prevent, or manage chronic lymphocytic leukemia alone or in combination with a second active agent. In some embodiments, the methods comprise the step of administering to the subject a compound provided herein, or a pharmaceutically acceptable salt, solvate, stereoisomer, isotopologue, prodrug, hydrate, co-crystal, clathrate, or a polymorph thereof, in combination with a second active agent in amounts effective to treat, prevent, or manage CLL.

In certain embodiments, provided herein are methods of treating, preventing, or managing lymphoma, including NHL, comprising administering a therapeutically effective amount of the compound of Formula I, or a pharmaceutically acceptable salt, solvate, stereoisomer, isotopologue, prodrug, hydrate, co-crystal, clathrate, or a polymorph thereof, or an enantiomer or a mixture of enantiomers thereof, or a pharmaceutically acceptable salt, solvate, hydrate, stereoisomer, tautomer or racemic mixtures thereof, to a patient having lymphoma alone or in combination with a second active agent. In some embodiments, the methods comprise the step of administering to the subject a compound provided herein, or a pharmaceutically acceptable salt, solvate, stereoisomer, isotopologue, prodrug, hydrate, co-crystal, clathrate, or a polymorph thereof, in combination with a second active agent in amounts effective to treat, prevent, or manage lymphoma. In some embodiments, provided herein are methods for the treatment or management of NHL, including but not limited to DLBCL. In certain embodiments, the compound of Formula I is Compound C.

In certain embodiments, provided herein are methods of treating, preventing, or managing disease in patients with impaired renal function. In certain embodiments, provided herein are method of treating, preventing, or managing cancer in patients with impaired renal function. In certain embodiments, provided herein are methods of providing appropriate dose adjustments for patients with impaired renal function due to, but not limited to, disease, aging, or other patient factors.

In certain embodiments, provided herein are methods of treating, preventing, or managing MM, including relapsed/refractory MM in patients with impaired renal function or a symptom thereof, comprising administering a therapeutically effective amount of the compound of Formula I, or a pharmaceutically acceptable salt, solvate, stereoisomer, isotopologue, prodrug, hydrate, co-crystal, clathrate, or a polymorph thereof, or an enantiomer or a mixture of enantiomers thereof, or a pharmaceutically acceptable salt, solvate, hydrate, stereoisomer, tautomer or racemic mixtures thereof, to a patient having relapsed/refractory MM with impaired renal function alone or in combination with a second active agent. In some embodiments, the methods comprise the step of administering to the subject a compound provided herein, or a pharmaceutically acceptable salt, solvate, stereoisomer, isotopologue, prodrug, hydrate, co-crystal, clathrate, or a polymorph thereof, in combination with a second active agent in amounts effective to treat, prevent, or manage relapsed/refractory MM in patients with impaired renal function. In certain embodiments, the compound of Formula I is Compound C.

In some embodiments of the various methods provided herein, the biomarker provided herein is selected from the group consisting of ATF3, ATF4, ATF6, BAD, BID, BIP, Caspase 3, Caspase 7, Caspase 8, Caspase 9, CK1a, DDIT3, DNAJB9, EDEM1, EDEM2, eEF1α, EIF2a, FADD, FAS, GADD45A, HSPA5, HYOU1, IKZF1, IKZF3, IRE1, Mcl-1, PABP1, PARP, PERK, PPP1R15A, eRF1, eRF3a, eRF3b, eRF3c, SEC24D, TNFRSF1A, TNFRSF1B, TNFRSF10B, and XBP1. In some embodiments of the various methods provided herein, the biomarker is selected from the group consisting of eRF3a, eRF3b, eRF3c, ATF4, ATF3, and DDIT3.

In a specific embodiment, the biomarker is eRF3a. In a specific embodiment, the biomarker is eRF3b. In a specific embodiment, the biomarker is eRF3c. In a specific embodiment, the biomarker is IKZF1. In a specific embodiment, the biomarker is IKZF3. In a specific embodiment, the biomarker is CK1a. In a specific embodiment, the biomarker is PABP1. In a specific embodiment, the biomarker is eRF1. In a specific embodiment, the biomarker is BIP. In a specific embodiment, the biomarker is unmodified BIP. In a specific embodiment, the biomarker is C-terminal modified BIP. In a specific embodiment, the biomarker is C-terminal modified BIP that cannot be recognized by KDEL antibody. In a specific embodiment, the biomarker is C-terminal modified BIP that cannot be recognized by BIP antibody that recognizes unmodified C-terminus of BIP. In a specific embodiment, the biomarker is C-terminal modified BIP that cannot be recognized by both KDEL antibody and BIP antibody that recognizes unmodified C-terminus of BIP. In a specific embodiment, the biomarker is eEF1α. In a specific embodiment, the biomarker is PERK. In a specific embodiment, the biomarker is unphosphorylated PERK. In a specific embodiment, the biomarker is phosphorylated PERK. In a specific embodiment, the biomarker is EIF2a. In a specific embodiment, the biomarker is unphosphorylatd EIF2a. In a specific embodiment, the biomarker is phosphorylatd EIF2a. In a specific embodiment, the biomarker is ATF4. In a specific embodiment, the biomarker is ATF3. In a specific embodiment, the biomarker is the splicing variant of ATF3. In a specific embodiment, the biomarker is DDIT3. In a specific embodiment, the biomarker is PPP1R15A. In a specific embodiment, the biomarker is TNFRSF10B. In a specific embodiment, the biomarker is GADD45A. In a specific embodiment, the biomarker is TNFRSF1A. In a specific embodiment, the biomarker is TNFRSF1B. In a specific embodiment, the biomarker is FAS. In a specific embodiment, the biomarker is FADD. In a specific embodiment, the biomarker is IRE1. In a specific embodiment, the biomarker is unphosphorylated IRE1. In a specific embodiment, the biomarker is phosphorylated IRE1. In a specific embodiment, the biomarker is XBP1. In a specific embodiment, the biomarker is SEC24D. In a specific embodiment, the biomarker is DNAJB9. In a specific embodiment, the biomarker is EDEM1. In a specific embodiment, the biomarker is EDEM2. In a specific embodiment, the biomarker is HYOU1. In a specific embodiment, the biomarker is ATF6. In a specific embodiment, the biomarker is HSPA5. In a specific embodiment, the biomarker is Caspase 8. In a specific embodiment, the biomarker is cleaved Caspase 8. In a specific embodiment, the biomarker is BID. In a specific embodiment, the biomarker is Caspase 9. In a specific embodiment, the biomarker is cleaved Caspase 9. In a specific embodiment, the biomarker is Caspase 3. In a specific embodiment, the biomarker is cleaved Caspase 3. In a specific embodiment, the biomarker is PARP. In a specific embodiment, the biomarker is Caspase 7. In a specific embodiment, the biomarker is cleaved Caspase 7. In a specific embodiment, the biomarker is Mcl-1. In yet another specific embodiment, the biomarker is BAD. In a specific embodiment, the biomarker is unphosphorylated BAD. In a specific embodiment, the biomarker is phosphorylated BAD (e.g., pS112-BAD).

In some embodiments, the level of the biomarker decreases in response to the compound treatment. In some embodiments, the biomarker is selected from the group consisting of eRF3a, eRF3b, eRF3c, eRF1, IKZF1, IKZF3, CK1a, BIP, Mcl-1, and BAD, and the level of the biomarker decreases as compared to a reference in response to a treatment compound.

In other embodiments the level of the biomarker increases in response to the compound treatment. In some embodiments, the biomarker is selected from the group consisting of ATF4, ATF3, and DDIT3, and the level of the biomarker increases as compared to a reference in response to a treatment compound. In other embodiments, the biomarker is selected from the group consisting of SEC24D, DNAJB9, XBP1, EDEM1, EDEM2, HYOU1, EIF2a, PPP1R15A, GADD45A, TNFRSF1B, TNFRSF10B, cleaved form of Caspase 8, BID, cleaved form of Caspase 9, cleaved form of Caspase 3, cleaved form of Caspase 7, cleaved PARP, FAS, and FADD, and the level of the biomarker increases in response to the compound treatment.

In certain embodiments of the various methods provided herein, the biomarker is a protein that is directly or indirectly affected by CRBN, for example through protein-protein interactions (e.g., certain CRBN substrates or downstream effectors thereof), or through various cellular pathways (e.g., signal transduction pathways). In specific embodiments, the biomarker is a CRBN-associated protein (CAP). In some embodiments, the biomarker is mRNA of a protein that is directly or indirectly affected by CRBN. In other embodiments, the biomarker is cDNA of a protein that is directly or indirectly affected by CRBN.

Thus, in some embodiments, provided herein is a method of identifying a subject having cancer who is likely to be responsive to a treatment compound, comprising:

(a) administering the treatment compound to the subject having the cancer;

(b) obtaining a sample from the subject;

(c) determining the level of a biomarker in the sample from the subject, wherein the biomarker is a CAP,

(d) diagnosing the subject as being likely to be responsive to the treatment compound if the level of the biomarker in the sample of the subject changes as compared to a reference level;

wherein the treatment compound is a compound of Formula I:

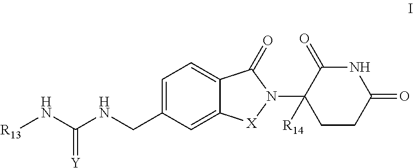

or a pharmaceutically acceptable salt, solvate, stereoisomer, isotopologue, prodrug, hydrate, co-crystal, clathrate, or a polymorph thereof, wherein:

X is CH₂ or C═O;

Y is O or S;

R¹³ is: (C₁-C₁₀)alkyl; (C₁-C₁₀)alkoxy; or 5 to 10 membered aryl or heteroaryl, optionally substituted with one or more of:

halogen; cyano; (C₁-C₆)alkylenedioxy; (C₁-C₆)alkoxy, itself optionally substituted with one or more halogen; (C₁-C₆)alkyl, itself optionally substituted with one or more halogen; or (C₁-C₆)alkylthio, itself optionally substituted with one or more halogen; and

R¹⁴ is H or (C₁-C₆)alkyl.

In some embodiments, provided herein is a method of identifying a subject having a cancer who is likely to be responsive to a treatment compound, comprising:

(a) obtaining a sample from the subject having the cancer;

(b) administering the treatment compound to the sample from the subject having the cancer;

(c) determining the level of a biomarker in the sample from the subject, wherein the biomarker is a CAP,

(d) diagnosing the subject as being likely to be responsive to the treatment compound if the level of the biomarker in the sample of the subject changes as compared to a reference level;

wherein the treatment compound is a compound of Formula I:

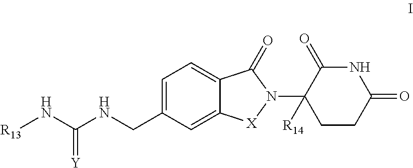

or a pharmaceutically acceptable salt, solvate, stereoisomer, isotopologue, prodrug, hydrate, co-crystal, clathrate, or a polymorph thereof, wherein:

X is CH₂ or C═O;

Y is O or S;

R¹³ is: (C₁-C₁₀)alkyl; (C₁-C₁₀)alkoxy; or 5 to 10 membered aryl or heteroaryl, optionally substituted with one or more of:

halogen; cyano; (C₁-C₆)alkylenedioxy; (C₁-C₆)alkoxy, itself optionally substituted with one or more halogen; (C₁-C₆)alkyl, itself optionally substituted with one or more halogen; or (C₁-C₆)alkylthio, itself optionally substituted with one or more halogen; and

R¹⁴ is H or (C₁-C₆)alkyl.

In some embodiments, provided herein is a method of treating cancer, comprising:

(a) obtaining a sample from a subject having the cancer;

(b) determining the level of a biomarker in the sample from the subject, wherein the biomarker is CAP;

(c) diagnosing the subject as being likely to be responsive to a treatment compound if the level of the biomarker in the sample of the subject changes as compared to a reference level; and

(d) administering a therapeutically effective amount of the treatment compound to the subject diagnosed to be likely to be responsive to the treatment compound;

wherein the treatment compound is a compound of Formula I:

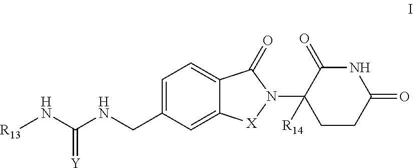

or a pharmaceutically acceptable salt, solvate, stereoisomer, isotopologue, prodrug, hydrate, co-crystal, clathrate, or a polymorph thereof, wherein:

X is CH₂ or C═O;

Y is O or S;

R¹³ is: (C₁-C₁₀)alkyl; (C₁-C₁₀)alkoxy; or 5 to 10 membered aryl or heteroaryl, optionally substituted with one or more of:

halogen; cyano; (C₁-C₆)alkylenedioxy; (C₁-C₆)alkoxy, itself optionally substituted with one or more halogen; (C₁-C₆)alkyl, itself optionally substituted with one or more halogen; or (C₁-C₆)alkylthio, itself optionally substituted with one or more halogen; and

R¹⁴ is H or (C₁-C₆)alkyl.

In some embodiments, provided herein is a method of predicting the responsiveness of a subject having or suspected of having cancer to a treatment compound, comprising:

(a) administering the treatment compound to the subject having the cancer;

(b) obtaining a sample from the subject;

(c) determining the level of a biomarker in the sample from the subject, wherein the biomarker is a CAP;

(d) diagnosing the subject as being likely to be responsive to a treatment of the cancer with the treatment compound if the level of the biomarker in the sample changes as compared to the level of the biomarker obtained from a reference sample;

wherein the treatment compound is a compound of Formula I:

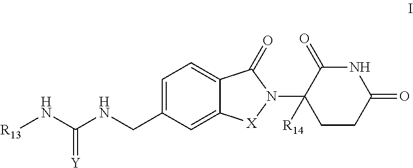

or a pharmaceutically acceptable salt, solvate, stereoisomer, isotopologue, prodrug, hydrate, co-crystal, clathrate, or a polymorph thereof, wherein:

X is CH₂ or C═O;

Y is O or S;

R¹³ is: (C₁-C₁₀)alkyl; (C₁-C₁₀)alkoxy; or 5 to 10 membered aryl or heteroaryl, optionally substituted with one or more of:

halogen; cyano; (C₁-C₆)alkylenedioxy; (C₁-C₆)alkoxy, itself optionally substituted with one or more halogen; (C₁-C₆)alkyl, itself optionally substituted with one or more halogen; or (C₁-C₆)alkylthio, itself optionally substituted with one or more halogen; and

R¹⁴ is H or (C₁-C₆)alkyl.

In some embodiments, provided herein is a method of predicting the responsiveness of a subject having or suspected of having cancer to a treatment compound, comprising:

(a) obtaining a sample from the subject having the cancer;

(b) administering the treatment compound to the sample from the subject having the cancer;

(c) determining the level of a biomarker in the sample from the subject, wherein the biomarker is a CAP;

(d) diagnosing the subject as being likely to be responsive to a treatment of the cancer with the treatment compound if the level of the biomarker in the sample decreases as compared to the level of the biomarker obtained from a reference sample;

wherein the treatment compound is a compound of Formula I:

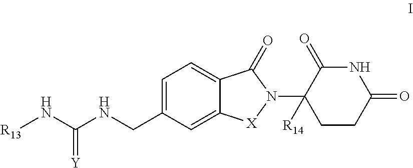

or a pharmaceutically acceptable salt, solvate, stereoisomer, isotopologue, prodrug, hydrate, co-crystal, clathrate, or a polymorph thereof, wherein:

X is CH₂ or C═O;

Y is O or S;

R¹³ is: (C₁-C₁₀)alkyl; (C₁-C₁₀)alkoxy; or 5 to 10 membered aryl or heteroaryl, optionally substituted with one or more of:

halogen; cyano; (C₁-C₆)alkylenedioxy; (C₁-C₆)alkoxy, itself optionally substituted with one or more halogen; (C₁-C₆)alkyl, itself optionally substituted with one or more halogen; or (C₁-C₆)alkylthio, itself optionally substituted with one or more halogen; and

R¹⁴ is H or (C₁-C₆)alkyl.

In some embodiments, provided herein is a method of monitoring the efficacy of a treatment of cancer in a subject with a treatment compound, comprising:

(a) administering the treatment compound to the subject having the cancer;

(b) obtaining a sample from the subject;

(c) determining the level of the biomarker in the sample from the subject, wherein the biomarker is a CAP;

(d) comparing the level of the biomarker in the sample with the level of the biomarker obtained from a reference sample, wherein a changed level as compared to the reference is indicative of the efficacy of the treatment compound in treating the cancer in the subject;

wherein the treatment compound is a compound of Formula I:

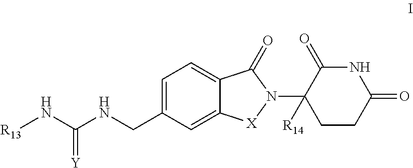

or a pharmaceutically acceptable salt, solvate, stereoisomer, isotopologue, prodrug, hydrate, co-crystal, clathrate, or a polymorph thereof, wherein:

X is CH₂ or C═O;

Y is O or S;

R¹³ is: (C₁-C₁₀)alkyl; (C₁-C₁₀)alkoxy; or 5 to 10 membered aryl or heteroaryl, optionally substituted with one or more of:

halogen; cyano; (C₁-C₆)alkylenedioxy; (C₁-C₆)alkoxy, itself optionally substituted with one or more halogen; (C₁-C₆)alkyl, itself optionally substituted with one or more halogen; or (C₁-C₆)alkylthio, itself optionally substituted with one or more halogen; and

R¹⁴ is H or (C₁-C₆)alkyl.

In some embodiments, the CAP is selected from the group consisting of eRF3a, eRF3b, eRF3c, IKZF1, IKZF3, and CK1a. In some embodiments, the biomarker is an eRF3 family member, such as eRF3a, eRF3b, and eRF3c. In a specific embodiment, the biomarker is eRF3a. In another specific embodiment, the biomarker is eRF3b. In yet another specific embodiment, the biomarker is eRF3c. In yet another specific embodiment, the biomarker is IKZF1. In yet another embodiment, the biomarker is IKZF3. In yet another embodiment, the biomarker is CK1a. In other embodiments, the biomarker is a binding partner of, downstream effector of, or a factor in a cellular pathway impacted by eRF3a, eRF3b, eRF3c, IKZF1, IKZF3, and CK1a. For example, in some embodiments, the biomarker is a binding partner of, downstream effector of, or a factor in a cellular pathway impacted by an eRF3 family member. In a specific embodiment, the biomarker is a binding partner of eRF3a, such as eRF1.

As shown in the Examples, the level of an eRF3 family member, such as eRF3a, eRF3b, or eRF3c, decreases as compared to a reference in response to Compound C treatment. Accordingly, in some embodiments, the biomarker is an eRF3 family member, such as eRF3a, eRF3b, and eRF3c, and the level of the biomarker decreases in response to the Compound C treatment. Thus, in some embodiments of the various methods provided herein, the biomarker is eRF3a, eRF3b, eRF3c or a protein (or a factor) impacted thereby, and wherein the level of the biomarker decreases as compared to a reference.

In some embodiments, provided herein is a method of identifying a subject having cancer who is likely to be responsive to a treatment compound, comprising:

(a) administering the treatment compound to the subject having the cancer;

(b) obtaining a sample from the subject;

(c) determining the level of eRF3a, eRF3b, or eRF3c in the sample from the subject,

(d) diagnosing the subject as being likely to be responsive to the treatment compound if the level of eRF3a, eRF3b, or eRF3c in the sample of the subject decreases as compared to a reference level;

wherein the treatment compound is a compound of Formula I:

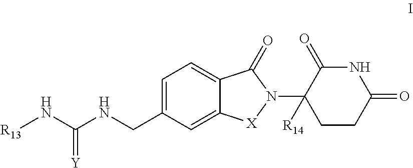

or a pharmaceutically acceptable salt, solvate, stereoisomer, isotopologue, prodrug, hydrate, co-crystal, clathrate, or a polymorph thereof, wherein:

X is CH₂ or C═O;

Y is O or S;

R¹³ is: (C₁-C₁₀)alkyl; (C₁-C₁₀)alkoxy; or 5 to 10 membered aryl or heteroaryl, optionally substituted with one or more of:

halogen; cyano; (C₁-C₆)alkylenedioxy; (C₁-C₆)alkoxy, itself optionally substituted with one or more halogen; (C₁-C₆)alkyl, itself optionally substituted with one or more halogen; or (C₁-C₆)alkylthio, itself optionally substituted with one or more halogen; and

R¹⁴ is H or (C₁-C₆)alkyl.

In some embodiments, provided herein is a method of identifying a subject having a cancer who is likely to be responsive to a treatment compound, comprising:

(a) obtaining a sample from the subject having the cancer;

(b) administering the treatment compound to the sample from the subject having the cancer;

(c) determining the level of eRF3a, eRF3b, or eRF3c in the sample from the subject,

(d) diagnosing the subject as being likely to be responsive to the treatment compound if the level of eRF3a, eRF3b, or eRF3c in the sample of the subject decreases as compared to a reference level;

wherein the treatment compound is a compound of Formula I:

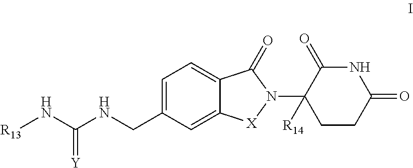

or a pharmaceutically acceptable salt, solvate, stereoisomer, isotopologue, prodrug, hydrate, co-crystal, clathrate, or a polymorph thereof, wherein:

X is CH₂ or C═O;

Y is O or S;

R¹³ is: (C₁-C₁₀)alkyl; (C₁-C₁₀)alkoxy; or 5 to 10 membered aryl or heteroaryl, optionally substituted with one or more of:

halogen; cyano; (C₁-C₆)alkylenedioxy; (C₁-C₆)alkoxy, itself optionally substituted with one or more halogen; (C₁-C₆)alkyl, itself optionally substituted with one or more halogen; or (C₁-C₆)alkylthio, itself optionally substituted with one or more halogen; and

R¹⁴ is H or (C₁-C₆)alkyl.

In some embodiments, provided herein is a method of treating cancer, comprising:

(a) obtaining a sample from a subject having the cancer;

(b) determining the level of eRF3a, eRF3b, or eRF3c in the sample from the subject;

(c) diagnosing the subject as being likely to be responsive to a treatment compound if the level of eRF3a, eRF3b, or eRF3c in the sample of the subject decreases as compared to a reference level; and

(d) administering a therapeutically effective amount of the treatment compound to the subject diagnosed to be likely to be responsive to the treatment compound;

wherein the treatment compound is a compound of Formula I:

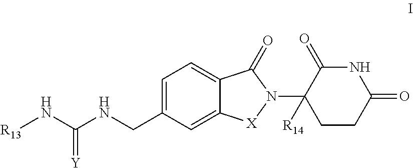

or a pharmaceutically acceptable salt, solvate, stereoisomer, isotopologue, prodrug, hydrate, co-crystal, clathrate, or a polymorph thereof, wherein:

X is CH₂ or C═O;

Y is O or S;

R¹³ is: (C₁-C₁₀)alkyl; (C₁-C₁₀)alkoxy; or 5 to 10 membered aryl or heteroaryl, optionally substituted with one or more of:

halogen; cyano; (C₁-C₆)alkylenedioxy; (C₁-C₆)alkoxy, itself optionally substituted with one or more halogen; (C₁-C₆)alkyl, itself optionally substituted with one or more halogen; or (C₁-C₆)alkylthio, itself optionally substituted with one or more halogen; and

R¹⁴ is H or (C₁-C₆)alkyl.

In some embodiments, provided herein is a method of predicting the responsiveness of a subject having or suspected of having cancer to a treatment compound, comprising:

(a) administering the treatment compound to the subject having the cancer;

(b) obtaining a sample from the subject;

(c) determining the level of eRF3a, eRF3b, or eRF3c in the sample from the subject;

(d) diagnosing the subject as being likely to be responsive to a treatment of the cancer with the treatment compound if the level of eRF3a, eRF3b, or eRF3c in the sample decreases as compared to the level of eRF3a, eRF3b, or eRF3c obtained from a reference sample;

wherein the treatment compound is a compound of Formula I:

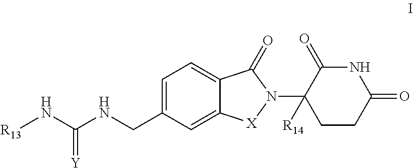

or a pharmaceutically acceptable salt, solvate, stereoisomer, isotopologue, prodrug, hydrate, co-crystal, clathrate, or a polymorph thereof, wherein:

X is CH₂ or C═O;

Y is O or S;

R¹³ is: (C₁-C₁₀)alkyl; (C₁-C₁₀)alkoxy; or 5 to 10 membered aryl or heteroaryl, optionally substituted with one or more of:

halogen; cyano; (C₁-C₆)alkylenedioxy; (C₁-C₆)alkoxy, itself optionally substituted with one or more halogen; (C₁-C₆)alkyl, itself optionally substituted with one or more halogen; or (C₁-C₆)alkylthio, itself optionally substituted with one or more halogen; and

R¹⁴ is H or (C₁-C₆)alkyl.

In some embodiments, provided herein is a method of predicting the responsiveness of a subject having or suspected of having cancer to a treatment compound, comprising:

(a) obtaining a sample from the subject having the cancer;

(b) administering the treatment compound to the sample from the subject having the cancer;

(c) determining the level of eRF3a, eRF3b, or eRF3c in the sample from the subject;

(d) diagnosing the subject as being likely to be responsive to a treatment of the cancer with the treatment compound if the level of eRF3a, eRF3b, or eRF3c in the sample decreases as compared to the level of eRF3a, eRF3b, or eRF3c obtained from a reference sample;

wherein the treatment compound is a compound of Formula I:

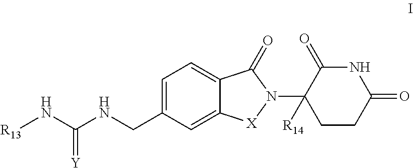

or a pharmaceutically acceptable salt, solvate, stereoisomer, isotopologue, prodrug, hydrate, co-crystal, clathrate, or a polymorph thereof, wherein:

X is CH₂ or C═O;

Y is O or S;

R¹³ is: (C₁-C₁₀)alkyl; (C₁-C₁₀)alkoxy; or 5 to 10 membered aryl or heteroaryl, optionally substituted with one or more of:

halogen; cyano; (C₁-C₆)alkylenedioxy; (C₁-C₆)alkoxy, itself optionally substituted with one or more halogen; (C₁-C₆)alkyl, itself optionally substituted with one or more halogen; or (C₁-C₆)alkylthio, itself optionally substituted with one or more halogen; and

R¹⁴ is H or (C₁-C₆)alkyl.

In some embodiments, provided herein is a method of monitoring the efficacy of a treatment of cancer in a subject with a treatment compound, comprising:

(a) administering the treatment compound to the subject having the cancer;

(b) obtaining a sample from the subject;

(c) determining the level of eRF3a, eRF3b, or eRF3c in the sample from the subject;

(d) comparing the level of eRF3a, eRF3b, or eRF3c in the sample with the level of eRF3a, eRF3b, or eRF3c obtained from a reference sample, wherein a decreased level as compared to the reference is indicative of the efficacy of the treatment compound in treating the cancer in the subject;

wherein the treatment compound is a compound of Formula I:

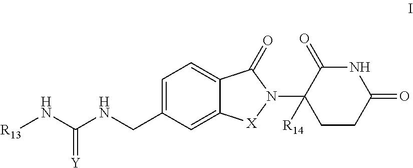

or a pharmaceutically acceptable salt, solvate, stereoisomer, isotopologue, prodrug, hydrate, co-crystal, clathrate, or a polymorph thereof, wherein:

X is CH₂ or C═O;

Y is O or S;

R¹³ is: (C₁-C₁₀)alkyl; (C₁-C₁₀)alkoxy; or 5 to 10 membered aryl or heteroaryl, optionally substituted with one or more of:

halogen; cyano; (C₁-C₆)alkylenedioxy; (C₁-C₆)alkoxy, itself optionally substituted with one or more halogen; (C₁-C₆)alkyl, itself optionally substituted with one or more halogen; or (C₁-C₆)alkylthio, itself optionally substituted with one or more halogen; and

R¹⁴ is H or (C₁-C₆)alkyl.

In a specific embodiment, the biomarker is eRF3a, and the cancer is MM, lymphoma, or leukemia. In one embodiment, the cancer is MM. In a specific embodiment, the cancer is lymphoma. In some embodiments, the cancer is leukemia. In another specific embodiment, the leukemia is CLL, CML, ALL, or AML. In one embodiment, the leukemia is AML. In a specific embodiment, the biomarker is eRF3a, and the treatment compound is Compound C.

In another specific embodiment, the biomarker is eRF3b, and the cancer is MM, lymphoma, or leukemia. In one embodiment, the cancer is MM. In a specific embodiment, the cancer is lymphoma. In some embodiments, the cancer is leukemia. In another specific embodiment, the leukemia is CLL, CML, ALL, or AML. In one embodiment, the leukemia is AML. In another specific embodiment, the biomarker is eRF3b, and the treatment compound is Compound C.

In another specific embodiment, the biomarker is eRF3c, and the cancer is MM, lymphoma, or leukemia. In one embodiment, the cancer is MM. In a specific embodiment, the cancer is lymphoma. In some embodiments, the cancer is leukemia. In another specific embodiment, the leukemia is CLL, CML, ALL, or AML. In one embodiment, the leukemia is AML. In another specific embodiment, the biomarker is eRF3c, and the treatment compound is Compound C.

Downregulation of these eRF3 family members result in protein misfolding and/or aggregation, protein mislocation, and direct change of protein function, among other effects. One cellular pathway affected is unfolded protein response (UPR), which is a cellular stress response related to the endoplasmic reticulum (ER). Thus, a factor or a protein involved in UPR or a downstream pathway thereof can be used as a biomarker according to the present disclosure. The pathways related to UPR include, but not limited to, PERK related signaling pathway and related apoptosis pathway, XBP1 related signaling pathway, and ATF6 related signaling pathway. Thus, in some embodiments, the biomarker provided herein has a function in ER stress pathway. In some embodiments, the biomarker provided herein has a function in UPR pathway. In certain embodiments, the biomarker provided herein has a function in PERK related signaling pathway. In other embodiments, the biomarker provided herein has a function in XBP1 related signaling pathway. In yet other embodiments, the biomarker provided herein has a function in ATF6 related signaling pathway. In some embodiments, the biomarker provided herein has a function in FAS/FADD signaling and apoptosis pathway.

For example, as shown in the Examples, the levels of proteins in PERK related signaling pathway change in response to Compound C treatment, such as PERK, EIF2a, ATF4, ATF3, DDIT3, PPP1R15A, TNFRSF10B, GADD45A, TNFRSF1A, TNFRSF1B, FAS, and FADD. Thus, in some embodiments, the biomarker provided herein is selected from the group consisting of PERK, EIF2a, ATF4, ATF3, DDIT3, PPP1R15A, TNFRSF10B, GADD45A, TNFRSF1A, TNFRSF1B, FAS, and FADD. In a specific embodiment, the biomarker is PERK. In a specific embodiment, the biomarker is EIF2a. In a specific embodiment, the biomarker is ATF4. In a specific embodiment, the biomarker is ATF3. In a specific embodiment, the biomarker is DDIT3. In a specific embodiment, the biomarker is PPP1R15A. In a specific embodiment, the biomarker is TNFRSF10B. In a specific embodiment, the biomarker is GADD45A. In a specific embodiment, the biomarker is TNFRSF1A. In a specific embodiment, the biomarker is TNFRSF1B. In a specific embodiment, the biomarker is FAS. In a specific embodiment, the biomarker is FADD.

In other embodiments of the various methods provided herein, the biomarker is selected from a group of factors having a function in PERK related signaling pathway. In some embodiments, a biomarker involved in PERK related signaling pathway is used for identifying a subject having cancer who is likely to be responsive to a treatment compound; predicting the responsiveness of a subject having or suspected of having cancer to a treatment compound; monitoring the efficacy of a treatment of cancer in a subject with a treatment compound; or treating cancer.

In some more specific embodiments, the biomarker involved in PERK related signaling pathway is selected from the group consisting of ATF4, ATF3, or DDIT3, and wherein the level of the biomarker increases as compared to a reference. Thus, in some embodiments, provided herein is a method of identifying a subject having cancer who is likely to be responsive to a treatment compound, comprising:

(a) administering the treatment compound to the subject having the cancer;

(b) obtaining a sample from the subject;

(c) determining the level of ATF4, ATF3, or DDIT3 in the sample from the subject,

(d) diagnosing the subject as being likely to be responsive to the treatment compound if the level of ATF4, ATF3, or DDIT3 in the sample of the subject increases as compared to a reference level;

wherein the treatment compound is a compound of Formula I:

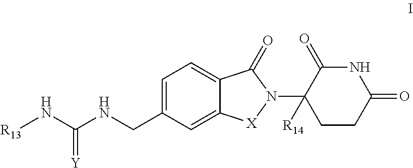

or a pharmaceutically acceptable salt, solvate, stereoisomer, isotopologue, prodrug, hydrate, co-crystal, clathrate, or a polymorph thereof, wherein:

X is CH₂ or C═O;

Y is O or S;

R¹³ is: (C₁-C₁₀)alkyl; (C₁-C₁₀)alkoxy; or 5 to 10 membered aryl or heteroaryl, optionally substituted with one or more of:

halogen; cyano; (C₁-C₆)alkylenedioxy; (C₁-C₆)alkoxy, itself optionally substituted with one or more halogen; (C₁-C₆)alkyl, itself optionally substituted with one or more halogen; or (C₁-C₆)alkylthio, itself optionally substituted with one or more halogen; and

R¹⁴ is H or (C₁-C₆)alkyl.

In some embodiments, provided herein is a method of identifying a subject having cancer who is likely to be responsive to a treatment compound, comprising:

(a) obtaining a sample from the subject having the cancer;

(b) administering the treatment compound to the sample from the subject having the cancer;

(c) determining the level of ATF4, ATF3, or DDIT3 in the sample from the subject,

(d) diagnosing the subject as being likely to be responsive to the treatment compound if the level of ATF4, ATF3, or DDIT3 in the sample of the subject increases as compared to a reference level;

wherein the treatment compound is a compound of Formula I:

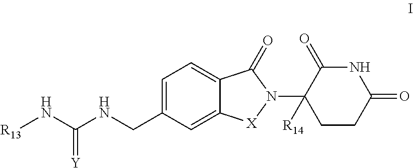

or a pharmaceutically acceptable salt, solvate, stereoisomer, isotopologue, prodrug, hydrate, co-crystal, clathrate, or a polymorph thereof, wherein:

X is CH₂ or C═O;

Y is O or S;

R¹³ is: (C₁-C₁₀)alkyl; (C₁-C₁₀)alkoxy; or 5 to 10 membered aryl or heteroaryl, optionally substituted with one or more of:

halogen; cyano; (C₁-C₆)alkylenedioxy; (C₁-C₆)alkoxy, itself optionally substituted with one or more halogen; (C₁-C₆)alkyl, itself optionally substituted with one or more halogen; or (C₁-C₆)alkylthio, itself optionally substituted with one or more halogen; and

R¹⁴ is H or (C₁-C₆)alkyl.

In some embodiments, provided herein is a method of treating cancer, comprising:

(a) obtaining a sample from a subject having the cancer;

(b) determining the level of ATF4, ATF3, or DDIT3 in the sample from the subject;

(c) diagnosing the subject as being likely to be responsive to a treatment compound if the level of ATF4, ATF3, or DDIT3 in the sample of the subject increases as compared to a reference level; and

(d) administering a therapeutically effective amount of the treatment compound to the subject diagnosed to be likely to be responsive to the treatment compound;

wherein the treatment compound is a compound of Formula I:

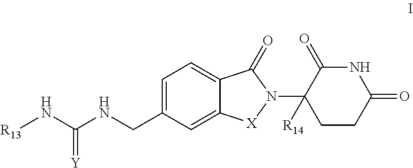

or a pharmaceutically acceptable salt, solvate, stereoisomer, isotopologue, prodrug, hydrate, co-crystal, clathrate, or a polymorph thereof, wherein:

X is CH₂ or C═O;

Y is O or S;

R¹³ is: (C₁-C₁₀)alkyl; (C₁-C₁₀)alkoxy; or 5 to 10 membered aryl or heteroaryl, optionally substituted with one or more of:

halogen; cyano; (C₁-C₆)alkylenedioxy; (C₁-C₆)alkoxy, itself optionally substituted with one or more halogen; (C₁-C₆)alkyl, itself optionally substituted with one or more halogen; or (C₁-C₆)alkylthio, itself optionally substituted with one or more halogen; and

R¹⁴ is H or (C₁-C₆)alkyl.

In some embodiments, provided herein is a method of predicting the responsiveness of a subject having or suspected of having cancer to a treatment compound, comprising:

(a) administering the treatment compound to the subject having the cancer;

(b) obtaining a sample from the subject;

(c) determining the level of ATF4, ATF3, or DDIT3 in the sample from the subject;

(d) diagnosing the subject as being likely to be responsive to a treatment of the cancer with the treatment compound if the level of ATF4, ATF3, or DDIT3 in the sample increases as compared to the level of ATF4, ATF3, or DDIT3 obtained from a reference sample;

wherein the treatment compound is a compound of Formula I:

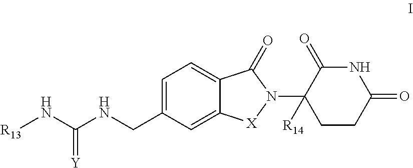

or a pharmaceutically acceptable salt, solvate, stereoisomer, isotopologue, prodrug, hydrate, co-crystal, clathrate, or a polymorph thereof, wherein:

X is CH₂ or C═O;

Y is O or S;

R¹³ is: (C₁-C₁₀)alkyl; (C₁-C₁₀)alkoxy; or 5 to 10 membered aryl or heteroaryl, optionally substituted with one or more of:

halogen; cyano; (C₁-C₆)alkylenedioxy; (C₁-C₆)alkoxy, itself optionally substituted with one or more halogen; (C₁-C₆)alkyl, itself optionally substituted with one or more halogen; or (C₁-C₆)alkylthio, itself optionally substituted with one or more halogen; and

R¹⁴ is H or (C₁-C₆)alkyl.

In some embodiments, provided herein is a method of predicting the responsiveness of a subject having or suspected of having cancer to a treatment compound, comprising:

(a) obtaining a sample from the subject having the cancer;

(b) administering the treatment compound to the sample from the subject having the cancer;

(c) determining the level of ATF4, ATF3, or DDIT3 in the sample from the subject;

(d) diagnosing the subject as being likely to be responsive to a treatment of the cancer with the treatment compound if the level of ATF4, ATF3, or DDIT3 in the sample increases as compared to the level of ATF4, ATF3, or DDIT3 obtained from a reference sample;

wherein the treatment compound is a compound of Formula I:

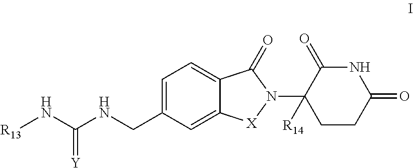

or a pharmaceutically acceptable salt, solvate, stereoisomer, isotopologue, prodrug, hydrate, co-crystal, clathrate, or a polymorph thereof, wherein:

X is CH₂ or C═O;

Y is O or S;

R¹³ is: (C₁-C₁₀)alkyl; (C₁-C₁₀)alkoxy; or 5 to 10 membered aryl or heteroaryl, optionally substituted with one or more of:

halogen; cyano; (C₁-C₆)alkylenedioxy; (C₁-C₆)alkoxy, itself optionally substituted with one or more halogen; (C₁-C₆)alkyl, itself optionally substituted with one or more halogen; or (C₁-C₆)alkylthio, itself optionally substituted with one or more halogen; and

R¹⁴ is H or (C₁-C₆)alkyl.

In some embodiments, provided herein is a method of monitoring the efficacy of a treatment of cancer in a subject with a treatment compound, comprising:

(a) administering the treatment compound to the subject having the cancer;

(b) obtaining a sample from the subject;

(c) determining the level of ATF4, ATF3, or DDIT3 in the sample from the subject;

(d) comparing the level of ATF4, ATF3, or DDIT3 in the sample with the level of ATF4, ATF3, or DDIT3 obtained from a reference sample, wherein increased level as compared to the reference is indicative of the efficacy of the treatment compound in treating the cancer in the subject;

wherein the treatment compound is a compound of Formula I:

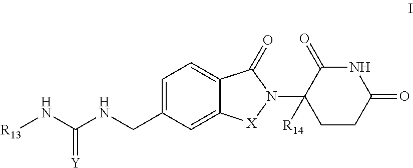

or a pharmaceutically acceptable salt, solvate, stereoisomer, isotopologue, prodrug, hydrate, co-crystal, clathrate, or a polymorph thereof, wherein:

X is CH₂ or C═O;

Y is O or S;

R¹³ is: (C₁-C₁₀)alkyl; (C₁-C₁₀)alkoxy; or 5 to 10 membered aryl or heteroaryl, optionally substituted with one or more of:

halogen; cyano; (C₁-C₆)alkylenedioxy; (C₁-C₆)alkoxy, itself optionally substituted with one or more halogen; (C₁-C₆)alkyl, itself optionally substituted with one or more halogen; or (C₁-C₆)alkylthio, itself optionally substituted with one or more halogen; and

R¹⁴ is H or (C₁-C₆)alkyl.

In one specific embodiment, the biomarker is ATF4, and the cancer is MM, lymphoma, or leukemia. In one embodiment, the cancer is MM. In a specific embodiment, the cancer is lymphoma. In some embodiments, the cancer is leukemia. In another specific embodiment, the leukemia is CLL, CML, ALL, or AML. In one embodiment, the leukemia is AML. In a specific embodiment, the biomarker is ATF4, and the treatment compound is Compound C.

In a specific embodiment, the biomarker is ATF3, and the cancer is MM, lymphoma, or leukemia. In one embodiment, the cancer is MM. In a specific embodiment, the cancer is lymphoma. In some embodiments, the cancer is leukemia. In another specific embodiment, the leukemia is CLL, CML, ALL, or AML. In one embodiment, the leukemia is AML. In a specific embodiment, the biomarker is ATF3, and the treatment compound is Compound C.

In another specific embodiment, the biomarker is DDIT3, and the cancer is MM, lymphoma, or leukemia. In one embodiment, the cancer is MM. In a specific embodiment, the cancer is lymphoma. In some embodiments, the cancer is leukemia. In another specific embodiment, the leukemia is CLL, CML, ALL, or AML. In one embodiment, the leukemia is AML. In a specific embodiment, the biomarker is DDIT3, and the treatment compound is Compound C.

In other embodiments, a biomarker has a function in apoptosis pathway. In some embodiments, the biomarker is selected from the group consisting of Caspase 3, Caspase 7, Caspase 8, BID, Caspase 9, PARP, Mcl-1, and pS112-BAD. In a specific embodiment, the biomarker is Caspase 3. In a specific embodiment, the biomarker is Caspase 7. In a specific embodiment, the biomarker is Caspase 8. In a specific embodiment, the biomarker is BID. In a specific embodiment, the biomarker is Caspase 9. In a specific embodiment, the biomarker is PARP. In a specific embodiment, the biomarker is Mcl-1. In yet another specific embodiment, the biomarker is pS112-BAD.

In some embodiments, a biomarker involved in apoptosis pathway is used for identifying a subject having cancer who is likely to be responsive to a treatment compound; predicting the responsiveness of a subject having or suspected of having cancer to a treatment compound; monitoring the efficacy of a treatment of cancer in a subject with a treatment compound; or treating cancer.

In other embodiments, the biomarker has a function in XBP1 related pathway, such as IRE1, XBP1, SEC24D, DNAJB9, EDEM1, EDEM2, and HYOU1. Thus, in some embodiments, the biomarker is selected from the group consisting of IRE1, XBP1, SEC24D, DNAJB9, EDEM1, EDEM2, and HYOU1. In a specific embodiment, the biomarker is IRE1. In a specific embodiment, the biomarker is XBP1. In a specific embodiment, the biomarker is SEC24D. In a specific embodiment, the biomarker is DNAJB9. In a specific embodiment, the biomarker is EDEM1. In a specific embodiment, the biomarker is EDEM2. In a specific embodiment, the biomarker is HYOU1. In some embodiments, a biomarker involved in XBP1 related pathway is used for identifying a subject having cancer who is likely to be responsive to a treatment compound; predicting the responsiveness of a subject having or suspected of having cancer to a treatment compound; monitoring the efficacy of a treatment of cancer in a subject with a treatment compound; or treating cancer.

In yet other embodiments, the biomarker is a protein in ATF6 related pathway, such as ATF6, XBP1, EDEM1, EDEM2, HYOU1, and HSPA5. Thus, in some embodiments, the biomarker is selected from the group consisting of ATF6, XBP1, EDEM1, EDEM2, HYOU1, and HSPA5. In a specific embodiment, the biomarker is ATF6. In a specific embodiment, the biomarker is XBP1. In a specific embodiment, the biomarker is EDEM1. In a specific embodiment, the biomarker is EDEM2. In a specific embodiment, the biomarker is HYOU1. In a specific embodiment, the biomarker is HSPA5. In some embodiments, a biomarker involved in ATF6 related pathway is used for identifying a subject having cancer who is likely to be responsive to a treatment compound; predicting the responsiveness of a subject having or suspected of having cancer to a treatment compound; monitoring the efficacy of a treatment of cancer in a subject with a treatment compound; or treating cancer.

In some embodiments of the various methods provided herein, the level of the biomarkers is measured by determining the protein level of the biomarker. In some embodiments, the methods provided herein comprise contacting proteins within the sample with a first antibody that immunospecifically binds to the biomarker protein. In some embodiments, the methods provided herein further comprise (i) contacting the biomarker protein bound to the first antibody with a second antibody with a detectable label, wherein the second antibody immunospecifically binds to the biomarker protein, and wherein the second antibody immunospecifically binds to a different epitope on the biomarker protein than the first antibody; (ii) detecting the presence of the second antibody bound to the biomarker protein; and (iii) determining the amount of the biomarker protein based on the amount of detectable label in the second antibody. In other embodiments, the methods provided herein further comprises (i) contacting the biomarker protein bound to the first antibody with a second antibody with a detectable label, wherein the second antibody immunospecifically binds to the first antibody; (ii) detecting the presence of the second antibody bound to the first antibody; and (iii) determining the amount of the biomarker protein based on the amount of detectable label in the second antibody.

In other embodiments of the various methods provided herein, the level of the biomarkers is measured by determining the mRNA level of the biomarker.

In yet other embodiments of the various methods provided herein, the level of the biomarkers is measured by determining the cDNA level of the biomarker.

In some embodiments of the various methods provided herein, the treatment compound is a compound described in Section 5.7 below.

In some embodiments of the various methods provided herein, the treatment compound is of Formula I:

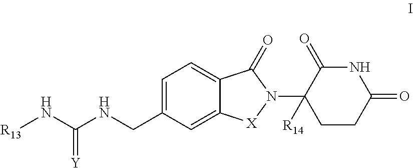

or a pharmaceutically acceptable salt, solvate, stereoisomer, isotopologue, prodrug, hydrate, co-crystal, clathrate, or a polymorph thereof, wherein:

X is CH₂ or C═O;

Y is O or S;

R¹³ is: (C₁-C₁₀)alkyl; (C₁-C₁₀)alkoxy; or 5 to 10 membered aryl or heteroaryl, optionally substituted with one or more of:

halogen; cyano; (C₁-C₆)alkylenedioxy; (C₁-C₆)alkoxy, itself optionally substituted with one or more halogen; (C₁-C₆)alkyl, itself optionally substituted with one or more halogen;

or (C₁-C₆)alkylthio, itself optionally substituted with one or more halogen; and

R¹⁴ is H or (C₁-C₆)alkyl.

In some embodiments, the treatment compound is selected from a group consisting of:

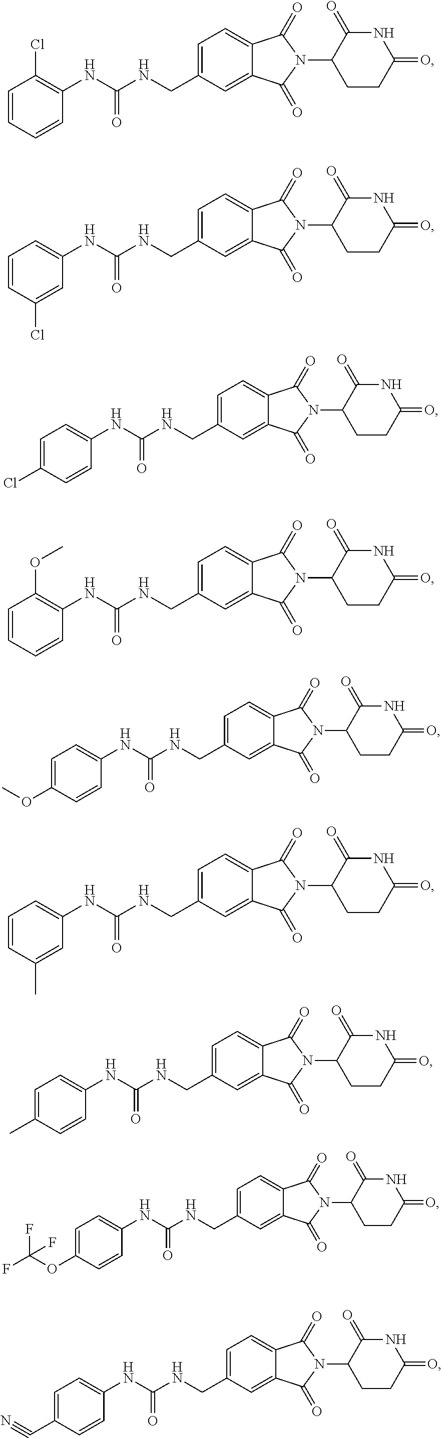

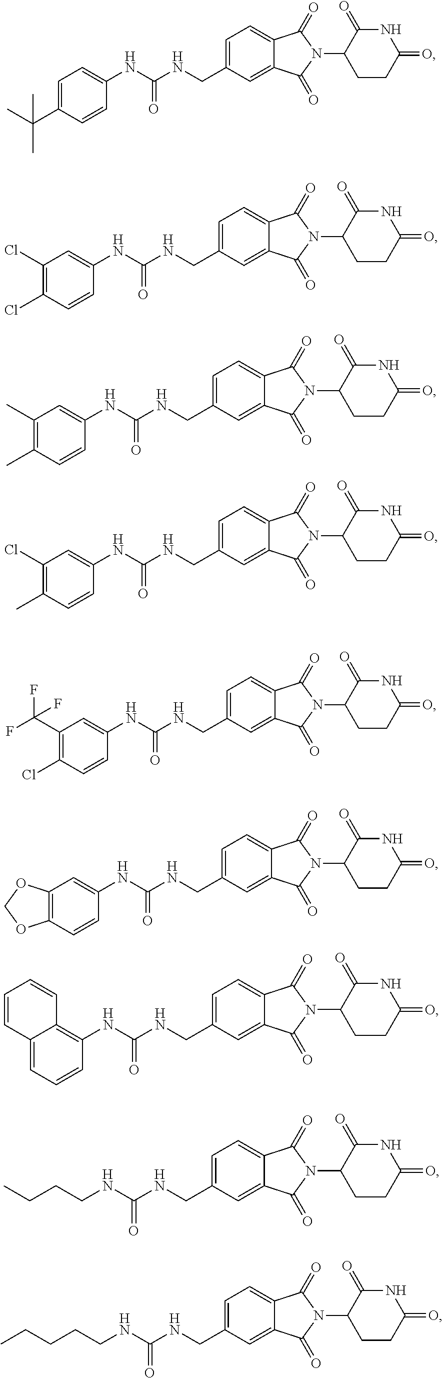

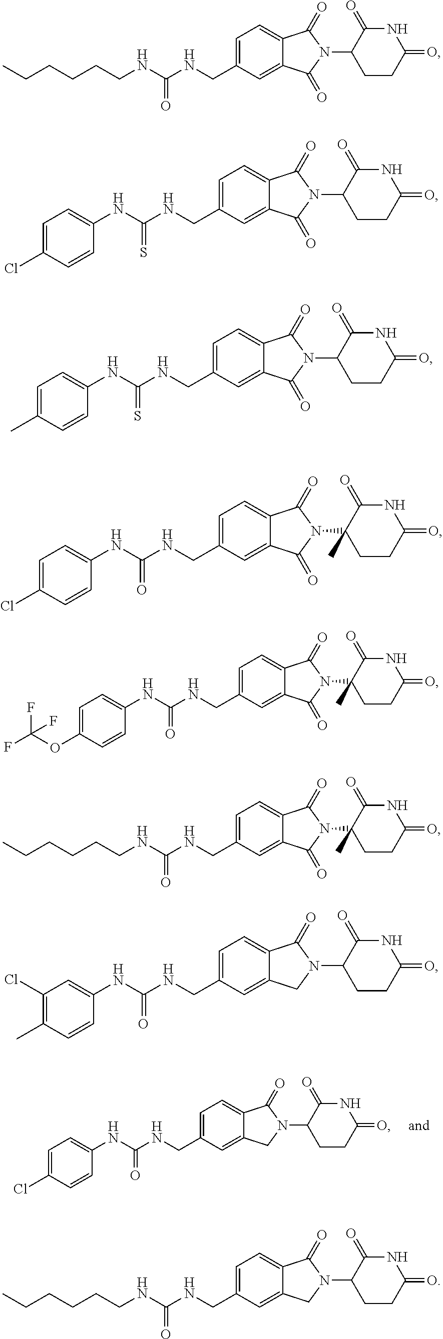

In some embodiments, the treatment compound is selected from a group consisting of:

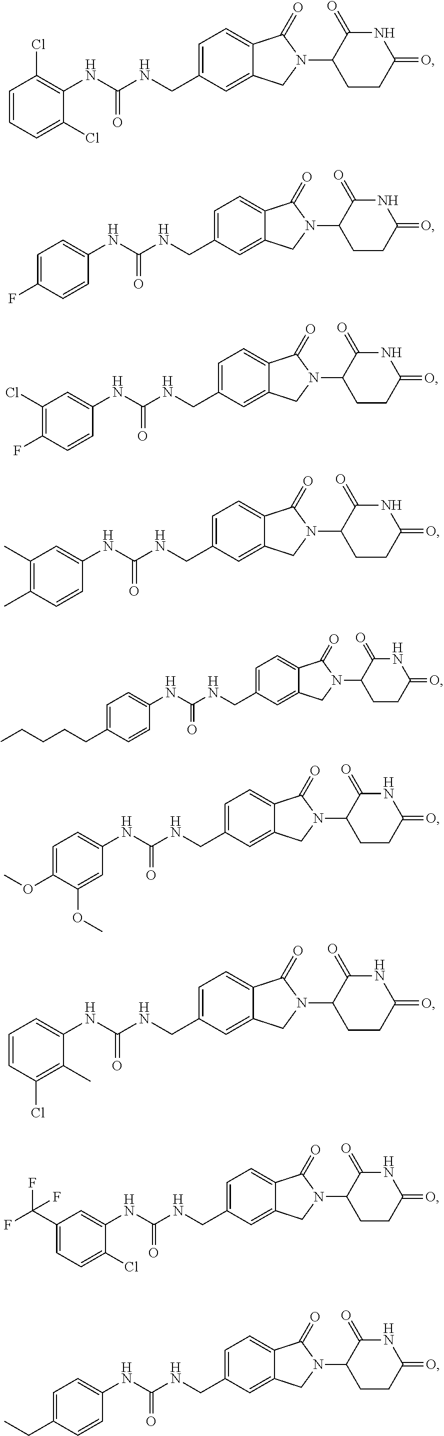

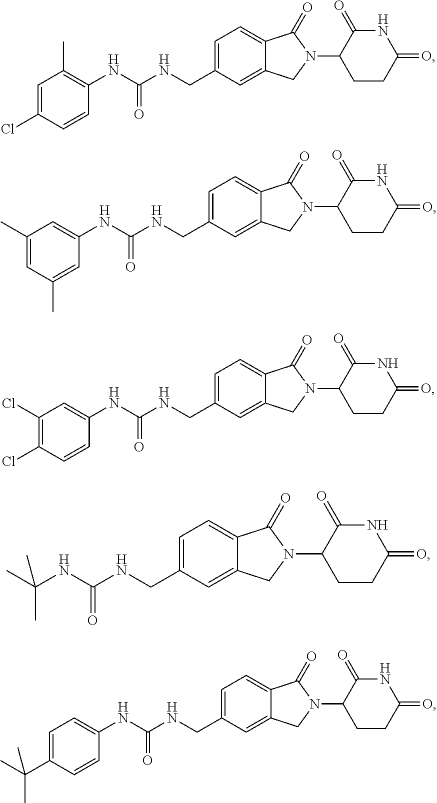 [SMILES: Cc1cc(Cl)ccc1NC(=O)NCc1ccc2c(c1)CN(C1CCC(=O)NC1=O)C2=O ; Cc1cc(C)cc(NC(=O)NCc2ccc3c(c2)CN(C2CCC(=O)NC2=O)C3=O)c1 ; O=C1CCC(N2Cc3cc(CNC(=O)Nc4ccc(Cl)c(Cl)c4)ccc3C2=O)C(=O)N1 ; CC(C)(C)NC(=O)NCc1ccc2c(c1)CN(C1CCC(=O)NC1=O)C2=O ; CC(C)(C)c1ccc(NC(=O)NCc2ccc3c(c2)CN(C2CCC(=O)NC2=O)C3=O)cc1]

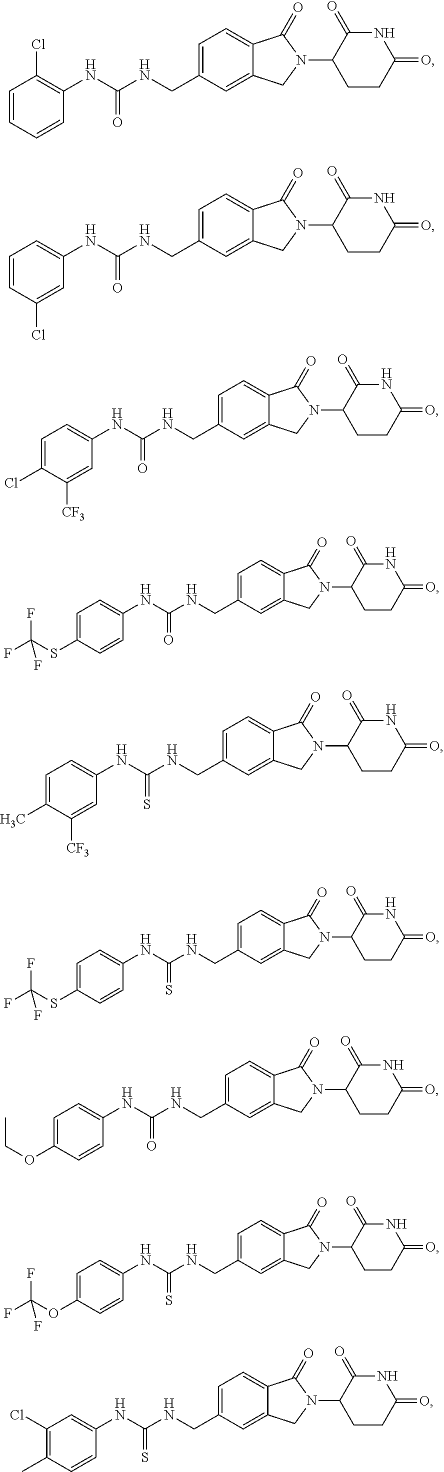

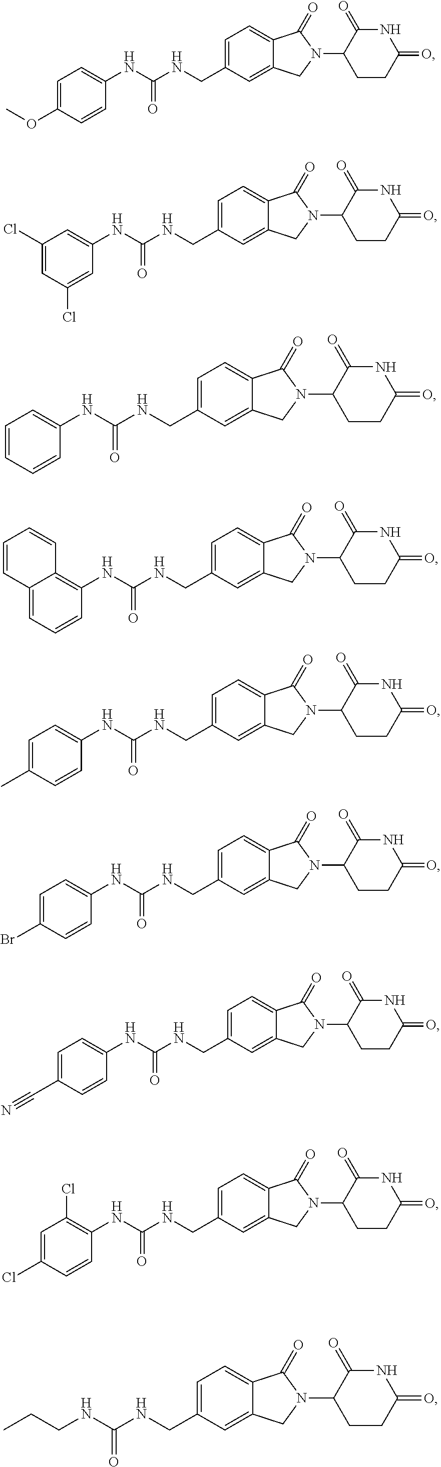

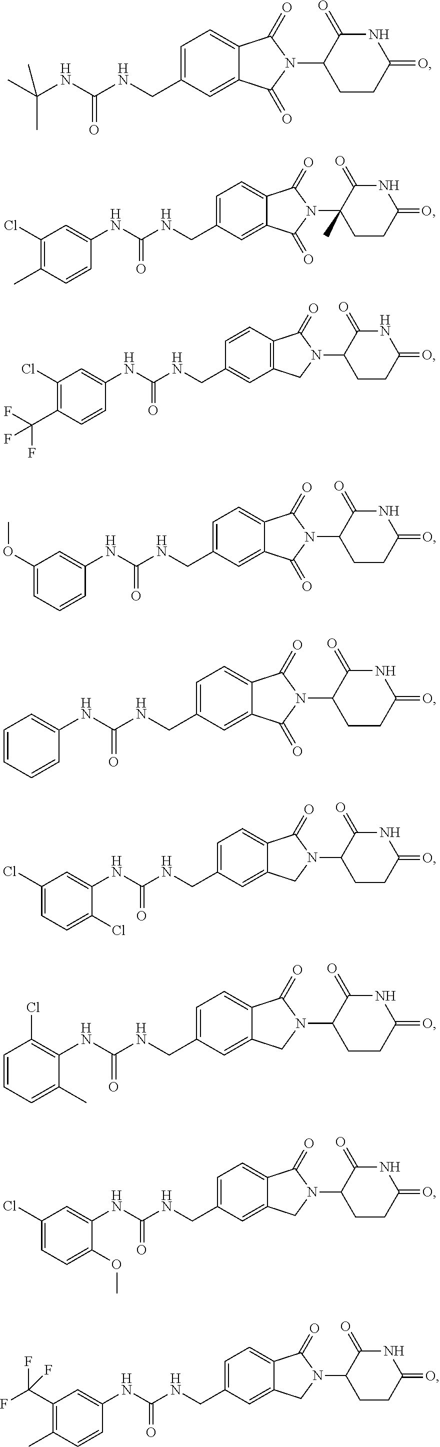

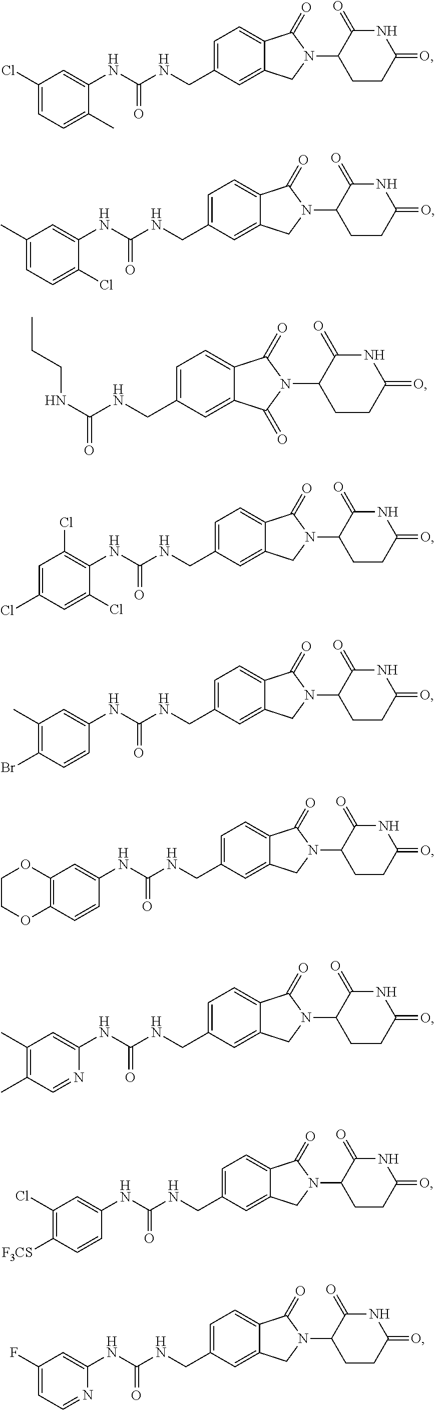

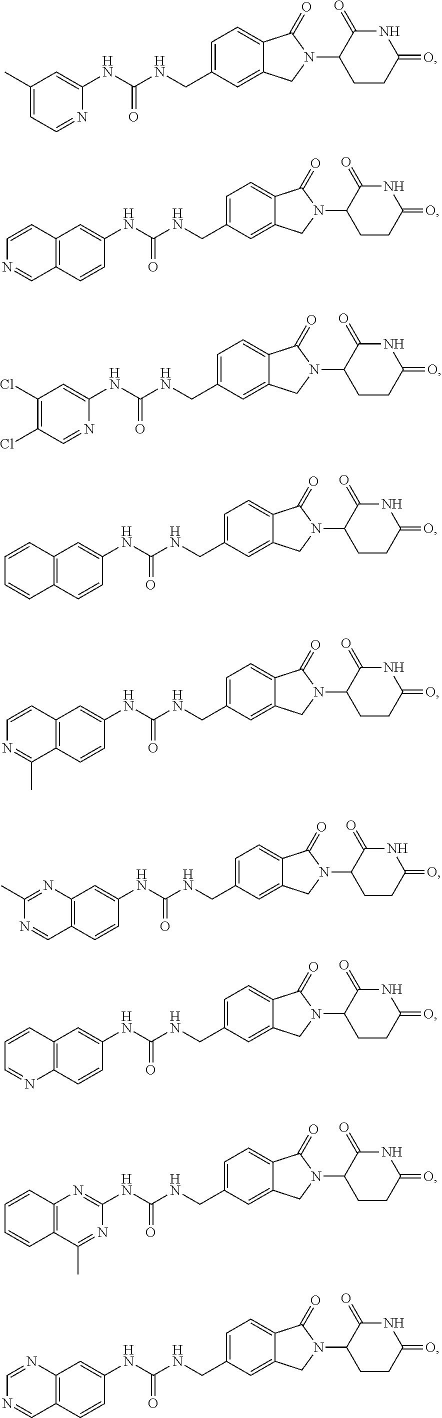

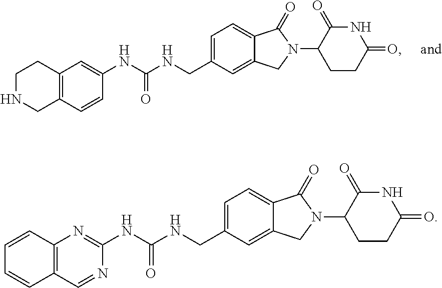 [SMILES: O=C1CCC(N2Cc3cc(CNC(=O)Nc4ccc5c(c4)CCNC5)ccc3C2=O)C(=O)N1 ; "and" ; O=C1CCC(N2Cc3cc(CNC(=O)Nc4ncc5ccccc5n4)ccc3C2=O)C(=O)N1]

In a specific embodiment, the treatment compound is 1-(3-chloro-4-methylphenyl)-3-((2-(2,6-dioxopiperidin-3-yl)-1-oxoisoindolin-5-yl)methyl)urea

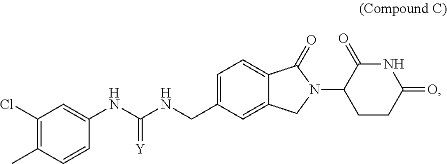

or a pharmaceutically acceptable salt, solvate, stereoisomer, isotopologue, prodrug, hydrate, co-crystal, clathrate, or a polymorph thereof.

In some embodiments, the treatment compound is Compound C, and the cancer is MM, lymphoma, or leukemia. In one embodiment, the cancer is MM. In a specific embodiment, the cancer is lymphoma. In some embodiments, the cancer is leukemia. In another specific embodiment, the leukemia is CLL, CML, ALL, or AML. In one embodiment, the leukemia is AML.

### 5.3. Methods of Detecting and Quantifying Biomarkers

In certain embodiments, provided herein are methods of detecting and quantifying the protein level of biomarker, such as CRBN or a protein that is directly or indirectly affected by CRBN, from a biological sample, comprising contacting proteins within the sample with a first antibody that immunospecifically binds to the biomarker protein. In some embodiments, the methods provided herein further comprise (i) contacting the biomarker protein bound to the first antibody with a second antibody with a detectable label, wherein the second antibody immunospecifically binds to the biomarker protein, and wherein the second antibody immunospecifically binds to a different epitope on the biomarker protein than the first antibody; (ii) detecting the presence of the second antibody bound to the biomarker protein; and (iii) determining the amount of the biomarker protein based on the amount of detectable label in the second antibody. In other embodiments, the methods provided herein further comprise (i) contacting the biomarker protein bound to the first antibody with a second antibody with a detectable label, wherein the second antibody immunospecifically binds to the first antibody; (ii) detecting the presence of the second antibody bound to the first antibody; and (iii) determining the amount of the biomarker protein based on the amount of detectable label in the second antibody.

In some embodiments of the various methods provided herein, the method comprises using dual staining immunohistochemistry to determine the level of a biomarker, such as CRBN or a protein that is directly or indirectly affected by CRBN. In a dual staining immunohistochemistry assay, a biomarker provided herein and another cancer biomarker are simultaneously detected using a first labeled antibody targeting a biomarker provided herein and a second labeled antibody targeting a cancer biomarker. Such assay can improve the specificity, accuracy, and sensitivity for detecting and measuring a biomarker provided herein. In some embodiments, the cancer biomarker is a lymphoma biomarker. In other embodiments, the cancer biomarker is an NHL biomarker. In certain embodiments, the cancer biomarker is a DLBCL biomarker. In some embodiments, the cancer biomarker is an MM biomarker. In other embodiments, the cancer biomarker is a leukemia biomarker. In yet other embodiments, the cancer biomarker is an AML biomarker.

Thus, in some embodiments, the method provided herein comprises (i) contacting proteins within a sample with a first antibody that immunospecifically binds to a biomarker provided herein, the first antibody being coupled with a first detectable label; (ii) contacting the proteins within the sample with a second antibody that immunospecifically binds to a cancer biomarker, the second antibody being coupled with a second detectable label; (iii) detecting the presence of the first antibody and the second antibody bound to the proteins; and (iv) determining the level of the biomarker provided herein based on the amount of detectable label in the first antibody, and determining the level of the cancer biomarker based on the amount of detectable label in the second antibody. In some embodiments, the cancer biomarker is a lymphoma biomarker. In other embodiments, the cancer biomarker is an NHL biomarker. In certain embodiments, the cancer biomarker is a DLBCL biomarker. In some embodiments, the cancer biomarker is an MM biomarker. In other embodiments, the cancer biomarker is a leukemia biomarker. In yet other embodiments, the cancer biomarker is an AML biomarker.

In certain embodiments, provided herein are methods of detecting and quantifying the RNA (e.g., mRNA) level of a biomarker, such as CRBN or a biomarker provided herein, from a biological sample, comprising: (a) obtaining RNA from the sample; (b) contacting the RNA with a primer that specifically binds to a sequence in the RNA to generate a first DNA molecule having a sequence complementary to said RNA; (c) amplifying the DNA corresponding to a segment of a gene encoding the biomarker; and (d) determining the RNA level of the biomarker based on the amount of the amplified DNA.

In some embodiments, the biomarker(s) are evaluated in combination with other biomarker(s) provided herein, such as CRBN, eRF3a, eRF3b, eRF3c, ATF4, ATF3, and DDIT3.

In certain embodiments of the various methods provided herein, the two or more of the steps are performed sequentially. In other embodiments of the methods provided herein, two or more of steps are performed in parallel (e.g., at the same time).

Exemplary assays provided herein for the methods of detecting and quantifying the protein level of a biomarker, such as eRF3a, eRF3b, eRF3c, IKZF1, IKZF3, CK1a, PABP1, eRF1, BIP, eEF1α, PERK, EIF2a, ATF4, ATF3, DDIT3, PPP1R15A, TNFRSF10B, GADD45A, TNFRSF1A, TNFRSF1B, FAS, FADD, IRE1, XBP1, SEC24D, DNAJB9, EDEM1, EDEM2, HYOU1, ATF6, HSPA5, Caspase 3, Caspase 7, Caspase 8, Caspase 9, BID, PARP, Mcl-1 and BAD, or a combination thereof, are immunoassays, such as western blot analysis and enzyme-linked immunosorbent assay (ELISA) (e.g., a sandwich ELISA). An exemplary assay provided herein for the methods of detecting and quantifying the RNA level of a biomarker, such as eRF3a, eRF3b, eRF3c, IKZF1, IKZF3, CK1a, PABP1, eRF1, BIP, eEF1α, PERK, EIF2a, ATF4, ATF3, DDIT3, PPP1R15A, TNFRSF10B, GADD45A, TNFRSF1A, TNFRSF1B, FAS, FADD, IRE1, XBP1, SEC24D, DNAJB9, EDEM1, EDEM2, HYOU1, ATF6, HSPA5, Caspase 3, Caspase 7, Caspase 8, Caspase 9, BID, PARP, Mcl-1 and BAD, or a combination thereof, is reverse transcription polymerase chain reaction (RT-PCR), e.g., quantitative RT-PCR (qRT-PCR).

Exemplary assays provided herein for the methods of detecting and quantifying the protein level of a biomarker, such as CRBN or a protein that is directly or indirectly affected by CRBN (e.g., eRF3a, eRF3b, eRF3c, ATF4, ATF3, and DDIT3), or a combination thereof, are immunoassays, such as western blot analysis and enzyme-linked immunosorbent assay (ELISA) (e.g., a sandwich ELISA). An exemplary assay provided herein for the methods of detecting and quantifying the RNA level of a biomarker, such as CRBN or a protein that is directly or indirectly affected by CRBN (e.g., eRF3a, eRF3b, eRF3c, ATF4, ATF3, and DDIT3), or a combination thereof, is reverse transcription polymerase chain reaction (RT-PCR), e.g., quantitative RT-PCR (qRT-PCR).

### 5.4. Subjects and Samples

In certain embodiments, the various methods provided herein use samples (e.g., biological samples) from subjects or individuals (e.g., patients). The subject can be a patient, such as, a patient with a cancer (e.g., lymphoma, MM, or leukemia). The subject can be a mammal, for example, a human. The subject can be male or female, and can be an adult, a child, or an infant. Samples can be analyzed at a time during an active phase of a cancer (e.g., lymphoma, MM, or leukemia), or when the cancer (e.g., lymphoma, MM, or leukemia) is inactive. In certain embodiments, more than one sample from a subject can be obtained.

In certain embodiments, the sample used in the methods provided herein comprises body fluids from a subject. Non-limiting examples of body fluids include blood (e.g., whole blood), blood plasma, amniotic fluid, aqueous humor, bile, cerumen, cowper's fluid, pre-ejaculatory fluid, chyle, chyme, female ejaculate, interstitial fluid, lymph, menses, breast milk, mucus, pleural fluid, pus, saliva, sebum, semen, serum, sweat, tears, urine, vaginal lubrication, vomit, water, feces, internal body fluids (including cerebrospinal fluid surrounding the brain and the spinal cord), synovial fluid, intracellular fluid (the fluid inside cells), and vitreous humour (the fluid in the eyeball). In some embodiments, the sample is a blood sample. The blood sample can be obtained using conventional techniques as described in, e.g., Innis et al, eds., PCR Protocols (Academic Press, 1990). White blood cells can be separated from blood samples using conventional techniques or commercially available kits, e.g., RosetteSep kit (Stein Cell Technologies, Vancouver, Canada). Sub-populations of white blood cells, e.g., mononuclear cells, B cells, T cells, monocytes, granulocytes, or lymphocytes, can be further isolated using conventional techniques, e.g., magnetically activated cell sorting (MACS) (Miltenyi Biotec, Auburn, Calif.) or fluorescently activated cell sorting (FACS) (Becton Dickinson, San Jose, Calif.).

In one embodiment, the blood sample is from about 0.1 mL to about 10.0 mL, from about 0.2 mL to about 7 mL, from about 0.3 mL to about 5 mL, from about 0.4 mL to about 3.5 mL, or from about 0.5 mL to about 3 mL. In another embodiment, the blood sample is about 0.3, about 0.4, about 0.5, about 0.6, about 0.7, about 0.8, about 0.9, about 1.0, about 1.5, about 2.0, about 2.5, about 3.0, about 3.5, about 4.0, about 4.5, about 5.0, about 6.0, about 7.0, about 8.0, about 9.0, or about 10.0 mL.

In some embodiments, the sample used in the present methods comprises a biopsy (e.g., a tumor biopsy). The biopsy can be from any organ or tissue, for example, skin, liver, lung, heart, colon, kidney, bone marrow, teeth, lymph node, hair, spleen, brain, breast, or other organs. Any biopsy technique known by those skilled in the art can be used for isolating a sample from a subject, for instance, open biopsy, close biopsy, core biopsy, incisional biopsy, excisional biopsy, or fine needle aspiration biopsy.

In one embodiment, the sample used in the methods provided herein is obtained from the subject prior to the subject receiving a treatment for the disease or disorder. In another embodiment, the sample is obtained from the subject during the subject receiving a treatment for the disease or disorder. In another embodiment, the sample is obtained from the subject after the subject receiving a treatment for the disease or disorder. In various embodiments, the treatment comprises administering a compound (e.g., a compound provided in Section 5.7 below) to the subject.

### 5.4. Types of Cells

In certain embodiments, the sample used in the methods provided herein comprises a plurality of cells, such as cancer (e.g., lymphoma, MM, or leukemia) cells. Such cells can include any type of cells, e.g., stem cells, blood cells (e.g., peripheral blood mononuclear cells), lymphocytes, B cells, T cells, monocytes, granulocytes, immune cells, or cancer cells.

B cells (B lymphocytes) include, for example, plasma B cells, memory B cells, B1 cells, B2 cells, marginal-zone B cells, and follicular B cells. B cells can express immunoglobulins (antibodies) and B cell receptor.

Specific cell populations can be obtained using a combination of commercially available antibodies (e.g., antibodies from Quest Diagnostic (San Juan Capistrano, Calif.) or Dako (Denmark)).

In certain embodiments, the cells in the methods provided herein are PBMC. In certain embodiments, the sample used in the methods provided herein is from a disease tissue, e.g., from an individual having cancer (e.g., lymphoma, MM, or leukemia). In certain embodiments, the methods provided herein are useful for detecting gene rearrangement in cells from a healthy individual. In certain embodiments, the number of cells used in the methods provided herein can range from a single cell to about 10⁹ cells. In some embodiments, the number of cells used in the methods provided herein is about 1×10⁴, about 5×10⁴, about 1×10⁵, about 5×10⁵, about 1×10⁶, about 5×10⁶, about 1×10⁷, about 5×10⁷, about 1×10⁸, about 5×10⁸, or about 1×10⁹.

The number and type of cells collected from a subject can be monitored, for example, by measuring changes in cell surface markers using standard cell detection techniques such as flow cytometry, cell sorting, immunocytochemistry (e.g., staining with tissue specific or cell-marker specific antibodies), fluorescence activated cell sorting (FACS), magnetic activated cell sorting (MACS), by examining the morphology of cells using light or confocal microscopy, and/or by measuring changes in gene expression using techniques well known in the art, such as PCR and gene expression profiling. These techniques can be used, too, to identify cells that are positive for one or more particular markers.

In certain embodiments, subsets of cells are used in the methods provided herein. Methods of sorting and isolating specific populations of cells are well-known in the art and can be based on cell size, morphology, or intracellular or extracellular markers. Such methods include, but are not limited to, flow cytometry, flow sorting, FACS, bead based separation such as magnetic cell sorting, size-based separation (e.g., a sieve, an array of obstacles, or a filter), sorting in a microfluidics device, antibody-based separation, sedimentation, affinity adsorption, affinity extraction, density gradient centrifugation, laser capture microdissection, etc. Fluorescence activated cell sorting (FACS) is a well-known method for separating particles, including cells, based on the fluorescent properties of the particles (Kamarch, Methods Enzymol. 1987, 151:150-165). Laser excitation of fluorescent moieties in the individual particles results in a small electrical charge allowing electromagnetic separation of positive and negative particles from a mixture. In one embodiment, cell surface marker-specific antibodies or ligands are labeled with distinct fluorescent labels. Cells are processed through the cell sorter, allowing separation of cells based on their ability to bind to the antibodies used. FACS sorted particles may be directly deposited into individual wells of 96-well or 384-well plates to facilitate separation and cloning.

In one embodiment, RNA (e.g., mRNA) or protein is purified from a tumor, and the presence or absence of a biomarker is measured by gene or protein expression analysis. In certain embodiments, the presence or absence of a biomarker is measured by quantitative real-time PCR (qRT-PCR), microarray, flow cytometry, or immunofluorescence. In other embodiments, the presence or absence of a biomarker is measured by ELISA or other similar methods known in the art.

### 5.5. Methods of Detecting mRNA Levels in a Sample

Several methods of detecting or quantitating mRNA levels are known in the art. Exemplary methods include, but are not limited to, northern blots, ribonuclease protection assays, PCR-based methods, and the like. The mRNA sequence of a biomarker (e.g., the mRNA of CRBN or a protein that is directly or indirectly affected by CRBN, or a fragment thereof) can be used to prepare a probe that is at least partially complementary to the mRNA sequence. The probe can then be used to detect the mRNA in a sample, using any suitable assay, such as PCR-based methods, northern blotting, a dipstick assay, and the like.

In other embodiments, a nucleic acid assay for testing for compound activity in a biological sample can be prepared. An assay typically contains a solid support and at least one nucleic acid contacting the support, where the nucleic acid corresponds to at least a portion of an mRNA that has altered expression during a compound treatment in a patient, such as the mRNA of a biomarker (e.g., CRBN or a protein that is directly or indirectly affected by CRBN). The assay can also have a means for detecting the altered expression of the mRNA in the sample.

The assay method can be varied depending on the type of mRNA information desired. Exemplary methods include but are not limited to Northern blots and PCR-based methods (e.g., qRT-PCR). Methods such as qRT-PCR can also accurately quantitate the amount of the mRNA in a sample.

Any suitable assay platform can be used to determine the presence of mRNA in a sample. For example, an assay may be in the form of a dipstick, a membrane, a chip, a disk, a test strip, a filter, a microsphere, a slide, a multi-well plate, or an optical fiber. An assay system may have a solid support on which a nucleic acid corresponding to the mRNA is attached. The solid support may comprise, for example, a plastic, silicon, a metal, a resin, glass, a membrane, a particle, a precipitate, a gel, a polymer, a sheet, a sphere, a polysaccharide, a capillary, a film, a plate, or a slide. The assay components can be prepared and packaged together as a kit for detecting an mRNA.

The nucleic acid can be labeled, if desired, to make a population of labeled mRNAs. In general, a sample can be labeled using methods that are well known in the art (e.g., using DNA ligase, terminal transferase, or by labeling the RNA backbone, etc.). See, e.g., Ausubel et al., Short Protocols in Molecular Biology (Wiley & Sons, 3rd ed. 1995); Sambrook et al., Molecular Cloning: A Laboratory Manual (Cold Spring Harbor, N.Y., 3rd ed. 2001). In some embodiments, the sample is labeled with fluorescent label. Exemplary fluorescent dyes include, but are not limited to, xanthene dyes, fluorescein dyes (e.g., fluorescein isothiocyanate (FITC), 6-carboxyfluorescein (FAM), 6 carboxy-2′,4′,7′,4,7-hexachlorofluorescein (HEX), 6-carboxy-4′,5′-dichloro-2′,7′-dimethoxyfluorescein (JOE)), rhodamine dyes (e.g., rhodamine 110 (R110), N,N,N′,N′-tetramethyl-6-carboxyrhodamine (TAMRA), 6-carboxy-X-rhodamine (ROX), 5-carboxyrhodamine 6G (R6G5 or G5), 6-carboxyrhodamine 6G (R6G6 or G6)), cyanine dyes (e.g., Cy3, Cy5 and Cy7), Alexa dyes (e.g., Alexa-fluor-555), coumarin, Diethylaminocoumarin, umbelliferone, benzimide dyes (e.g., Hoechst 33258) phenanthridine dyes (e.g., Texas Red), ethidium dyes, acridine dyes, carbazole dyes, phenoxazine dyes, porphyrin dyes, polymethine dyes, BODIPY dyes, quinoline dyes, Pyrene, Fluorescein Chlorotriazinyl, eosin dyes, Tetramethylrhodamine, Lissamine, Napthofluorescein, and the like.

In some embodiments, the mRNA sequences comprise at least one mRNA of a biomarker provided herein. In some embodiments, the biomarker is selected from the group consisting of mRNA of eRF3a, eRF3b, eRF3c, IKZF1, IKZF3, CK1a, PABP1, eRF1, BIP, eEF1α, PERK, EIF2a, ATF4, ATF3, DDIT3, PPP1R15A, TNFRSF10B, GADD45A, TNFRSF1A, TNFRSF1B, FAS, FADD, IRE1, XBP1, SEC24D, DNAJB9, EDEM1, EDEM2, HYOU1, ATF6, HSPA5, Caspase 3, Caspase 7, Caspase 8, Caspase 9, BID, PARP, Mcl-1 and BAD, or a fragment thereof.

In one embodiment, the biomarker is selected from the group consisting of the mRNA of eRF3a, eRF3b, eRF3c, ATF4, ATF3, and DDIT3, or a fragment thereof. In one embodiment, the mRNA is eRF3a mRNA. In another embodiment, the mRNA is eRF3b mRNA. In yet another embodiment, the mRNA is eRF3c mRNA. In another embodiment, the mRNA is ATF4 mRNA. In still another embodiment, the mRNA is ATF3 mRNA. In other embodiments, the mRNA is DDIT3 mRNA. The nucleic acids may be present in specific, addressable locations on a solid support, each corresponding to at least a portion of mRNA sequences that are differentially expressed upon treatment of a compound in a cell or a patient.

A typical mRNA assay method can contain the steps of 1) obtaining surface-bound subject probes; 2) hybridizing a population of mRNAs to the surface-bound probes under conditions sufficient to provide for specific binding; (3) post-hybridization washing to remove nucleic acids not specifically bound to the surface-bound probes; and (4) detecting the hybridized mRNAs. The reagents used in each of these steps and their conditions for use may vary depending on the particular application.

Hybridization can be carried out under suitable hybridization conditions, which may vary in stringency as desired. Typical conditions are sufficient to produce probe/target complexes on a solid surface between complementary binding members, i.e., between surface-bound subject probes and complementary mRNAs in a sample. In certain embodiments, stringent hybridization conditions may be employed.

Hybridization is typically performed under stringent hybridization conditions. Standard hybridization techniques (e.g., under conditions sufficient to provide for specific binding of target mRNAs in the sample to the probes) are described in Kallioniemi et al., Science 1992, 258:818-821 and International Patent Application Publication No. WO 93/18186. Several guides to general techniques are available, e.g., Tijssen, Hybridization with Nucleic Acid Probes, Parts I and II (Elsevier, Amsterdam 1993). For descriptions of techniques suitable for in situ hybridizations, see Gall et al., Meth. Enzymol. 1981, 21:470-480; Angerer et al., Genetic Engineering: Principles and Methods, Vol 7, pgs 43-65 (Plenum Press, New York, Setlow and Hollaender, eds. 1985). Selection of appropriate conditions, including temperature, salt concentration, polynucleotide concentration, hybridization time, stringency of washing conditions, and the like will depend on experimental design, including source of sample, identity of capture agents, degree of complementarity expected, etc., and may be determined as a matter of routine experimentation for those of ordinary skill in the art.

Those of ordinary skill will readily recognize that alternative but comparable hybridization and wash conditions can be utilized to provide conditions of similar stringency.

After the mRNA hybridization procedure, the surface bound polynucleotides are typically washed to remove unbound nucleic acids. Washing may be performed using any convenient washing protocol, where the washing conditions are typically stringent, as described above. The hybridization of the target mRNAs to the probes is then detected using standard techniques.

Other methods, such as PCR-based methods, can also be used to detect the expression of CRBN or a protein that is directly or indirectly affected by CRBN. Examples of PCR methods can be found in U.S. Pat. No. 6,927,024, which is incorporated by reference herein in its entirety. Examples of RT-PCR methods can be found in U.S. Pat. No. 7,122,799, which is incorporated by reference herein in its entirety. A method of fluorescent in situ PCR is described in U.S. Pat. No. 7,186,507, which is incorporated by reference herein in its entirety.

In some embodiments, quantitative Reverse Transcription-PCR (qRT-PCR) can be used for both the detection and quantification of RNA targets (Bustin et al., Clin. Sci. 2005, 109:365-379). Quantitative results obtained by qRT-PCR are generally more informative than qualitative data. Thus, in some embodiments, qRT-PCR-based assays can be useful to measure mRNA levels during cell-based assays. The qRT-PCR method is also useful to monitor patient therapy. Examples of qRT-PCR-based methods can be found, for example, in U.S. Pat. No. 7,101,663, which is incorporated by reference herein in its entirety.

In contrast to regular reverse transcriptase-PCR and analysis by agarose gels, qRT-PCR gives quantitative results. An additional advantage of qRT-PCR is the relative ease and convenience of use. Instruments for qRT-PCR, such as the Applied Biosystems 7500, are available commercially, so are the reagents, such as TagMan® Sequence Detection Chemistry. For example, TagMan® Gene Expression Assays can be used, following the manufacturer's instructions. These kits are pre-formulated gene expression assays for rapid, reliable detection and quantification of human, mouse, and rat mRNA transcripts. An exemplary qRT-PCR program, for example, is 50° C. for 2 minutes, 95° C. for 10 minutes, 40 cycles of 95° C. for 15 seconds, then 60° C. for 1 minute.

To determine the cycle number at which the fluorescence signal associated with a particular amplicon accumulation crosses the threshold (referred to as the C_{T}), the data can be analyzed, for example, using a 7500 Real-Time PCR System Sequence Detection software v1.3 using the comparative C_{T} relative quantification calculation method. Using this method, the output is expressed as a fold-change of expression levels. In some embodiments, the threshold level can be selected to be automatically determined by the software. In some embodiments, the threshold level is set to be above the baseline but sufficiently low to be within the exponential growth region of an amplification curve.

### 5.6. Methods of Detecting Polypeptide or Protein Levels in a Sample

Several protein detection and quantitation methods can be used to measure the level of a biomarker, such as CRBN or a protein that is directly or indirectly affected by CRBN. Any suitable protein quantitation method can be used. In some embodiments, antibody-based methods are used. Exemplary methods that can be used include, but are not limited to, immunoblotting (Western blot), ELISA, immunohistochemistry, flow cytometry, cytometric bead array, mass spectroscopy, and the like. Several types of ELISA are commonly used, including direct ELISA, indirect ELISA, and sandwich ELISA.

In some embodiments, the biomarker is selected from the group consisting of the proteins of eRF3a, eRF3b, eRF3c, IKZF1, IKZF3, CK1a, PABP1, eRF1, BIP, eEF1α, PERK, EIF2a, ATF4, ATF3, DDIT3, PPP1R15A, TNFRSF10B, GADD45A, TNFRSF1A, TNFRSF1B, FAS, FADD, IRE1, XBP1, SEC24D, DNAJB9, EDEM1, EDEM2, HYOU1, ATF6, HSPA5, Caspase 3, Caspase 7, Caspase 8, Caspase 9, BID, PARP, Mcl-1, and BAD. In certain embodiments, the biomarker is a protein that is directly or indirectly affected by CRBN. In one embodiment, the biomarker is selected from a group consisting of eRF3a, eRF3b, eRF3c, ATF4, ATF3, and DDIT3. In some embodiments, the biomarker is selected from a group consisting of eRF3a, eRF3b, and eRF3c. In other embodiments, the biomarker is selected from a group consisting of ATF4, ATF3, and DDIT3. In a specific embodiment, the biomarker is eRF3a. In another specific embodiment, the biomarker is eRF3b. In yet another specific embodiment, the biomarker is eRF3c. In another embodiment, the biomarker is ATF4. In still another specific embodiment, the biomarker is ATF3. In yet another specific embodiment, the biomarker is DDIT3.

### 5.7. Compounds

Various compounds provided herein contain one or more chiral centers, and can exist as mixtures of enantiomers (e.g., racemic mixtures) or mixtures of diastereomers. The methods provided herein encompass the use of stereomerically pure forms of such compounds as well as mixtures of those forms. For example, mixtures comprising equal or unequal amounts of the enantiomers of a particular compound may be used in methods provided herein. These isomers may be asymmetrically synthesized or resolved using standard techniques, such as chiral columns or chiral resolving agents. See, Jacques et al., Enantiomers, Racemates and Resolutions (Wiley-Interscience, New York, 1981); Wilen et al., Tetrahedron 1977, 33:2725-2736; Eliel, Stereochemistry of Carbon Compounds (McGraw-Hill, N Y, 1962); Wilen, Tables of Resolving Agents and Optical Resolutions, p. 268 (Eliel, ed., Univ. of Notre Dame Press, Notre Dame, Ind., 1972).

In some embodiments, this invention encompasses compounds of formula (I):

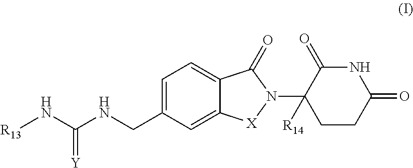

or a pharmaceutically acceptable salt, solvate, stereoisomer, isotopologue, prodrug, hydrate, co-crystal, clathrate, or a polymorph thereof, wherein:

### X is CH₂ or C═O;

### Y is O or S;

### R¹³ is:

(C₁-C₁₀)alkyl; (C₁-C₁₀)alkoxy;
5 to 10 membered aryl or heteroaryl, optionally substituted with one or more of: halogen; cyano; (C₁-C₆)alkylenedioxy; (C₁-C₆)alkoxy, itself optionally substituted with one or more halogen; (C₁-C₆)alkyl, itself optionally substituted with one or more halogen; or (C₁-C₆)alkylthio, itself optionally substituted with one or more halogen; and
   R¹⁴ is H or (C₁-C₆)alkyl.

In one embodiment, X is CH₂. In another embodiment, X is C═O.

In one embodiment, Y is O. In another embodiment, Y is S.

In one embodiment, R¹³ is (C₁-C₁₀)alkyl. In certain specific embodiments, R¹³ is (C₁-C₆)alkyl. In certain specific embodiments, R¹³ is propyl, butyl, pentyl, or hexyl.

In one embodiment, R¹³ is (C₁-C₁₀)alkoxy.

In one embodiment, R¹³ is 5 to 10 membered aryl or heteroaryl, optionally substituted with cyano. In certain specific embodiments, R13 is phenyl, optionally substituted with cyano.

In one embodiment, R¹³ is 5 to 10 membered aryl or heteroaryl, optionally substituted with (C₁-C₆)alkylenedioxy. In certain specific embodiments, R¹³ is phenyl, optionally substituted with methylenedioxy.

In one embodiment, R¹³ is 5 to 10 membered aryl or heteroaryl, optionally substituted with one or more halogen. In certain specific embodiments, R¹³ is phenyl, optionally substituted with one or more halogen.

In another embodiment, R¹³ is 5 to 10 membered aryl or heteroaryl, optionally substituted with (C₁-C₆)alkyl or (C₁-C₆)alkoxy, themselves optionally substituted with one or more halogens. In certain specific embodiments, R¹³ is phenyl, optionally substituted with methyl or methoxy, themselves optionally substituted with 1, 2, or 3 halogens.

In another embodiment, R¹³ is 5 to 10 membered aryl or heteroaryl, optionally substituted with (C₁-C₆)alkylthio, itself optionally substituted with one or more halogens.

In another embodiment, R¹⁴ is H. In another embodiment, R¹⁴ is (C₁-C₆)alkyl. In certain specific embodiments, R¹⁴ is methyl.

All of the combinations of the above embodiments are encompassed by this invention.

Examples include, but are not limited to, those listed below, or a pharmaceutically acceptable salt, solvate, stereoisomer, isotopologue, prodrug, hydrate, co-crystal, clathrate, or a polymorph thereof:

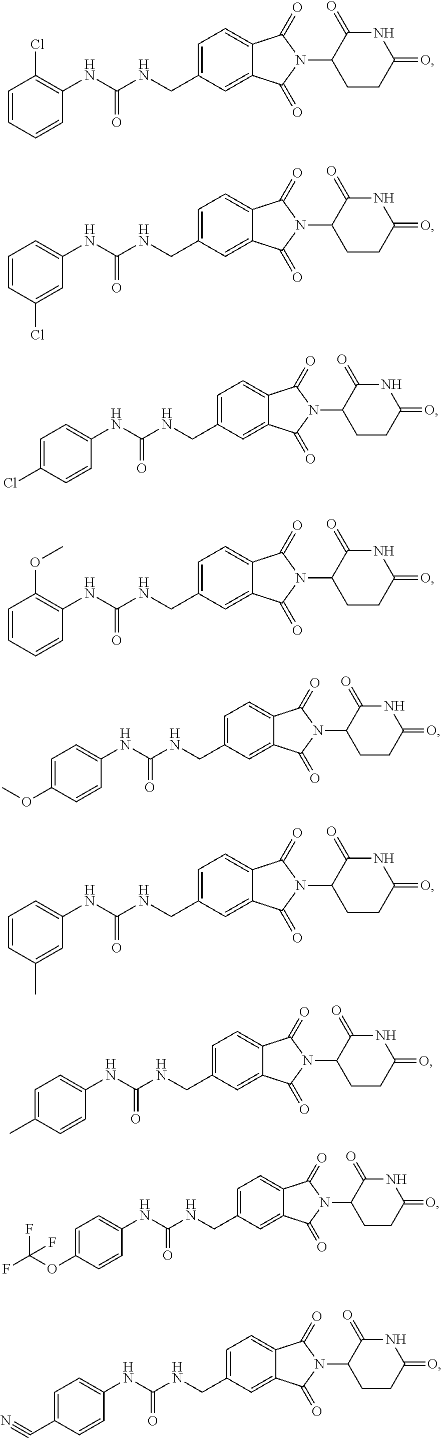

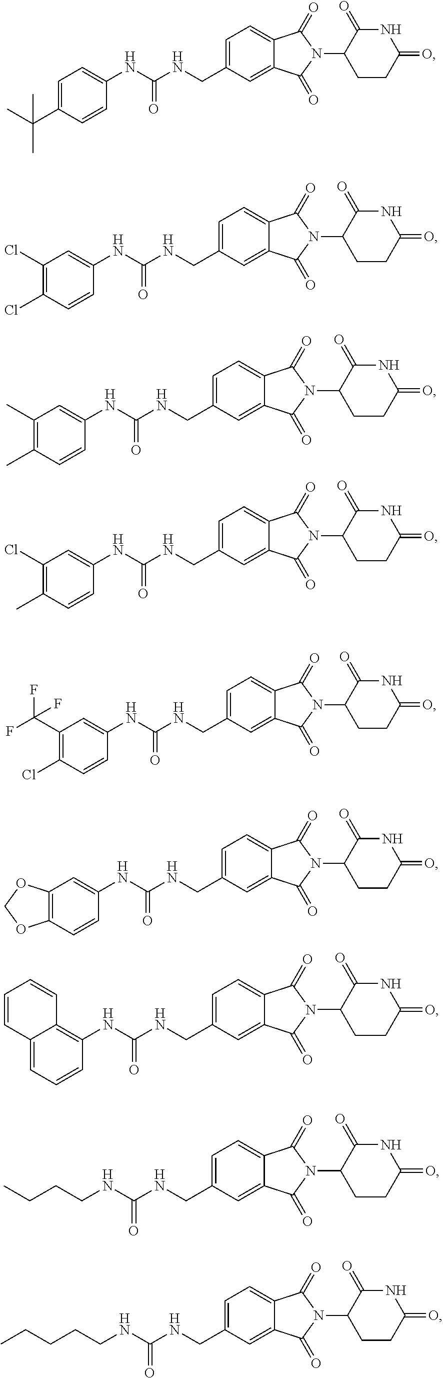

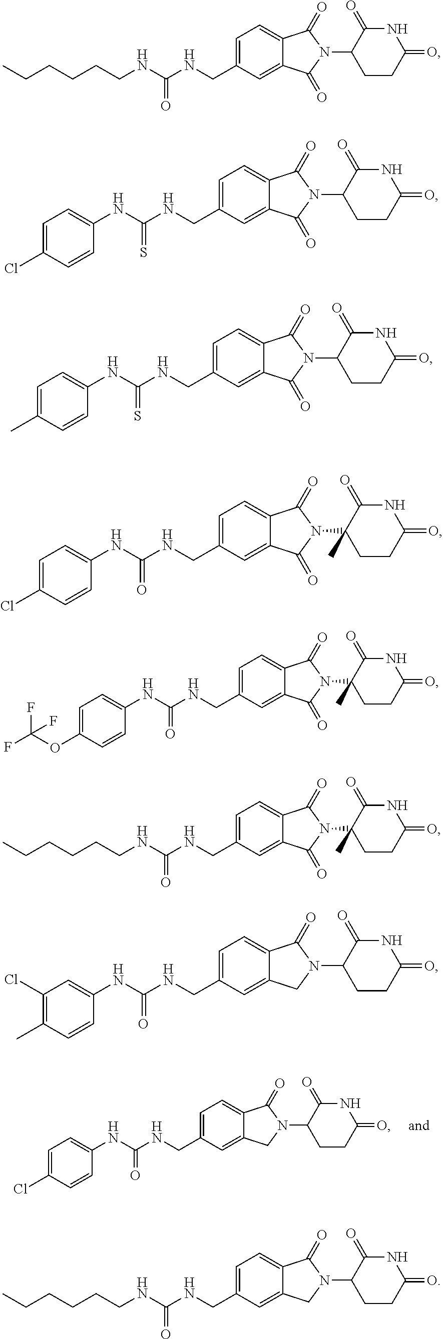

Other examples include, but are not limited to, those listed below, or a pharmaceutically acceptable salt, solvate, stereoisomer, isotopologue, prodrug, hydrate, co-crystal, clathrate, or a polymorph thereof:

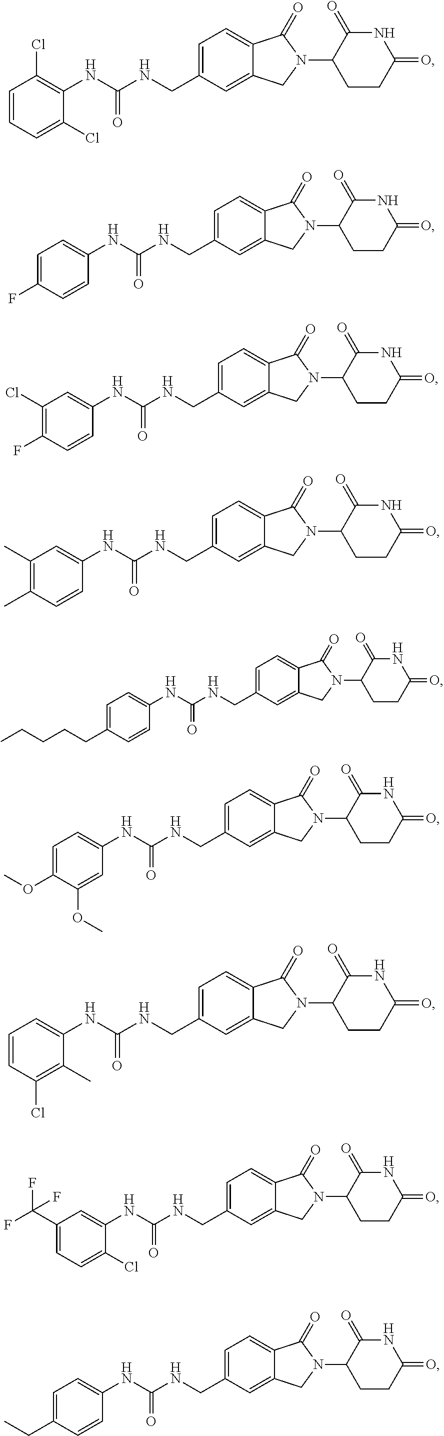

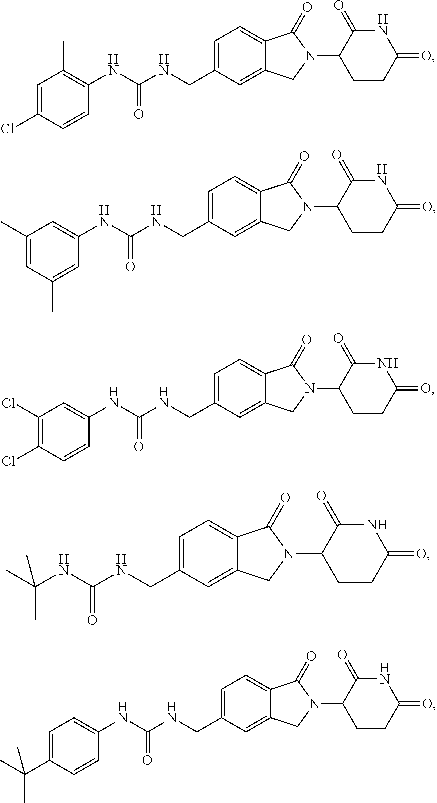 [SMILES: Cc1cc(Cl)ccc1NC(=O)NCc1ccc2c(c1)CN(C1CCC(=O)NC1=O)C2=O ; Cc1cc(C)cc(NC(=O)NCc2ccc3c(c2)CN(C2CCC(=O)NC2=O)C3=O)c1 ; O=C1CCC(N2Cc3cc(CNC(=O)Nc4ccc(Cl)c(Cl)c4)ccc3C2=O)C(=O)N1 ; CC(C)(C)NC(=O)NCc1ccc2c(c1)CN(C1CCC(=O)NC1=O)C2=O ; CC(C)(C)c1ccc(NC(=O)NCc2ccc3c(c2)CN(C2CCC(=O)NC2=O)C3=O)cc1]

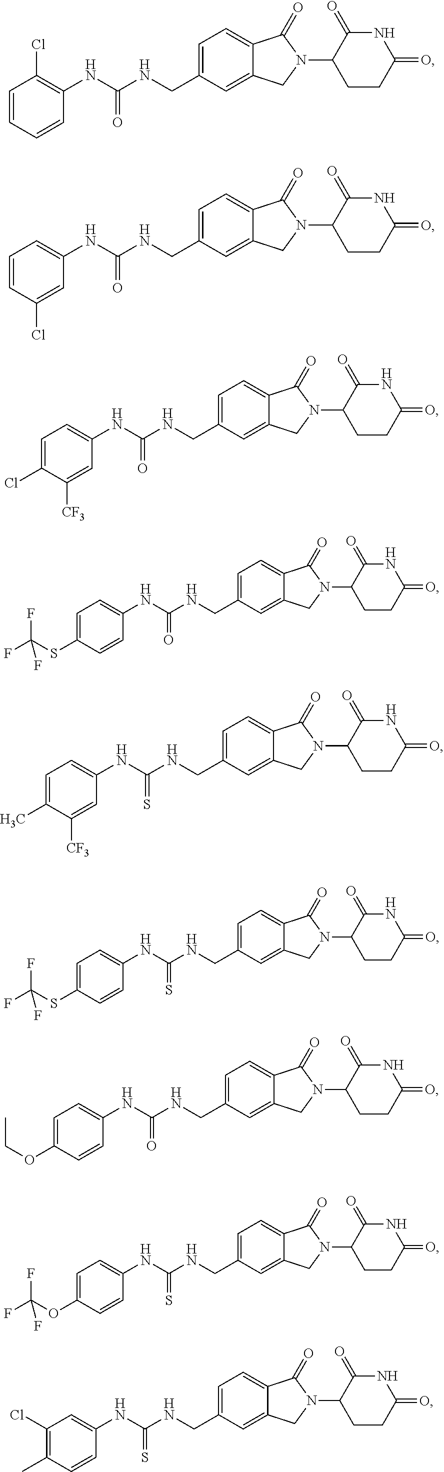

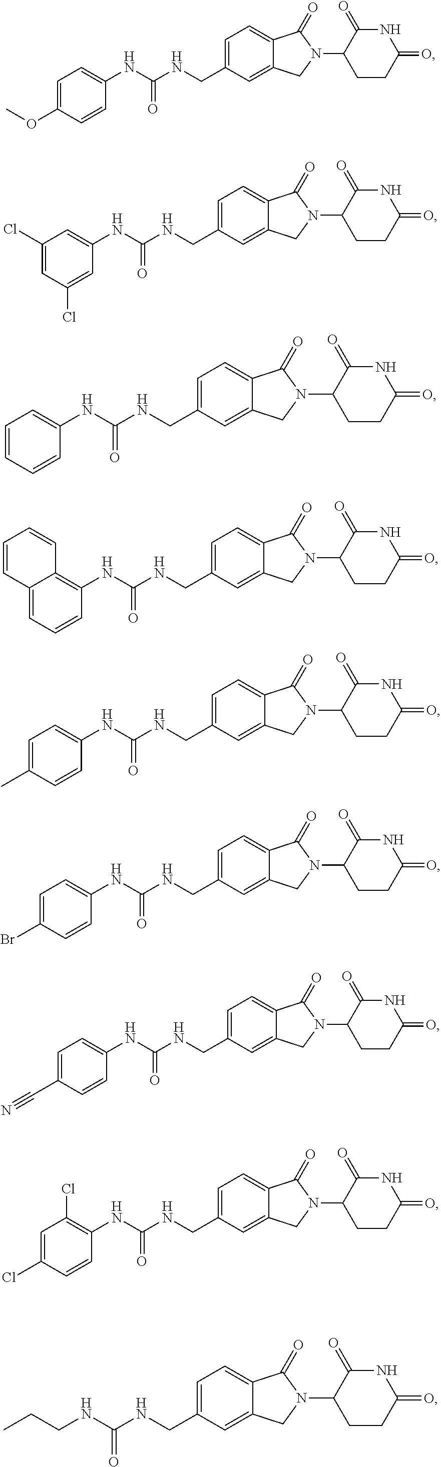

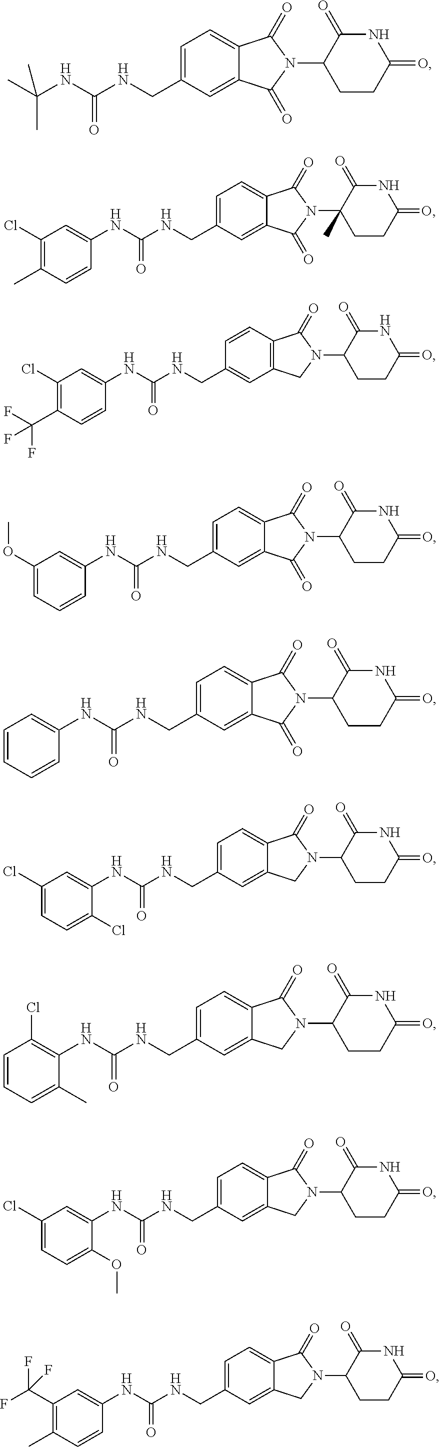

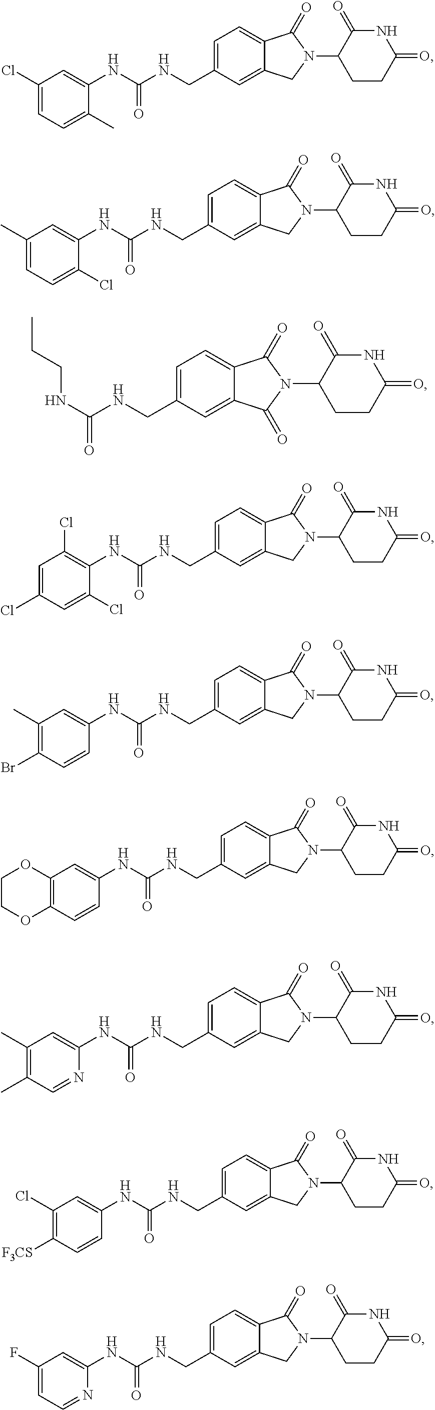

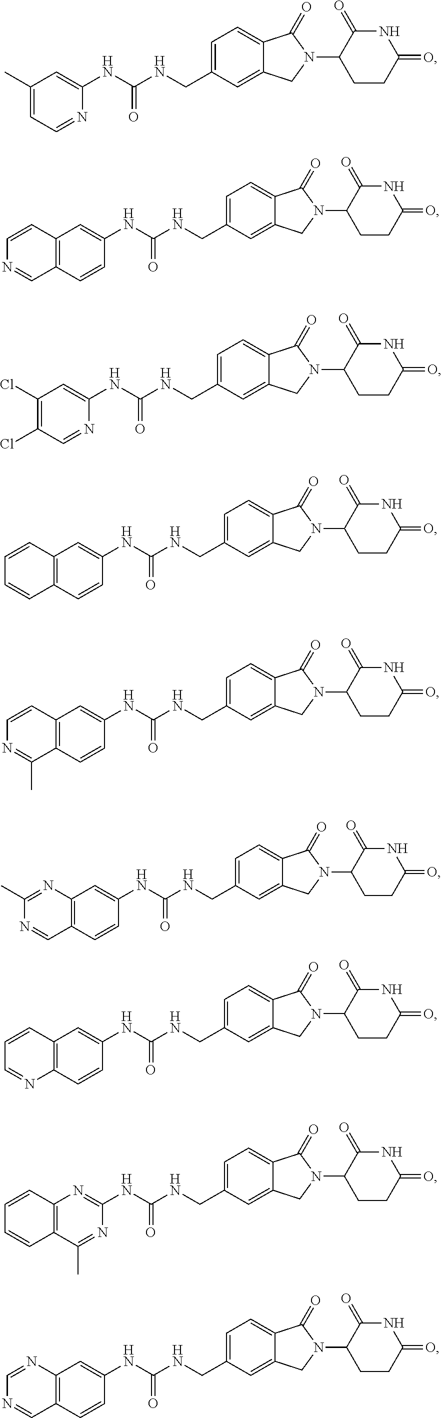

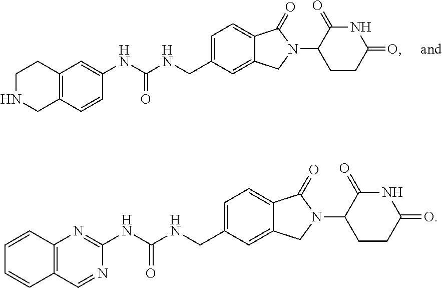 [SMILES: O=C1CCC(N2Cc3cc(CNC(=O)Nc4ccc5c(c4)CCNC5)ccc3C2=O)C(=O)N1 ; "and" ; O=C1CCC(N2Cc3cc(CNC(=O)Nc4ncc5ccccc5n4)ccc3C2=O)C(=O)N1]

In a specific embodiment, the treatment compound is 1-(3-chloro-4-methylphenyl)-3-((2-(2,6-dioxopiperidin-3-yl)-1-oxoisoindolin-5-yl)methyl)urea

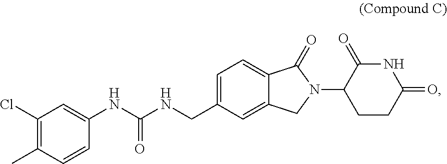 [SMILES: Cc1ccc(NC(=O)NCc2ccc3c(c2)CN(C2CCC(=O)NC2=O)C3=O)cc1Cl]

or a pharmaceutically acceptable salt, solvate, stereoisomer, isotopologue, prodrug, hydrate, co-crystal, clathrate, or a polymorph thereof.

It is understood that the foregoing detailed description and accompanying examples are merely illustrative, and are not to be taken as limitations upon the scope of the subject matter. Various changes and modifications to the disclosed embodiments will be apparent to those skilled in the art. Such changes and modifications, including without limitation those relating to the chemical structures, substituents, derivatives, intermediates, syntheses, formulations, and/or methods of use provided herein, may be made without departing from the spirit and scope thereof. U.S. patents and publications referenced herein are incorporated by reference.

### 5.8 Pharmaceutical Compositions

In certain embodiments, provided herein are pharmaceutical compositions comprising a compound of Formula I, or a pharmaceutically acceptable salt, solvate, stereoisomer, isotopologue, prodrug, hydrate, co-crystal, clathrate, or a polymorph thereof. In some embodiments, the pharmaceutical compositions provided herein contain therapeutically effective amounts of one or more of the compounds provided herein and a pharmaceutically acceptable carrier, diluents, or excipient. In some embodiments, the compounds may be formulated as the sole pharmaceutically active ingredient in the composition or may be combined with other active ingredients. In certain embodiments, the compound of Formula I is Compound C.

The compounds can be formulated into suitable pharmaceutical compositions for different routes of administration, such as oral, injection, sublingual and buccal, rectal, vaginal, ocular, otic, nasal, inhalation, nebulization, cutaneous, or transdermal. Typically the compounds described above are formulated into pharmaceutical compositions using techniques and procedures well known in the art (see, e.g., Ansel, Introduction to Pharmaceutical Dosage Forms, (7th ed. 1999)).

In the compositions, effective concentrations of one or more compounds or pharmaceutically acceptable salts are mixed with a suitable pharmaceutical carrier or vehicle. In certain embodiments, the concentrations of the compounds in the compositions are effective for delivery of an amount, upon administration, that treats, prevents, or ameliorates one or more of the symptoms and/or progression of cancer, including solid cancer and blood borne cancer.

The active compound is in an amount sufficient to exert a therapeutically useful effect in the absence of undesirable side effects on the patient treated. The therapeutically effective concentration may be determined empirically by testing the compounds in in vitro and in vivo systems described herein and then extrapolated therefrom for dosages for humans. The concentration of active compound in the pharmaceutical composition will depend on absorption, tissue distribution, inactivation, and excretion rates of the active compound, the physicochemical characteristics of the compound, the dosage schedule, and amount administered as well as other factors known to those of skill in the art.

The pharmaceutically therapeutically active compounds and salts thereof are formulated and administered in unit dosage forms or multiple dosage forms. Unit dose forms as used herein refer to physically discrete units suitable for human and animal subjects and packaged individually as is known in the art. Each unit dose contains a predetermined quantity of the therapeutically active compound sufficient to produce the desired therapeutic effect, in association with the required pharmaceutical carriers, vehicles, or diluents. Examples of unit dose forms include ampoules and syringes and individually packaged tablets or capsules. Unit dose forms may be administered in fractions or multiples thereof. A multiple dose form is a plurality of identical unit dosage forms packaged in a single container to be administered in segregated unit dose form. Examples of multiple dose forms include vials, bottles of tablets or capsules, or bottles of pints or gallons. Hence, multiple dose form is a multiple of unit doses which are not segregated in packaging.

It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

For oral administration, a pharmaceutically acceptable non-toxic composition is formed by the incorporation of any of the normally employed excipients, for example, pharmaceutical grades of mannitol, lactose, starch, magnesium stearate, talcum, cellulose derivatives, sodium crosscarmellose, glucose, sucrose, magnesium carbonate, or sodium saccharin. Such compositions include solutions, suspensions, tablets, capsules, powders, sustained release formulations (such as, but not limited to, implants and microencapsulated delivery systems), and biodegradable, biocompatible polymers (such as collagen, ethylene vinyl acetate, polyanhydrides, polyglycolic acid, polyorthoesters, polylactic acid, and others). Methods for preparation of these compositions are known to those skilled in the art.

Solutions or suspensions used for parenteral, intradermal, subcutaneous, or topical application can include any of the following components: a sterile diluents (such as water, saline solution, fixed oil, polyethylene glycol, glycerine, propylene glycol, dimethyl acetamide, or other synthetic solvent), antimicrobial agents (such as benzyl alcohol and methyl parabens), antioxidants (such as ascorbic acid and sodium bisulfate), chelating agents (such as ethylenediaminetetraacetic acid (EDTA)), buffers (such as acetates, citrates, and phosphates), and agents for the adjustment of tonicity (such as sodium chloride or dextrose). Parenteral preparations can be enclosed in ampoules, pens, disposable syringes, or single or multiple dose vials made of glass, plastic, or other suitable material.

In instances in which the compounds exhibit insufficient solubility, methods for solubilizing compounds may be used. Such methods are known to those of skill in this art, and include, but are not limited to, using cosolvents, such as dimethylsulfoxide (DMSO), using surfactants, such as TWEEN®, or dissolving the compound in aqueous sodium hydroxide, sodium bicarbonate, or hydrochloric acid.

Sustained-release preparations can also be prepared. Suitable examples of sustained-release preparations include semipermeable matrices of solid hydrophobic polymers containing the compound provided herein, which matrices are in the form of shaped articles, e.g., films or microcapsule. Examples of sustained-release matrices include iontophoresis patches, polyesters, hydrogels (for example, poly(2-hydroxyethyl-methacrylate) or poly(vinylalcohol)), polylactides, copolymers of L-glutamic acid and ethyl-L-glutamate, non-degradable ethylene-vinyl acetate, degradable lactic acid-glycolic acid copolymers such as LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated compound remain in the body for a long time, they may denature or aggregate as a result of exposure to moisture at 37° C., resulting in a loss of biological activity and possible changes in their structure. Rational strategies can be devised for stabilization depending on the mechanism of action involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

Lactose-free compositions provided herein can contain excipients that are well known in the art and are listed, for example, in The U.S. Pharmacopeia (USP). In general, lactose-free compositions contain an active ingredient, a binder/filler, and a lubricant in pharmaceutically compatible and pharmaceutically acceptable amounts. Exemplary lactose-free dosage forms contain an active ingredient, microcrystalline cellulose, pre-gelatinized starch, and magnesium stearate.

Further encompassed are anhydrous pharmaceutical compositions and dosage forms containing a compound provided herein. Anhydrous pharmaceutical compositions and dosage forms provided herein can be prepared using anhydrous or low moisture containing ingredients and low moisture or low humidity conditions, as known by those skilled in the art. An anhydrous pharmaceutical composition should be prepared and stored such that its anhydrous nature is maintained. Accordingly, anhydrous compositions are packaged using materials known to prevent exposure to water such that they can be included in suitable formulatory kits. Examples of suitable packaging include, but are not limited to, hermetically sealed foils, plastics, unit dose containers (e.g., vials), blister packs, and strip packs.

Dosage forms or compositions containing active ingredient in the range of 0.001% to 100% with the balance made up from non-toxic carrier may be prepared. In some embodiments, the contemplated compositions contain from about 0.005% to about 95% active ingredient. In other embodiments, the contemplated compositions contain from about 0.01% to about 90% active ingredient. In certain embodiments, the contemplated compositions contain from about 0.1% to about 85% active ingredient. In other embodiments, the contemplated compositions contain from about 0.1% to about 75-95% active ingredient.

The compositions may include other active compounds to obtain desired combinations of properties. The compounds provided herein, or pharmaceutically acceptable salts thereof as described herein, may also be advantageously administered for therapeutic or prophylactic purposes together with another pharmacological agent known in the general art to be of value in treating one or more of the diseases or medical conditions referred to herein above, such as solid cancer or blood born cancer. It is to be understood that such combination therapy constitutes a further aspect of the compositions and methods of treatment provided herein.

5.8.1 Oral Dosage Forms

Oral pharmaceutical dosage forms are either solid, gel, or liquid. The solid dosage forms are tablets, capsules, granules, and bulk powders. Types of oral tablets include compressed, chewable lozenges, and tablets, which may be enteric coated, sugar coated, or film coated. Capsules may be hard or soft gelatin capsules, while granules and powders may be provided in non-effervescent or effervescent form with the combination of other ingredients known to those skilled in the art.

In certain embodiments, the formulations are solid dosage forms, such as capsules or tablets. The tablets, pills, capsules, troches, and the like, can contain any one or combination of the following ingredients, or compounds of a similar nature: a binder, a diluents, a lubricant, a glidant, a disintegrating agent, a coloring agent, a sweetening agent, a flavoring agent, a wetting agent, and a coating (e.g., an enteric coating or a film coating).

Examples of binders include microcrystalline cellulose, gum tragacanth, glucose solution, acacia mucilage, gelatin solution, sucrose, and starch paste. Diluents include, for example, lactose, sucrose, starch, kaolin, salt, mannitol, and dicalcium phosphate. Lubricants include, for example, talc, starch, magnesium or calcium stearate, lycopodium, and stearic acid. Glidants include, but are not limited to, colloidal silicon dioxide. Disintegrating agents include, for example, crosscarmellose sodium, sodium starch glycolate, alginic acid, corn starch, potato starch, bentonite, methylcellulose, agar, and carboxymethylcellulose. Coloring agents include, for example, any of the approved certified water soluble FD and C dyes, mixtures thereof, and water insoluble FD and C dyes suspended on alumina hydrate. Sweetening agents include, for example, sucrose, lactose, mannitol, artificial sweetening agents such as saccharin, and any number of spray dried flavors. Flavoring agents include, for example, natural flavors extracted from plants such as fruits, and synthetic blends of compounds, which produce a pleasant sensation, including but not limited to peppermint and methyl salicylate. Wetting agents include, for example, propylene glycol monostearate, sorbitan monooleate, diethylene glycol monolaurate, and polyoxyethylene laural ether. Enteric coatings include, for example, fatty acids, fats, waxes, shellac, ammoniated shellac, and cellulose acetate phthalates. Film coatings include, for example, hydroxyethylcellulose, sodium carboxymethylcellulose, polyethylene glycol 4000, and cellulose acetate phthalate.

If oral administration is desired, the compound could be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

Liquid oral dosage forms include aqueous solutions, emulsions, suspensions, solutions, and/or suspensions reconstituted from non-effervescent granules and effervescent preparations reconstituted from effervescent granules. Aqueous solutions include, for example, elixirs and syrups. Emulsions are either oil-in-water or water-in-oil. Elixirs are clear, sweetened, hydroalcoholic preparations. Pharmaceutically acceptable carriers used in elixirs include solvents. Syrups are concentrated aqueous solutions of a sugar, for example, sucrose, and may contain a preservative. An emulsion is a two phase system in which one liquid is dispersed in the form of small globules throughout another liquid. Pharmaceutically acceptable carriers used in emulsions are non aqueous liquids, emulsifying agents, and preservatives. Suspensions use pharmaceutically acceptable suspending agents and preservatives. Pharmaceutically acceptable substances used in non-effervescent granules, to be reconstituted into a liquid oral dosage form, include diluents, sweeteners, and wetting agents. Pharmaceutically acceptable substances used in effervescent granules, to be reconstituted into a liquid oral dosage form, include organic acids and a source of carbon dioxide. Coloring and flavoring agents are used in all of the above dosage forms.

Solvents include glycerin, sorbitol, ethyl alcohol, and syrup. Examples of preservatives include glycerin, methyl and propylparaben, benzoic add, sodium benzoate, and alcohol. Examples of non aqueous liquids utilized in emulsions include mineral oil and cottonseed oil. Examples of emulsifying agents include gelatin, acacia, tragacanth, bentonite, and surfactants such as polyoxyethylene sorbitan monooleate. Suspending agents include sodium carboxymethylcellulose, pectin, tragacanth, Veegum, and acacia. Diluents include lactose and sucrose. Sweetening agents include sucrose, syrups, glycerin, and artificial sweetening agents such as saccharin. Wetting agents include propylene glycol monostearate, sorbitan monooleate, diethylene glycol monolaurate, and polyoxyethylene lauryl ether. Organic acids include citric and tartaric acid. Sources of carbon dioxide include sodium bicarbonate and sodium carbonate.

For a solid dosage form, the solution or suspension in, for example, propylene carbonate, vegetable oils, or triglycerides, is encapsulated in a gelatin capsule. Such solutions, and the preparation and encapsulation thereof, are disclosed in U.S. Pat. Nos. 4,328,245; 4,409,239; and 4,410,545. For a liquid dosage form, the solution, for example, in a polyethylene glycol, may be diluted with a sufficient quantity of a pharmaceutically acceptable liquid carrier, e.g., water, to be easily measured for administration.

Alternatively, liquid or semi solid oral formulations may be prepared by dissolving or dispersing the active compound or salt in vegetable oils, glycols, triglycerides, propylene glycol esters (e.g., propylene carbonate), and other such carriers, and encapsulating these solutions or suspensions in hard or soft gelatin capsule shells. Other useful formulations include, but are not limited to, those containing a compound provided herein, a dialkylated mono- or poly-alkylene glycol, including but not limited to, 1,2-dimethoxymethane, diglyme, triglyme, tetraglyme, polyethylene glycol-350-dimethyl ether, polyethylene glycol-550-dimethyl ether, polyethylene glycol-750-dimethyl ether, wherein 350, 550, and 750 refer to the approximate average molecular weight of the polyethylene glycol, and one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, thiodipropionic acid and its esters, and dithiocarbamates.

Other formulations include, but are not limited to, aqueous alcoholic solutions including a pharmaceutically acceptable acetal. Alcohols used in these formulations are any pharmaceutically acceptable water-miscible solvents having one or more hydroxyl groups, including, but not limited to, propylene glycol and ethanol. Acetals include, but are not limited to, di(lower alkyl) acetals of lower alkyl aldehydes such as acetaldehyde diethyl acetal.

5.8.2 Injectables, Solutions, and Emulsions

Parenteral administration of the compositions includes intravenous, subcutaneous, and intramuscular administrations. Compositions for parenteral administration include sterile solutions ready for injection, sterile dry soluble products, such as lyophilized powders, ready to be combined with a solvent just prior to use, sterile suspensions ready for injection, and sterile emulsions. The solutions may be either aqueous or nonaqueous. The unit dose parenteral preparations are packaged in an ampoule, a vial or a syringe with a needle. All preparations for parenteral administration must be sterile, as is known and practiced in the art.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents, and other pharmaceutically acceptable substances.

Examples of aqueous vehicles include sodium chloride injection, Ringer's injection, isotonic dextrose injection, sterile water injection, dextrose and lactated Ringer's injection. Nonaqueous parenteral vehicles include fixed oils of vegetable origin, such as cottonseed oil, corn oil, sesame oil, and peanut oil. Antimicrobial agents in bacteriostatic or fungistatic concentrations must be added to parenteral preparations packaged in multiple dose containers, which include phenols or cresols, mercurials, benzyl alcohol, chlorobutanol, methyl and propyl-p-hydroxybenzoic acid esters, thimerosal, benzalkonium chloride, and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN® 80). A sequestering or chelating agent of metal ions includes EDTA. Pharmaceutical carriers also include ethyl alcohol, polyethylene glycol and propylene glycol for water miscible vehicles, and sodium hydroxide, hydrochloric acid, citric acid, or lactic acid for pH adjustment.

Injectables are designed for local and systemic administration. Typically a therapeutically effective dosage is formulated to contain a concentration of at least about 0.1% w/w up to about 90% w/w or more, such as more than 1% w/w of the active compound to the treated tissue(s). The active ingredient may be administered at once, or may be divided into a number of smaller doses to be administered at intervals of time. It is understood that the precise dosage and duration of treatment is a function of the tissue being treated and may be determined empirically using known testing protocols or by extrapolation from in vivo or in vitro test data. It is to be noted that concentrations and dosage values may also vary with the age of the individual treated. It is to be further understood that for any particular subject, specific dosage regimens should be adjusted over time according to the individual need and the professional judgment of the person administering or supervising the administration of the formulations, and that the concentration ranges set forth herein are exemplary only and are not intended to limit the scope or practice of the claimed formulations.

5.8.3 Lyophilized Powders

Of interest herein are also lyophilized powders, which can be reconstituted for administration as solutions, emulsions, and other mixtures. They may also be reconstituted and formulated as solids or gels.

The sterile, lyophilized powder is prepared by dissolving a compound provided herein, or a pharmaceutically acceptable salt thereof, in a suitable solvent. The solvent may contain an excipient which improves the stability or other pharmacological component of the powder or reconstituted solution, prepared from the powder. Excipients that may be used include, but are not limited to, dextrose, sorbital, fructose, corn syrup, xylitol, glycerin, glucose, sucrose, or other suitable agent. The solvent may also contain a buffer, such as citrate, phosphate, or other buffers known to those of skill in the art. In one embodiment, the buffer has a pH about neutral. Subsequent sterile filtration of the solution followed by lyophilization under standard conditions known to those of skill in the art provides the desired formulation. Generally, the resulting solution will be apportioned into vials for lyophilization. Each vial will contain a single dosage (including but not limited to 10-1000 mg or 100-500 mg) or multiple dosages of the compound. The lyophilized powder can be stored under appropriate conditions, such as at about 4° C. to room temperature.

Reconstitution of this lyophilized powder with water for injection provides a formulation for use in parenteral administration. For reconstitution, about 1-50 mg, about 5-35 mg, or about 9-30 mg of lyophilized powder, is added per milliliter of sterile water or other suitable carrier. The precise amount depends upon the selected compound. Such amount can be empirically determined.

5.8.4 Topical Administration

Topical mixtures are prepared as described for the local and systemic administration. The resulting mixture may be a solution, suspension, emulsion, or the like and are formulated as creams, gels, ointments, emulsions, solutions, elixirs, lotions, suspensions, tinctures, pastes, foams, aerosols, irrigations, sprays, suppositories, bandages, dermal patches, or any other formulations suitable for topical administration.

The compounds or pharmaceutically acceptable salts thereof may be formulated as aerosols for topical application, such as by inhalation (see, e.g., U.S. Pat. Nos. 4,044,126, 4,414,209, and 4,364,923, which describe aerosols for delivery of a steroid useful for treatment of inflammatory diseases, particularly asthma). These formulations for administration to the respiratory tract can be in the form of an aerosol or solution for a nebulizer, or as a microfine powder for insufflation, alone or in combination with an inert carrier such as lactose. In such a case, the particles of the formulation will have diameters of less than 50 microns or less than 10 microns.

These solutions, particularly those intended for ophthalmic use, may be formulated as 0.01%-10% isotonic solutions, pH about 5-7, with appropriate salts. 5.8.5 Compositions for Other Routes of Administration

Other routes of administration such as transdermal patches and rectal administration are also contemplated herein.

For example, pharmaceutical dosage forms for rectal administration are rectal suppositories, capsules, and tablets for systemic effect. Rectal suppositories as used herein mean solid bodies for insertion into the rectum, which melt or soften at body temperature releasing one or more pharmacologically or therapeutically active ingredients. Pharmaceutically acceptable substances utilized in rectal suppositories include bases (or vehicles) and agents that raise the melting point. Examples of bases include, for example, cocoa butter (theobroma oil), glycerin gelatin, carbowax (polyoxyethylene glycol), and appropriate mixtures of mono, di and triglycerides of fatty acids. Combinations of the various bases may be used. Agents to raise the melting point of suppositories include, for example, spermaceti and wax. Rectal suppositories may be prepared either by the compressed method or by molding. An exemplary weight of a rectal suppository is about 2 to 3 grams.

Tablets and capsules for rectal administration are manufactured using the same pharmaceutically acceptable substance and by the same methods as for formulations for oral administration.

5.8.6 Sustained Release Compositions

Active ingredients provided herein can be administered by controlled release means or by delivery devices that are well known to those of ordinary skill in the art. Examples include, but are not limited to, those described in U.S. Pat. Nos. 3,845,770, 3,916,899, 3,536,809, 3,598,123, 4,008,719, 5,674,533, 5,059,595, 5,591,767, 5,120,548, 5,073,543, 5,639,476, 5,354,556, 5,639,480, 5,733,566, 5,739,108, 5,891,474, 5,922,356, 5,972,891, 5,980,945, 5,993,855, 6,045,830, 6,087,324, 6,113,943, 6,197,350, 6,248,363, 6,264,970, 6,267,981, 6,376,461, 6,419,961, 6,589,548, 6,613,358, 6,699,500, and 6,740,634, each of which is incorporated herein by reference. Such dosage forms can be used to provide slow or controlled-release of one or more active ingredients using, for example, hydropropylmethyl cellulose, other polymer matrices, gels, permeable membranes, osmotic systems, multilayer coatings, microparticles, liposomes, microspheres, or a combination thereof, to provide the desired release profile in varying proportions. Suitable controlled-release formulations known to those of ordinary skill in the art, including those described herein, can be readily selected for use with the active ingredients provided herein.

All controlled-release pharmaceutical products have a common goal of improving drug therapy over their non-controlled counterparts. In one embodiment, the use of an optimally designed controlled-release preparation in medical treatment is characterized by a minimum of drug substance being employed to cure or control the condition in a minimum amount of time. In certain embodiments, advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side effects (e.g., adverse effects).

Most controlled-release formulations are designed to initially release an amount of drug (active ingredient) that promptly produces the desired therapeutic effect, then to gradually and continually release other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water, other physiological conditions, or compounds.

In certain embodiments, the agent may be administered using intravenous infusion, an implantable osmotic pump, a transdermal patch, liposomes, or other modes of administration. In one embodiment, a pump may be used. See, Sefton, CRC Crit. Ref Biomed. Eng. 1987, 14:201-240; Buchwald et al., Surgery 1980, 88:507-516; Saudek et al., N. Engl. J. Med. 1989, 321:574-579. In another embodiment, polymeric materials can be used. In yet another embodiment, a controlled release system can be placed in proximity of the therapeutic target, thus requiring only a fraction of the systemic dose. See, e.g., Goodson, Medical Applications of Controlled Release, vol. 2, pp. 115-138 (1984).

In some embodiments, a controlled release device is introduced into a subject in proximity of the site of inappropriate immune activation or a tumor. Other controlled release systems are discussed in the review by Langer (Science 1990, 249:1527-1533). The active ingredient can be dispersed in a solid inner matrix (e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate). In some embodiments, the inner matrix is surrounded by an outer polymeric membrane (e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene, propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer). In certain embodiments, the outer polymeric membrane is insoluble in body fluids. The active ingredient then diffuses through the outer polymeric membrane in a release rate controlling step. The percentage of active ingredient contained in such parenteral compositions depends on the specific nature thereof, as well as the needs of the subject.

5.8.7 Targeted Formulations

The compounds provided herein, or pharmaceutically acceptable salts thereof, may also be formulated to target a particular tissue, receptor, or other area of the body of the subject to be treated. Many such targeting methods are well known to those of skill in the art. All such targeting methods are contemplated herein for use in the instant compositions. For non-limiting examples of targeting methods, see, e.g., U.S. Pat. Nos. 6,316,652, 6,274,552, 6,271,359, 6,253,872, 6,139,865, 6,131,570, 6,120,751, 6,071,495, 6,060,082, 6,048,736, 6,039,975, 6,004,534, 5,985,307, 5,972,366, 5,900,252, 5,840,674, 5,759,542, and 5,709,874.

In one embodiment, liposomal suspensions, including tissue-targeted liposomes, such as tumor-targeted liposomes, may also be suitable as pharmaceutically acceptable carriers. These may be prepared according to methods known to those skilled in the art. For example, liposome formulations may be prepared as described in U.S. Pat. No. 4,522,811. Briefly, liposomes such as multilamellar vesicles (MLVs) may be formed by drying down egg phosphatidyl choline and brain phosphatidyl serine (7:3 molar ratio) on the inside of a flask. A solution of a compound provided herein in phosphate buffered saline (PBS) lacking divalent cations is added, and the flask is shaken until the lipid film is dispersed. The resulting vesicles are washed to remove unencapsulated compound, pelleted by centrifugation, and then resuspended in PBS.

5.8.8 Articles of Manufacture

The compounds or pharmaceutically acceptable salts can be packaged as articles of manufacture containing packaging material, a compound or pharmaceutically acceptable salt thereof provided herein, which is used for treatment, prevention, or amelioration of one or more symptoms or progression of cancer, including solid cancers and blood borne tumors, and a label indicating that the compound or pharmaceutically acceptable salt thereof is used for treatment, prevention, or amelioration of one or more symptoms or progression of cancer, including solid cancers and blood borne tumors.

The articles of manufacture provided herein contain packaging materials. Packaging materials for use in packaging pharmaceutical products are well known to those of skill in the art. See, e.g., U.S. Pat. Nos. 5,323,907, 5,052,558, and 5,033,252. Examples of pharmaceutical packaging materials include, but are not limited to, blister packs, bottles, tubes, inhalers, pumps, bags, vials, containers, syringes, pens, bottles, and any packaging material suitable for a selected formulation and intended mode of administration and treatment. A wide array of formulations of the compounds and compositions provided herein are contemplated.

### 5.9 Kits for Detecting Biomarker Levels

In certain embodiments, provided herein is a kit for detecting the mRNA level of one or more biomarkers. In certain embodiments, the kit comprises one or more probes that bind specifically to the mRNAs of the one or more biomarkers. In certain embodiments, the kit further comprises a washing solution. In certain embodiments, the kit further comprises reagents for performing a hybridization assay, mRNA isolation or purification means, detection means, as well as positive and negative controls. In certain embodiments, the kit further comprises an instruction for using the kit. The kit can be tailored for in-home use, clinical use, or research use.

In certain embodiments, provided herein is a kit for detecting the protein level of one or more biomarkers. In certain embodiments, the kits comprises a dipstick coated with an antibody that recognizes the protein biomarker, washing solutions, reagents for performing the assay, protein isolation or purification means, detection means, as well as positive and negative controls. In certain embodiments, the kit further comprises an instruction for using the kit. The kit can be tailored for in-home use, clinical use, or research use.

Such a kit can employ, for example, a dipstick, a membrane, a chip, a disk, a test strip, a filter, a microsphere, a slide, a multi-well plate, or an optical fiber. The solid support of the kit can be, for example, a plastic, silicon, a metal, a resin, glass, a membrane, a particle, a precipitate, a gel, a polymer, a sheet, a sphere, a polysaccharide, a capillary, a film, a plate, or a slide. The biological sample can be, for example, a cell culture, a cell line, a tissue, an organ, an organelle, a biological fluid, a blood sample, a urine sample, or a skin sample.

In another embodiment, the kit comprises a solid support, nucleic acids attached to the support, where the nucleic acids are complementary to at least 20, 50, 100, 200, 350, or more bases of mRNA, and a means for detecting the expression of the mRNA in a biological sample.

In a specific embodiment, the pharmaceutical or assay kit comprises, in a container, a compound or a pharmaceutical composition thereof, and further comprises, in one or more containers, components for isolating RNA. In another specific embodiment, the pharmaceutical or assay kit comprises, in a container, a compound or a pharmaceutical composition, and further comprises, in one or more containers, components for conducting RT-PCR, qRT-PCR, deep sequencing, or microarray

In certain embodiments, the kits provided herein employ means for detecting the expression of a biomarker by quantitative real-time PCR (qRT-PCR), microarray, flow cytometry, or immunofluorescence. In other embodiments, the expression of the biomarker is measured by ELISA-based methodologies or other similar methods known in the art.

In another specific embodiment, the pharmaceutical or assay kit comprises, in a container, a compound or a pharmaceutical composition thereof, and further comprises, in one or more containers, components for isolating protein. In another specific embodiment, the pharmaceutical or assay kit comprises, in a container, a compound or a pharmaceutical composition, and further comprises, in one or more containers, components for conducting flow cytometry or ELISA.

In another aspect, provided herein are kits for measuring biomarkers that supply the materials necessary to measure the abundance of one or more gene products of the biomarkers or a subset of the biomarkers (e.g., one, two, three, four, five, or more biomarkers) provided herein. Such kits may comprise materials and reagents required for measuring RNA or protein. In some embodiments, such kits include microarrays, wherein the microarray is comprised of oligonucleotides and/or DNA and/or RNA fragments which hybridize to one or more gene products of the biomarkers or a subset of the biomarkers provided herein, or any combination thereof. In some embodiments, such kits may include primers for PCR of either the RNA product or the cDNA copy of the RNA product of the biomarkers or a subset of the biomarkers, or both. In some embodiments, such kits may include primers for PCR as well as probes for qPCR. In some embodiments, such kits may include multiple primers and multiple probes, wherein some of the probes have different fluorophores so as to permit simultaneously measuring multiple gene products of the biomarkers or a subset of the biomarkers provided herein. In some embodiments, such kits may further include materials and reagents for creating cDNA from RNA. In some embodiments, such kits may include antibodies specific for the protein products of the biomarkers or a subset of the biomarkers provided herein. Such kits may additionally comprise materials and reagents for isolating RNA and/or proteins from a biological sample. In addition, such kits may include materials and reagents for synthesizing cDNA from RNA isolated from a biological sample. In some embodiments, such kits may include a computer program product embedded on computer readable media for predicting whether a patient is clinically sensitive to a compound. In some embodiments, the kits may include a computer program product embedded on a computer readable media along with instructions.

In some embodiments, such kits measure the expression of one or more nucleic acid products of the biomarkers or a subset of the biomarkers provided herein. In accordance with this embodiment, the kits may comprise materials and reagents that are necessary for measuring the expression of particular nucleic acid products of the biomarkers or a subset of the biomarkers provided herein. For example, a microarray or RT-PCR kit may be produced for a specific condition and contain only those reagents and materials necessary for measuring the levels of specific RNA transcript products of the biomarkers or a subset of the biomarkers provided herein, to predict whether a hematological cancer in a patient is clinically sensitive to a compound. Alternatively, in some embodiments, the kits can comprise materials and reagents necessary for measuring the expression of particular nucleic acid products of genes other than the biomarkers provided herein. For example, in certain embodiments, the kits comprise materials and reagents necessary for measuring the expression levels of 1, 2, 3, 4, 5, 6, 7, 8, 9, 10, 15, 20, 25, 30, 35, 40, 45, 50, or more of the genes of the biomarkers provided herein, in addition to reagents and materials necessary for measuring the expression levels of at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, or more genes other than the biomarkers provided herein. In other embodiments, the kits contain reagents and materials necessary for measuring the expression levels of at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, or more of the biomarkers provided herein, and 1, 2, 3, 4, 5, 10, 15, 20, 25, 30, 35, 40, 45, 50, 55, 60, 65, 70, 75, 80, 85, 90, 95, 100, 125, 150, 175, 200, 225, 250, 300, 350, 400, 450, or more genes that are not the biomarkers provided herein. In certain embodiments, the kits contain reagents and materials necessary for measuring the expression levels of at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, or more of the genes of the biomarkers provided herein, and 1-10, 1-100, 1-150, 1-200, 1-300, 1-400, 1-500, 1-1000, 25-100, 25-200, 25-300, 25-400, 25-500, 25-1000, 100-150, 100-200, 100-300, 100-400, 100-500, 100-1000 or 500-1000 genes that are not the biomarkers provided herein.

For nucleic acid microarray kits, the kits generally comprise probes attached to a solid support surface. In one such embodiment, probes can be either oligonucleotides or longer probes including probes ranging from 150 nucleotides to 800 nucleotides in length. The probes may be labeled with a detectable label. In a specific embodiment, the probes are specific for one or more of the gene products of the biomarkers provided herein. The microarray kits may comprise instructions for performing the assay and methods for interpreting and analyzing the data resulting from performing the assay. In a specific embodiment, the kits comprise instructions for predicting whether a hematological cancer in a patient is clinically sensitive to a compound. The kits may also comprise hybridization reagents and/or reagents necessary for detecting a signal produced when a probe hybridizes to a target nucleic acid sequence. Generally, the materials and reagents for the microarray kits are in one or more containers. Each component of the kit is generally in its own suitable container.

In certain embodiments, a nucleic acid microarray kit comprises materials and reagents necessary for measuring the expression levels of 1, 2, 3, 4, 5, 6, 7, 8, 9, 10, 15, 20, 25, 30, 35, 40, 45, 50, or more of the genes of the biomarkers provided herein, or a combination thereof, in addition to reagents and materials necessary for measuring the expression levels of at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, or more genes other than those of the biomarkers provided herein. In other embodiments, a nucleic acid microarray kit contains reagents and materials necessary for measuring the expression levels of at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, or more of the genes of the biomarkers provided herein, or any combination thereof, and 1, 2, 3, 4, 5, 10, 15, 20, 25, 30, 35, 40, 45, 50, 55, 60, 65, 70, 75, 80, 85, 90, 95, 100, 125, 150, 175, 200, 225, 250, 300, 350, 400, 450, or more genes that are not of the biomarkers provided herein. In another embodiment, a nucleic acid microarray kit contains reagents and materials necessary for measuring the expression levels of at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, or more of the genes of the biomarkers provided herein, or any combination thereof, and 1-10, 1-100, 1-150, 1-200, 1-300, 1-400, 1-500, 1-1000, 25-100, 25-200, 25-300, 25-400, 25-500, 25-1000, 100-150, 100-200, 100-300, 100-400, 100-500, 100-1000, or 500-1000 genes that are not of the biomarkers provided herein.

For quantitative PCR, the kits generally comprise pre-selected primers specific for particular nucleic acid sequences. The quantitative PCR kits may also comprise enzymes suitable for amplifying nucleic acids (e.g., polymerases such as Taq polymerase), deoxynucleotides, and buffers needed for amplification reaction. The quantitative PCR kits may also comprise probes specific for the nucleic acid sequences associated with or indicative of a condition. The probes may or may not be labeled with a fluorophore. The probes may or may not be labeled with a quencher molecule. In some embodiments, the quantitative PCR kits also comprise components suitable for reverse-transcribing RNA, including enzymes (e.g., reverse transcriptases such as AMV, MMLV, and the like) and primers for reverse transcription along with deoxynucleotides and buffers needed for reverse transcription reaction. Each component of the quantitative PCR kit is generally in its own suitable container. Thus, these kits generally comprise distinct containers suitable for each individual reagent, enzyme, primer and probe. Further, the quantitative PCR kits may comprise instructions for performing the reaction and methods for interpreting and analyzing the data resulting from performing the reaction. In a specific embodiment, the kits contain instructions for predicting whether a hematological cancer in a patient is clinically sensitive to a compound.

For antibody-based kits, the kit can comprise, for example: (1) a first antibody (which may or may not be attached to a solid support) that binds to a peptide, polypeptide or protein of interest; and, optionally, (2) a second, different antibody that binds to either the first antibody or the peptide, polypeptide, or protein, and is conjugated to a detectable label (e.g., a fluorescent label, radioactive isotope, or enzyme). In a specific embodiment, the peptide, polypeptide, or protein of interest is associated with or indicative of a condition (e.g., a disease). The antibody-based kits may also comprise beads for conducting immunoprecipitation. Each component of the antibody-based kits is generally in its own suitable container. Thus, these kits generally comprise distinct containers suitable for each antibody and reagent. Further, the antibody-based kits may comprise instructions for performing the assay and methods for interpreting and analyzing the data resulting from performing the assay. In a specific embodiment, the kits contain instructions for predicting whether a hematological cancer in a patient is clinically sensitive to a compound.

In one embodiment, a kit provided herein comprises a compound provided herein, or a pharmaceutically acceptable salt, solvate, or hydrate thereof. Kits may further comprise additional active agents, including but not limited to those disclosed herein.

Kits provided herein may further comprise devices that are used to administer the active ingredients. Examples of such devices include, but are not limited to, syringes, drip bags, patches, and inhalers.

Kits may further comprise cells or blood for transplantation, as well as pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to, water for injection USP; aqueous vehicles (such as, but not limited to, sodium chloride injection, Ringer's injection, dextrose injection, dextrose and sodium chloride injection, and lactated Ringer's injection); water-miscible vehicles (such as, but not limited to, ethyl alcohol, polyethylene glycol, and polypropylene glycol); and non-aqueous vehicles (such as, but not limited to, corn oil, cottonseed oil, peanut oil, sesame oil, ethyl oleate, isopropyl myristate, and benzyl benzoate).

In certain embodiments of the methods and kits provided herein, solid phase supports are used for purifying proteins, labeling samples, or carrying out the solid phase assays. Examples of solid phases suitable for carrying out the methods disclosed herein include beads, particles, colloids, single surfaces, tubes, multi-well plates, microtiter plates, slides, membranes, gels, and electrodes. When the solid phase is a particulate material (e.g., a bead), it is, in one embodiment, distributed in the wells of multi-well plates to allow for parallel processing of the solid phase supports.

It is noted that any combination of the above-listed embodiments, for example, with respect to one or more reagents, such as, without limitation, nucleic acid primers, solid support, and the like, are also contemplated in relation to any of the various methods and/or kits provided herein.

Certain embodiments of the invention are illustrated by the following non-limiting examples.

### 6. EXAMPLES

The examples below are carried out using standard techniques, which are well known and routine to those of skill in the art, except where otherwise described in detail. The examples are intended to be merely illustrative.

### 6.1 Preparation of 1-(3-chloro-4-methylphenyl)-3-((2-(2,6-dioxopiperidin-3-yl)-1-oxoisoindolin-5-yl)methyl)urea (Compound C)

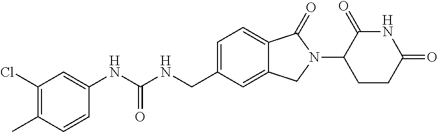 [SMILES: Cc1ccc(NC(=O)NCc2ccc3c(c2)CN(C2CCC(=O)NC2=O)C3=O)cc1Cl]

Step 1:

A mechanically stirred mixture of 4-bromo-2-methyl-benzoic acid (100 g, 465 mmol), iodomethane (95 g, 670 mmol) and sodium bicarbonate (112 g, 1340 mmol) in DMF (325 mL) was heated at 80° C. overnight. The reaction mixture was cooled to room temperature and partitioned between water (1500 mL) and 4:1 hexanes:ethyl acetate (1500 mL). The organic layer was washed with water and dried (Na₂SO₄). The solvent was removed under vacuum to give 110 g of 4-bromo-2-methyl-benzoic acid methyl ester as an oil, in 100% yield; 1H NMR (DMSO-d6) δ 2.51 (s, 3H), 3.84 (s, 3H), 7.40-7.78 (m, 3H).

Step 2:

A mechanically stirred mixture of 4-bromo-2-methyl-benzoic acid methyl ester (115 g, 500 mmol), N-bromosuccinimide (90 g, 500 mmol) and AIBN (3.1 g) in acetonitrile (700 mL) was warmed over 45 minutes to a gentle reflux, and held at reflux for 21 hours. The reaction mixture was cooled to room temperature, diluted with saturated aqueous sodium bisulfite, and concentrated in vacuo. The residue was partitioned between water and 1:1 hexanes:ethyl acetate. The organic phase was washed with water, brine, and filtered through a pad of silica gel. The solvent was removed under vacuum to give an oil/solid mixture, which was digested in ether and filtered. The filtrate was chromatographed on silica gel using a hexanes-ethyl acetate gradient, eluting the product at 4:1 hexanes-ethyl acetate and providing 102 g of 4-bromo-2-bromomethyl-benzoic acid methyl ester, in 66% yield; 1H NMR (DMSO-d6) δ 3.87 (s, 3H), 4.99 (s, 2H), 7.67-7.97 (m, 3H).

Step 3:

A mechanically stirred mixture of 4-bromo-2-bromomethyl-benzoic acid methyl ester (121 g, 390 mmol) and 3-amino-piperidine-2,6-dione hydrochloride (64.2 g, 390 mmol) in DMF (400 mL) was treated dropwise with triethylamine (98.5 g, 980 mmol) over 75 minutes. After the addition was completed, the reaction mixture was stirred at room temperature overnight. The mixture was quenched sequentially with acetic acid (50 mL), water (2500 mL) and a 1:1 mixture of ethyl acetate and hexanes (600 mL). After stirring the mixture for 20 minutes, the solid was filtered, washed with water, and air dried overnight. The solid was stirred in acetic acid (200 mL) and refluxed for 2 hours. The mixture was cooled to room temperature and filtered. The solid was washed with additional acetic acid, hexanes, and air dried overnight to give 25.4 g of 3-(5-bromo-1-oxo-1,3-dihydro-isoindol-2-yl)-piperidine-2,6-dione as a grey solid, in 20% yield; 1H NMR (DMSO-d6) δ 1.97-2.04 (m, 1H), 2.32-2.46 (m, 1H), 2.56-2.63 (m, 1H), 2.85-2.97 (m, 1H), 4.34 (d, J=17.7 Hz, 1H), 4.47 (d, J=17.7 Hz, 1H), 5.11 (dd, J=13.2 Hz, J=5.1 Hz, 1H), 7.67 (d, J=8.1 Hz, 1H), 7.72 (dd, J=8.1 Hz, J=1.5 Hz, 1H), 7.89 (d, J=0.9 Hz, 1H), 11.00 (s, 1H).

Step 4:

A mechanically stirred mixture of 3-(5-bromo-1-oxo-1,3-dihydro-isoindol-2-yl)-piperidine-2,6-dione (25.2 g, 78 mmol), bis(diphenylphosphino)ferrocene (2.0 g), tris(dibenzylideneacetone)dipalladium (2.0 g) and zinc cyanide (9.4 g, 80 mmol) in DMF (300 mL) was heated to 120° C. and stirred at this temperature for 19 hours. The reaction mixture was cooled to 40° C., and another 9.4 g of zinc cyanide, 2 g of bis(diphenylphosphino)ferrocene and 2 g of tris(dibenzylideneacetone)dipalladium were added. The mixture was stirred at 120° C. for 2 hours, cooled to room temperature, and quenched with water (900 mL). The solid was filtered, washed with additional water, and air dried overnight. The solid was stirred in hot acetic acid (200 mL) for 20 minutes. The solid was filtered, washed with additional acetic acid, ethyl acetate and hexanes, and air dried to give 30.8 g of crude 2-(2,6-dioxo-piperidin-3-yl)-1-oxo-2,3-dihydro-1H-isoindole-5-carbonitrile as a gray solid; 1H NMR (DMSO-d6) δ 1.99-2.06 (m, 1H), 2.35-2.45 (m, 1H), 2.57-2.63 (m, 1H), 2.86-2.98 (m, 1H), 4.42 (d, J=17.7 Hz, 1H), 4.55 (d, J=17.7 Hz, 1H), 5.15 (dd, J=13.2 Hz, J=5.1 Hz, 1H), 7.91 (d, J=7.8 Hz, 1H), 7.99 (dd, J=7.8 Hz, J=0.9 Hz, 1H), 8.16 (s, 1H), 11.03 (s, 1H).

Step 5:

A mixture of 2-(2,6-dioxo-piperidin-3-yl)-1-oxo-2,3-dihydro-1H-isoindole-5-carbonitrile (9.2 g, 34 mmol), 10% Pd—C(1.7 g) and concentrated HCl (5.3 g) in N-methylpyrrolidone (300 mL) was hydrogenated at 58 psi overnight. The crude reaction mixture was filtered through Celite, and the catalyst was washed with water. The combined filtrate was concentrated in vacuo, and the product, 3-(5-aminomethyl-1-oxo-1,3-dihydro-isoindol-2-yl)-piperidine-2,6-dione hydrochloride, was isolated by fractional crystallization of the residue from isopropanol-water (1.9 g, 18%); 1H NMR (DMSO-d6) δ 1.85-2.20 (m, 1H), 2.35-2.45 (m, 1H), 2.58-2.80 (m, 1H), 2.87-2.99 (m, 1H), 4.16 (s, 2H), 4.35 (d, J=17.5 Hz, 1H), 4.49 (d, J=17.5 Hz, 1H), 5.13 (dd, J=13.2 Hz, J=4.8 Hz, 1H), 7.63 (d, J=7.8 Hz, 1H), 7.72 (s, 1H), 7.79 (d, J=7.8 Hz, 1H), 8.43 (br, 3H), 11.01 (s, 1H).

Step 6:

A mixture of 3-(5-aminomethyl-1-oxo-1,3-dihydro-isoindol-2-yl)-piperidine-2,6-dione hydrochloride (0.5 g, 1.6 mmol), 3-chloro-4-methylphenyl isocyanate (0.27 g, 1.6 mmol) and TEA (0.32 g, 3.2 mmol) in THF (25 mL) was heated to 40° C. with stirring under N₂. After 3 hours, an additional portion of 3-chloro-4-methylisocyanate (0.17 g, 1.1 mmol) was added, and stirring proceeded for 2 hours. The mixture was filtered, and the filter was washed with ethyl acetate. The solid was triturated with 10 mL of 1:1 acetone-DMF and filtered. The filter was washed with acetone, and the solid was dried under vacuum, providing 430 mg of the product, in 60% yield; mp 258-260° C.; HPLC, Waters Symmetry C-18, 3.9×150 mm, 5 μm, 1 mL/min, 240 nm, 40/60 CH₃CN/0.1% H₃PO₄, 4.49 (98.75%); 1H NMR (DMSO-d6) δ 1.90-1.96 (m, 1H), 2.16 (s, 3H), 2.25-2.39 (m, 1H), 2.50-2.55 (m, 1H), 2.78-2.91 (m, 1H), 4.24 (d, J=18.0 Hz, 1H), 4.33-4.41 (m, 3H), 5.04 (dd, J=13.5 Hz, J=4.5 Hz, 1H), 6.73 (t, J=6.0 Hz, 1H), 7.04-7.13 (m, 2H), 7.36-7.44 (m, 2H), 7.59-7.44 (m, 2H), 8.69 (s, 1H), 10.92 (s, 1H); 13C NMR (DMSO-d6) δ 18.7, 22.5, 31.2, 42.8, 47.1, 51.5, 116.4, 117.6, 121.9, 122.9, 126.9, 127.4, 130.3, 131.0, 133.0, 139.6, 142.4, 144.7, 155.1, 167.9, 171.0, 172.9; Anal. Calcd for C₂₂H₂₁ClN₄O₄: C, 59.93; H, 4.80; N, 12.71. Found: C, 59.77; H, 4.61; N, 12.69.

6.2 Identification of Novel Binding Partners of CBRN Induced by Compound C Binding

Whole cell lysate of 293 HEK cells stably expressing FLAG-HA tagged CRBN was treated with 1 μM Compound C or DMSO vehicle control. Proteins associated with FLAG-HA CRBN were immunoprecipitated with anti-FLAG affinity gel, separated on SDS-PAGE, silver-stained, and analyzed by mass spectrometry. PABP1, GSPT1/eRF3a, GSPT2/eRF3b, and HBS1L/eRF3c were identified as CAPs only when CRBN was bound with Compound C. The left part of FIG. 1 shows the silver staining gel of FLAG-HA CRBN immunoprecipitates. Arrows point to the expected positions of DDB1, GSPT1, PABP1, and CRBN.

Immunblotting analysis was performed to confirm GSPT1/eRF3a, GSPT2/eRF3b, and HBS1L/eRF3c as authentic substrates of the CRBN/Compound C complex. 293 HEK cells transiently transfected with HA-tagged HBS1L or FLAG-tagged GSPT2 were treated with Compound at the indicated concentrations for 8 hours. The right part of FIG. 1 confirms that GSPT1/eRF3a, GSPT2/eRF3b, and HBS1L/eRF3c are binding partners of CRBN induced by Compound C binding. Furthermore, FIG. 1 demonstrates that increased concentration of Compound C induces degradation of the novel binding proteins GSPT1/eRF3a, GSPT2/eRF3b, and HBS1L/eRF3c.

Thus, new binding partners of CRBN induced by Compound C binding, GSPT1/eRF3a, GSPT2/eRF3b, and HBS1L/eRF3c, are identified, and Compound C changes the level of these proteins likely through a CRBN-dependent pathway.

6.3 Compound C Promotes the Interaction between CRBN and Its Substrates IKZF1 or GSPT1/2 In Vitro.

In vitro binding assay was performed to demonstrate that Compound C promotes interaction between CRBN and its substrates. CRBN−/− cells expressing HA-tagged substrates were lysed and incubated with anti-HA antibody to pull down substrates. CRBN positive cells expressing shGSPT1 that specifically knocks down GSPT1 were lysed, then mixed with the substrates obtained from CRBN−/− cells. The mixture was incubated with DMSO alone or compounds. Immunoprecipitation using anti-HA antibody was performed. Then, immunoblotting was performed using anti-CRBN or anti-HA antibodies.

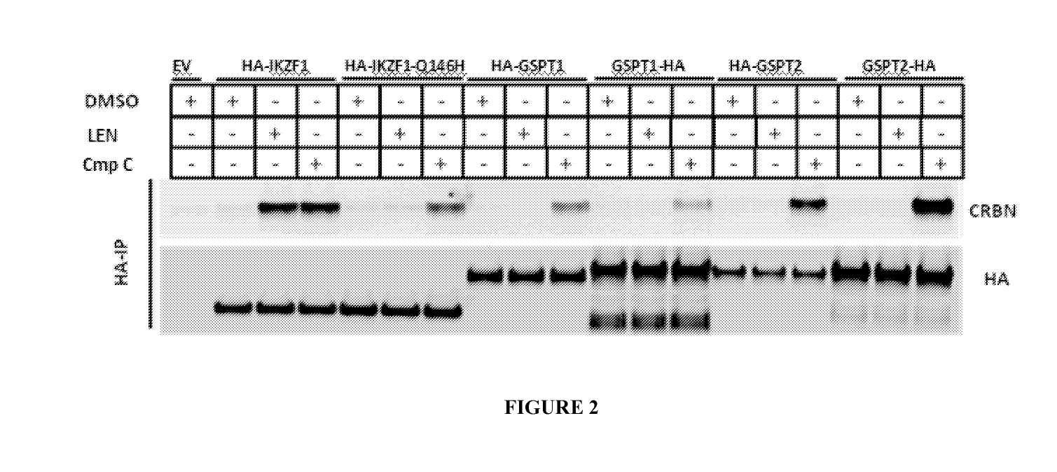

As shown in FIG. 2, Compound C promotes the interaction between CRBN and its substrates IKZF1, GSPT1, or GSPT2. As shown, lenalidomide also promotes the binding of CRBN with its substrate IKZF1, but not with other substrates GSPT1 or GSPT2. The lenalidomide-induced CRBN-IKZF1 interaction is abolished by a specific mutation Q146H in IKZF1.

6.4 GSPT1 Level Reduces in Response to Treatment with Compound C in Lymphoma Cell Line.

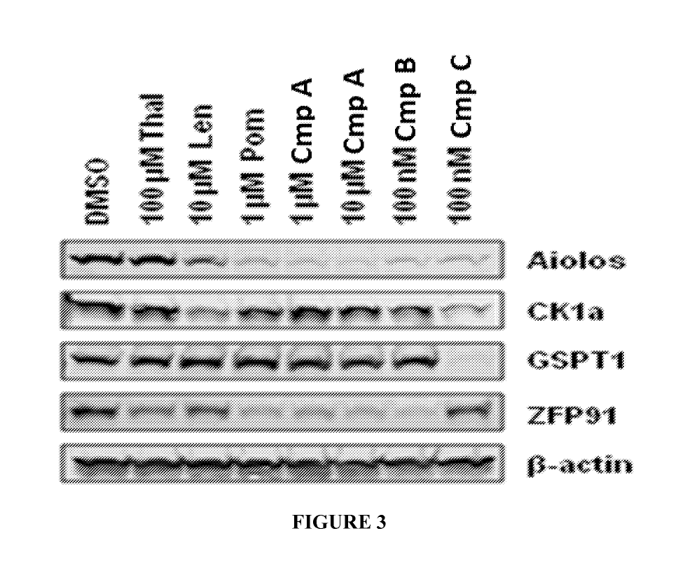

Lymphoma cell line OCI-LY10 was used for Western blot analysis after treatment with DMSO, 100 μM thalidomide, 10 μM lenalidomide, 1 μM pomalidomide, 1 μM Compound A, 10 μM Compound A, 100 μM Compound B, or 100 μM Compound C for 6 hours. Cells were harvested with RIPA buffer, and proteins from cell lysates were separated by 10% sodium dodecyl sulfate polyacrylamide (SDS-PAGE) gel electrophoresis (Bio-Rad), then transferred to PVDF membranes (Invitrogen). Immunoblots were probed with antibodies recognizing Aiolos (9-9-7; Celgene), CK1a (Abcam), GSPT1 (Sigma), ZFP91 (LSBio) and β-actin (Li-Cor). Signals were detected with a Li-Cor Odyssey imager. FIG. 3 shows that GSPT1 protein level reduces in response to the treatment with Compound C but not the other treatment compounds in the lymphoma cell line. In addition, as shown in FIG. 3, Aiolos and CK1a protein levels also reduce in response to the treatment with Compound C.

6.5 Compound C Induces Depletion of GSPT1 and Its Binding Partner eRF1 in 293FT HEK cells.

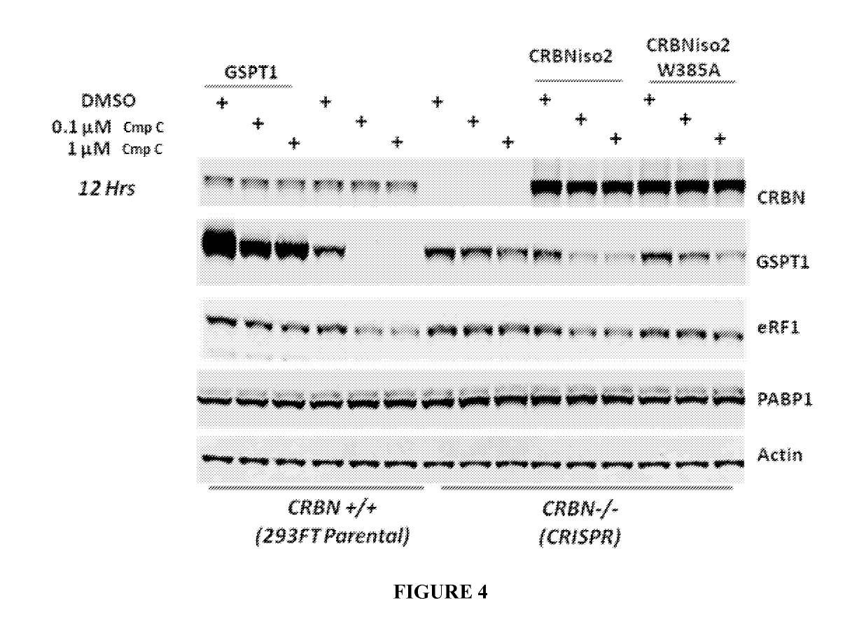

Immunoblotting demonstrates that Compound C induces depletion of GSPT1 and eRF1 in 293FT HEK cells. CRBN+/+(293FT parental) cells were treated in parallel with CRBN−/− (CRISPR) cells, CRBN−/− cells expressing CRBN isoform 2, and CRBN−/− cells expressing CRBN isoform 2 with a W385A mutation. As shown in FIG. 4, Compound C induces degradation of GSPT1 and eRF1 in CRBN+/+ cells but not in CRBN−/− cells. Overexpression of GSPT1 in CRBN+/+ cells reduces this degradation effect. On the other hand, introduction of CRBNiso2 or CRBNiso2 W385A mutant to the CRBN−/− cells restores Compound C-induced degradation of GSPT1 and eRF1, suggesting that Compound C-induced degradation of GSPT1 and eRF1 is CRBN-dependent.

6.6 Identification of Specific Amino Acids in Human CRBN that are Essential for the Destruction of IKZF1/3 or GSPT1/2

Critical amino acids in human CRBN that are essential for the destruction of IKZF1/3 or GSPT1/2 are identified by specific mutation. Each individual substrate of CRBN was tagged differently, for example, IKZF1-V5, FLAG-IKZF3, Myc-GSPT1, or HA-GSPT2. They were co-expressed together with GFP in CRBN −/− cells. These cells also expressed human CRBN isoform 2, or various specific mutants (hCRBNiso2 E376V, hCRBNiso2 V387I, or hCRBNiso2 W385A) by transfection with corresponding DNA. The cells were treated with DMSO alone, 10 μM lenalidomide, or 1 μM Compound C.

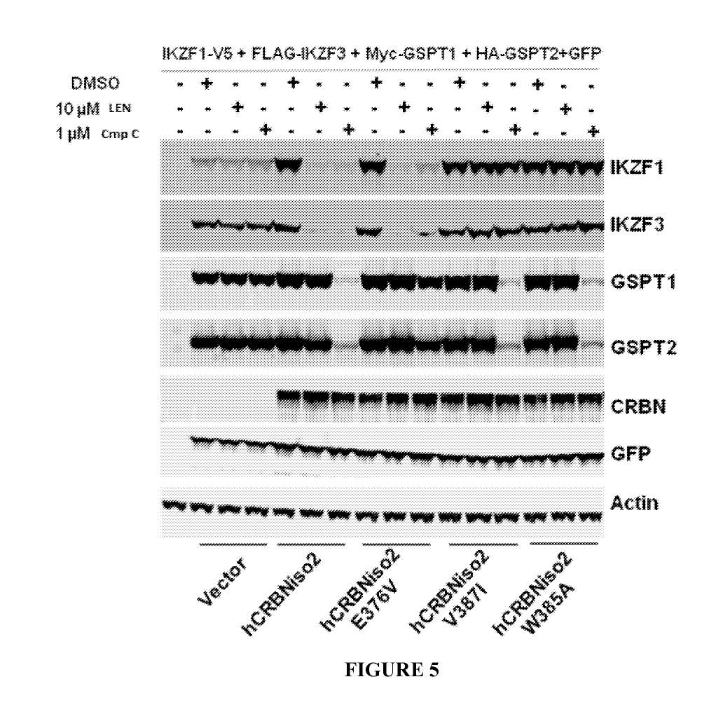

As shown in FIG. 5, without human CRBN, neither compound triggers degradation of any tested substrate. In cells expressing human CRBN isoform 2, Compound C induces destruction of IKZF1/3 and GSPT1/2, whereas lenalidomide triggers destruction of IKZF1/3. Specific mutation E376V in human CRBNiso2 abolishes Compound C-induced degradation of GSPT1/2 but not Compound C-induced degradation of IKZF1/3, suggesting the essential role of E376 in CRBN for the destruction of GSPT1/2. Similarly, specific mutation V387I in human CRBNiso2 abolishes Compound C-induced degradation of IKZF1/3 but not Compound C-induced degradation of GSPT1/2, suggesting the essential role of V387 in CRBN for the destruction of IKZF1/3.

Furthermore, specific mutation W385A in human CRBNiso2 abolishes lenalidomide-induced degradation of IKZF1/3, indicating the essential role of W385 in CRBN for the destruction of IKZF1/3. This is consistent with FIG. 4, which shows that W385A mutation has no effect on the degradation of GSPT1 and eRF1.

6.7 V380E and I391V Mutations are Sufficient to Reactivate Mouse CRBN to Trigger the Degradation of IKZF1/3 and GSPT1/2, Respectively.

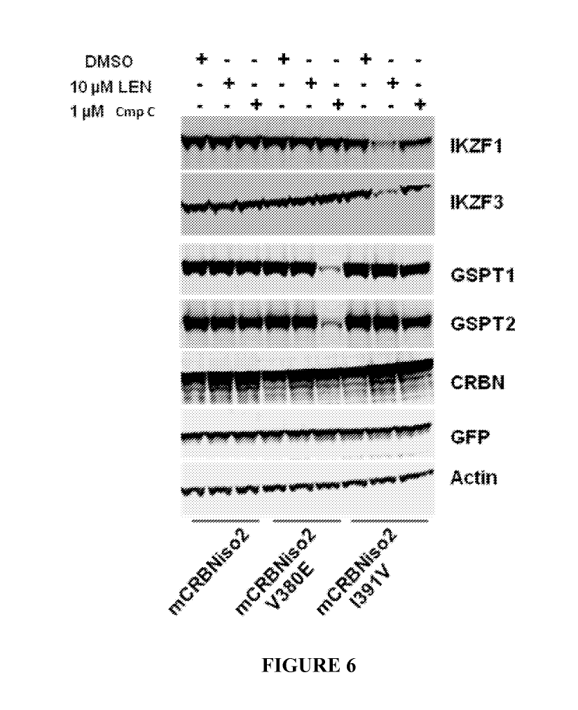

Probably due to variation in the compound-binding domain of mouse CRBN and human CRBN, rodents and humans exhibit differential responses to certain treatment compounds. To test this hypothesis, specific mutations in the compound-binding domain of mouse CRBN isoform2 were generated, such as V380E and I391V. Wild type mouse CRBN isoform 2 or each mutant was introduced to CRBN−/− cells. The cells were treated with DMSO alone, 10 μM lenalidomide, or 1 μM Compound C. As shown in FIG. 6, V380E mutation in mouse CRBN isoform 2 restores Compound C-induced degradation of GSPT1/2, whereas I391V mutation in mouse CRBN isoform 2 restores both lenalidomide- and Compound C-induced degradation of IKZF1/3. Thus, in mouse CRBN isoform 2, V380E and I391V mutations are sufficient to trigger the degradation of IKZF1/3 and GSPT1/2, respectively.

6.8 Overexpression of GSPT1 Confers Compound C Resistance to HEK 293FT Cells.

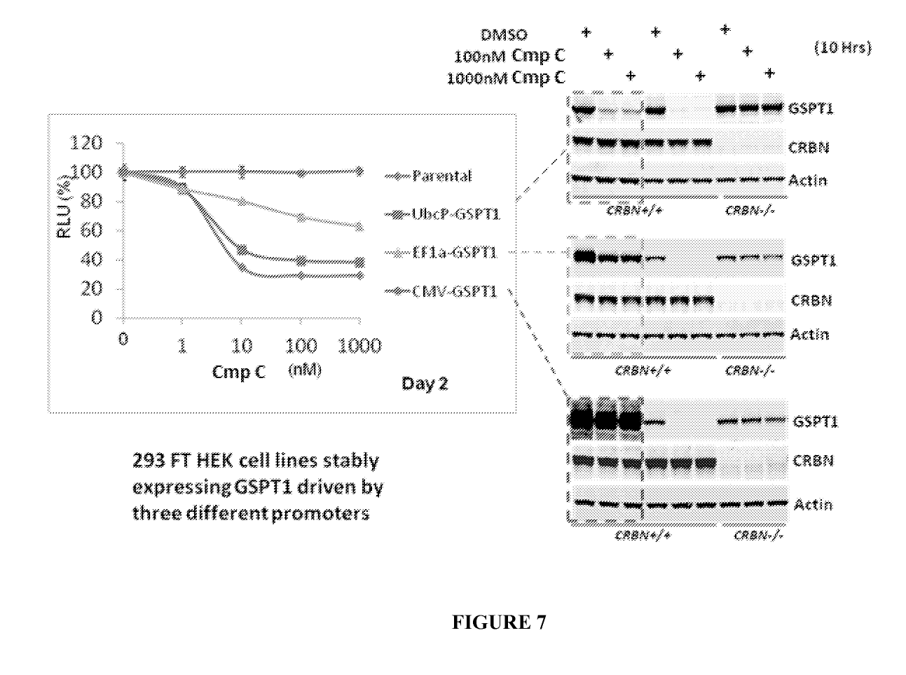

The effect of overexpression of GSPT1 on Compound C-induced growth inhibition was tested in HEK 293FT cells. 293 cells stably expressing GSPT1 driven by three different promoters were generated. To these cells, Compound C was added at concentration of 0, 1 nM, 10 nM, 100 nM, or 1000 nM. Cell proliferation was measured by CellTiter-Glo cell viability assay (RLU-Relative Luminescent Unit) at day 2. As shown in the left part of FIG. 7, Compound C inhibits cell proliferation in parental cells, but overexpression of GSPT1 driven by different promoters confers various degrees of resistance to Compound C-induced growth inhibition. The expression level of GSPT1 was measured at 10 hours after compound treatment in the right part of FIG. 7. Compared to CRBN−/− cells, GSPT1 is degraded in CRBN+/+ cells after 10 hours treatment of 100 nM or 1000 nM Compound C. Furthermore, FIG. 7 shows the highest overexpression level of GSPT1 driven by CMV promoter, followed by EFla and UbcP promoters. These results demonstrate the correlation between overexpression of GSPT1 and cell resistance to Compound C-induced growth inhibition. Cells expressing CMV-GSPT1 exhibit the highest level of Compound C resistance. Thus, overexpression of GSPT1 confers Compound C resistance to HEK 293FT cells.

6.9 Depletion of GSPT1 Inhibits Cell Proliferation.

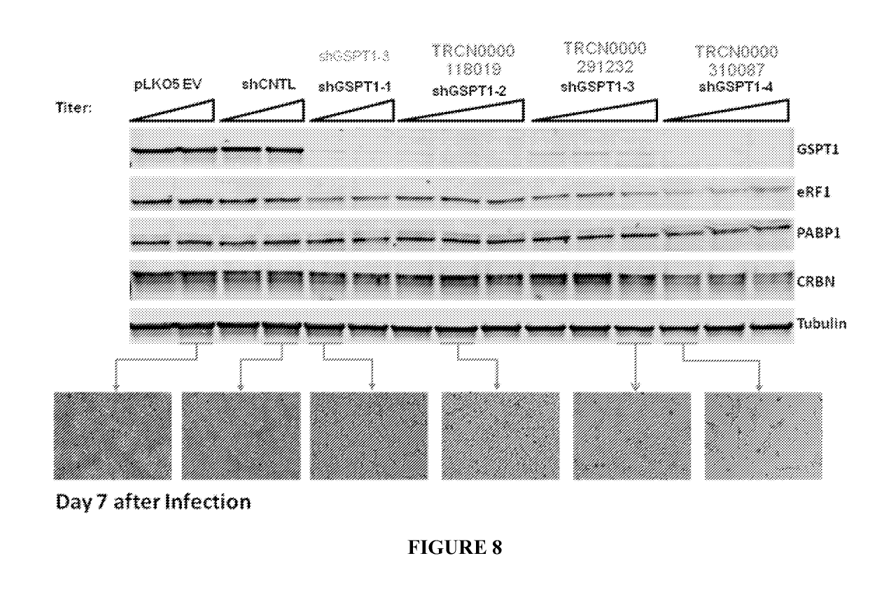

The effect of depletion of GSPT1 (eRF3a) on cell proliferation was determined in 293FT human embryonic kidney cells expressing shRNAs specifically targeting various GSPT1 regions. As demonstrated in FIG. 8, at day 7 after infection, cells with the expression vector alone or control shRNA that is not GSPT1-specific show normal cell proliferation, whereas cells expressing GSPT1-specific shRNAs (such as shGSPT1-1, shGSPT1-2, shGSPT1-3, and shGSPT1-4) show various degrees of inhibition on cell proliferation.

The expression level of various genes in infected cells was also measured in FIG. 8. Compared to the expression vector alone or control shRNA, all four GSPT1-specific shRNAs block the expression of GSPT1. In particular, shGSPT1-4 further inhibits the expression of eRF1 and CRBN.

Thus, depletion of GSPT1 inhibits cell proliferation probably due to the inactivation of the eRF1/GSPT1 (eRF3a) complex.

6.10 Loss of GSPT1 Makes HEK 293FT Cells Susceptible to Compound C-induced Anti-proliferation.

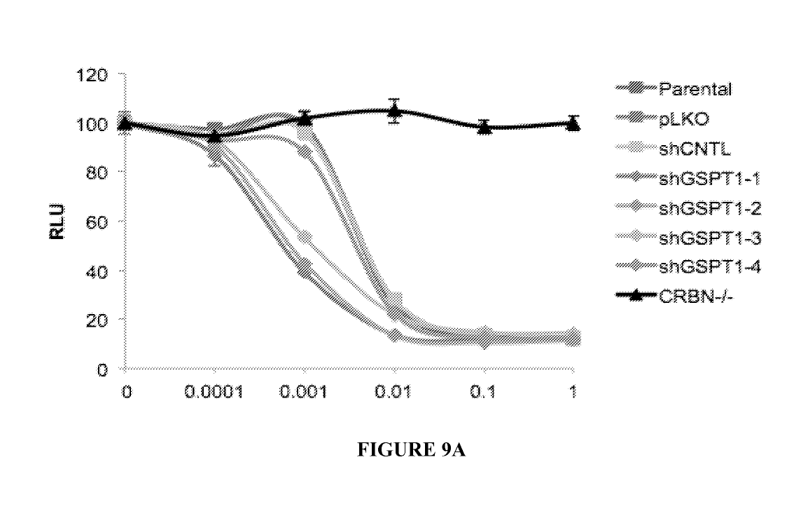

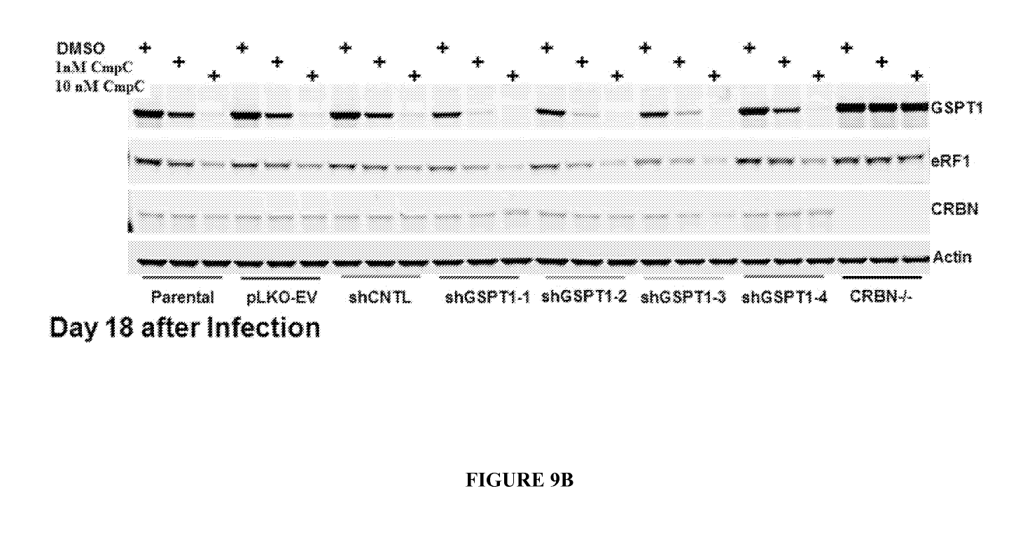

The effect of depletion of GSPT1 on Compound C-induced anti-proliferation was examined in HEK 293FT cells. Cells were infected with either expression vector alone, or vector containing control shRNA or GSPT1-specific shRNA. The cells were then treated with Compound C at different concentrations. Cell proliferation was measured by the CellTiter-Glo cell viability assay (RLU-Relative Luminescent Unit). As shown in FIG. 9A, Compound C cannot inhibit cell proliferation in CRBN−/− cells. Yet CRBN+/+ parental cells, cells infected with expression vector alone, or cells expressing control shRNA that is not specific to GSPT1 show sensitivity to Compound C-induced anti-proliferation. Depletion of GSPT1 by GSPT1-specific shRNA knockdown results in growth inhibition starting at even lower concentration of compound treatment. This result suggests that HEK 293FT cells with depleted GSPT1 have increased sensitivity to Compound C-induced anti-proliferation.

The expression level of GSPT1 and eRF1 was measured at day 18 after infection. As shown in FIG. 9B, the GSPT1-specific shRNA reduces the expression of GSPT1, compared to parental cells, cells with expression vector alone, or cells expressing control shRNA. Compound C induces degradation of GSPT1 and eRF1 in all above cells except CRBN−/− cells. Thus, the increased sensitivity of HEK 293FT cells to Compound C-induced growth inhibition is likely due to depletion of GSPT1.

6.11 Depletion of GSPT1 Sensitizes MM Cell Lines to Compound C-induced Growth Inhibition.

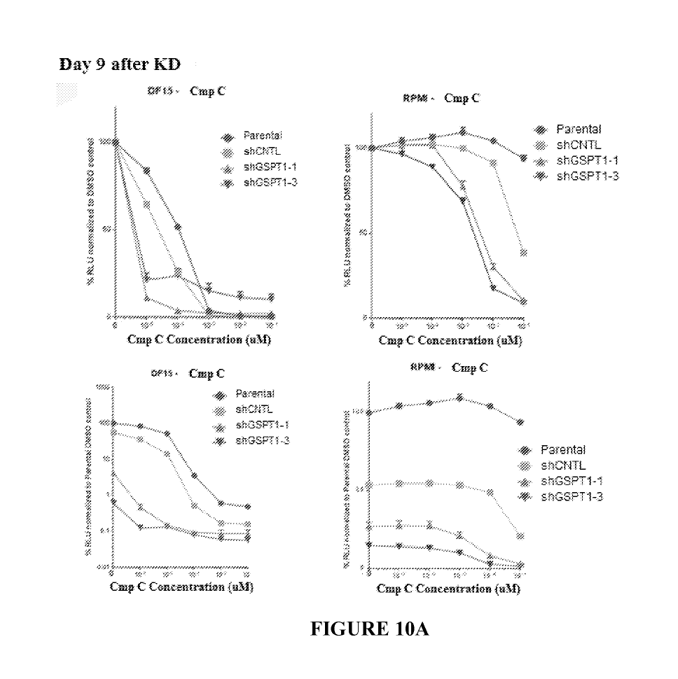

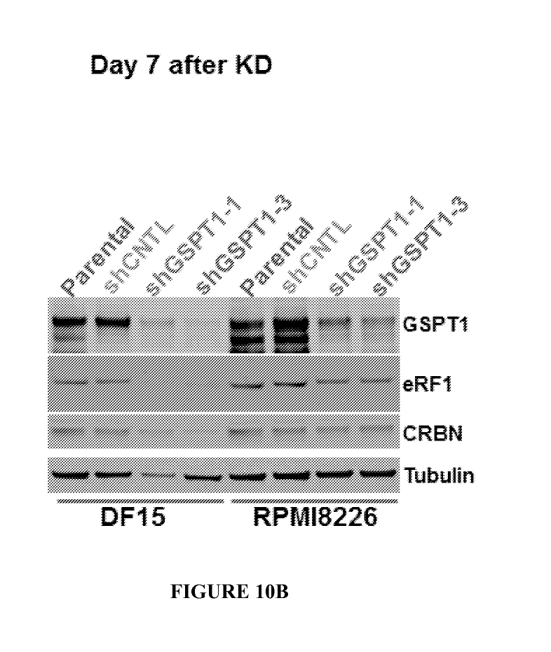

The effect of depletion of GSPT1 on the anti-proliferative effect of Compound C was determined in human multiple myeloma (MM) cell lines DF15 and RPMI-8226. Treatment compounds were titrated from 0.01 nM to 0.1 μM. Cell proliferation was measured by the CellTiter-Glo cell viability assay (RLU-Relative Luminescent Unit) at day 9 after GSPT1 knockdown by shGSPT1-1 or shGSPT1-3. As shown in FIG. 10A, compared to parental cells or cells infected with control shRNA that is not GSPT1-specific, Compound C exhibits increased anti-proliferative effect in cells expressing shGSPT1-1 or shGSPT1-3. As shown in FIG. 10B, this increased sensitivity to Compound C-induced growth inhibition is likely due to depletion of GSPT1 and eRF1.

6.12 Overexpression of GSPT1 Antagonizes the Anti-proliferative Effect of Compound C in U937 and Molm13 Cells.

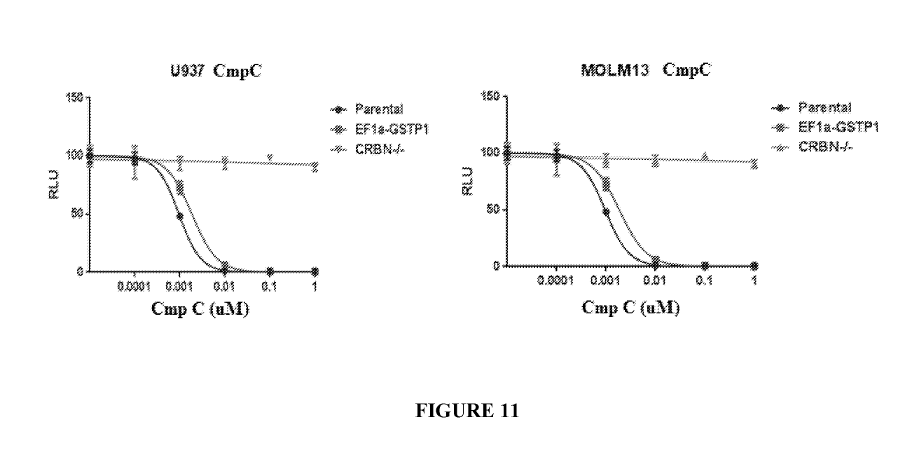

The effect of overexpression of GSPT1 on the anti-proliferative effect of Compound C was determined in human histiocytic lymphoma cell line U937 and human leukemia cell line Molm13. Treatment compounds were titrated from 0.1 nM to 1 μM. Cell proliferation was measured by CellTiter-Glo cell viability assay at 48 hours after treatment. As shown in FIG. 11, in parental cells, Compound C inhibits cell proliferation. Yet in CRBN −/− cells, this anti-proliferative effect is completely abolished, which suggests that the anti-proliferative effect of these compounds is CRBN-dependent. However, when exogenous GSPT1 is overproduced via the EF1a promoter, as shown in FIG. 11, the anti-proliferative effect of Compound C reduces. This result suggests that overexpression of GSPT1 antagonizes the anti-proliferative effect of Compound C in U937 and Molm13 cells.

6.13 Depletion of GSPT1 Sensitizes Acute Myelogenous Leukemia (AML3) Cell Lines to Compound C.

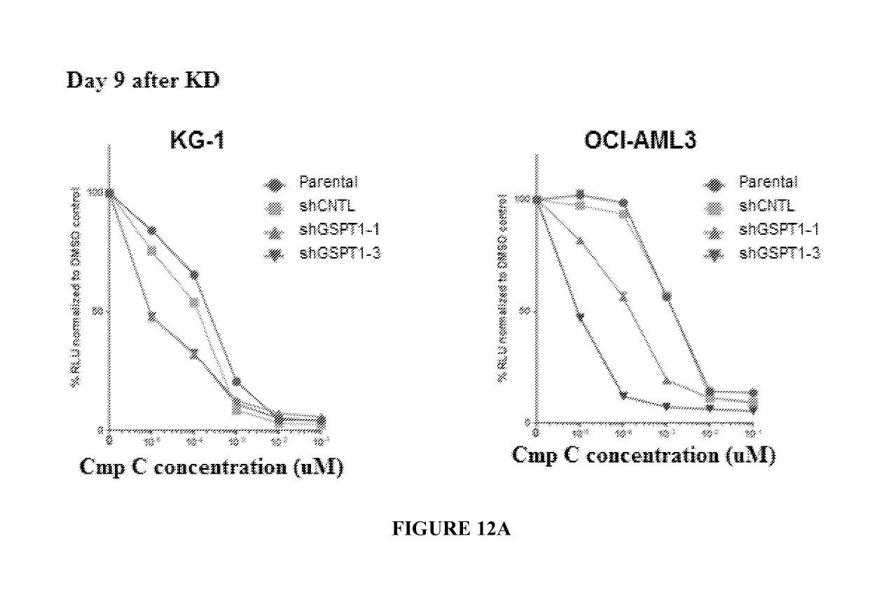

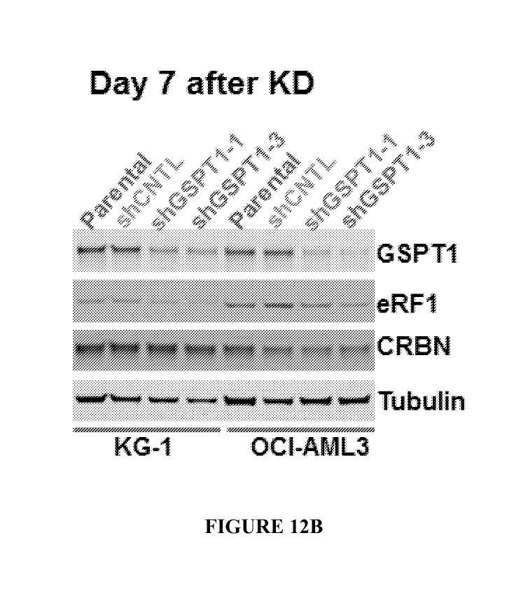

The effect of depletion of GSPT1 on the anti-proliferative effect of Compound C was determined in human Acute Myelogenous Leukemia (AML3) cell line. Cells were infected with lentiviral vectors expressing control shRNA, shGSPT1-1 or shGSPT1-3 for 7 days and then treated with DMSO, Compound C in a titration from 0.0001 μM to 1 μM. Two days after treatment, cell proliferation was measured by CellTiter-Glo cell viability assay. As shown in FIG. 12A, compared to parental cells or cells infected with control shRNA that is not GSPT1-specific, Compound C exhibits increased anti-proliferative effect in cells expressing shGSPT1-1 or shGSPT1-3. As shown in FIG. 12B, this increased sensitivity to Compound C-induced growth inhibition is likely due to depletion of GSPT1 and eRF1.

6.14 Compound C Induces the Activation of the PERK Branch of Unfolded Protein Response (UPR) in 293FT HEK Cells.

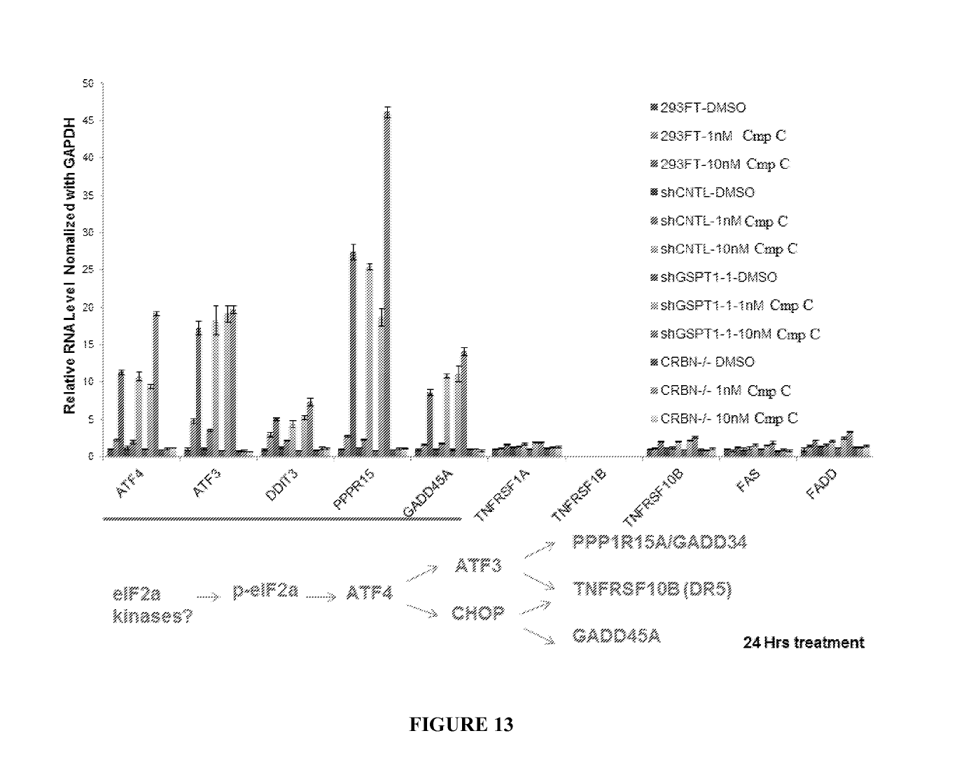

The mechanism of Compound C-induced Unfolded Protein Response (UPR) was studied in 293FT HEK cells. Parental cells, CRBN−/− cells, cells expressing control shRNA, or cells expressing GSPT1-specific shRNA were treated with DMSO, 1 nM, or 10 nM Compound C. The RNA level of variant cellular components along the PERK pathway of UPR was measured and normalized with GAPDH at 24 hours after treatment. As shown in FIG. 13, except in CRBN−/− cells, Compound C induces expression of ATF4, ATF3, DDIT3, PPP1R15A, and GADD45A, which are components along the PERK pathway of UPR. This induction effect increases in cells with GSPT1 knockdown.

6.15 Compound C Activates the XBP1 and ATF6 Pathways in 293FT HEK Cells.

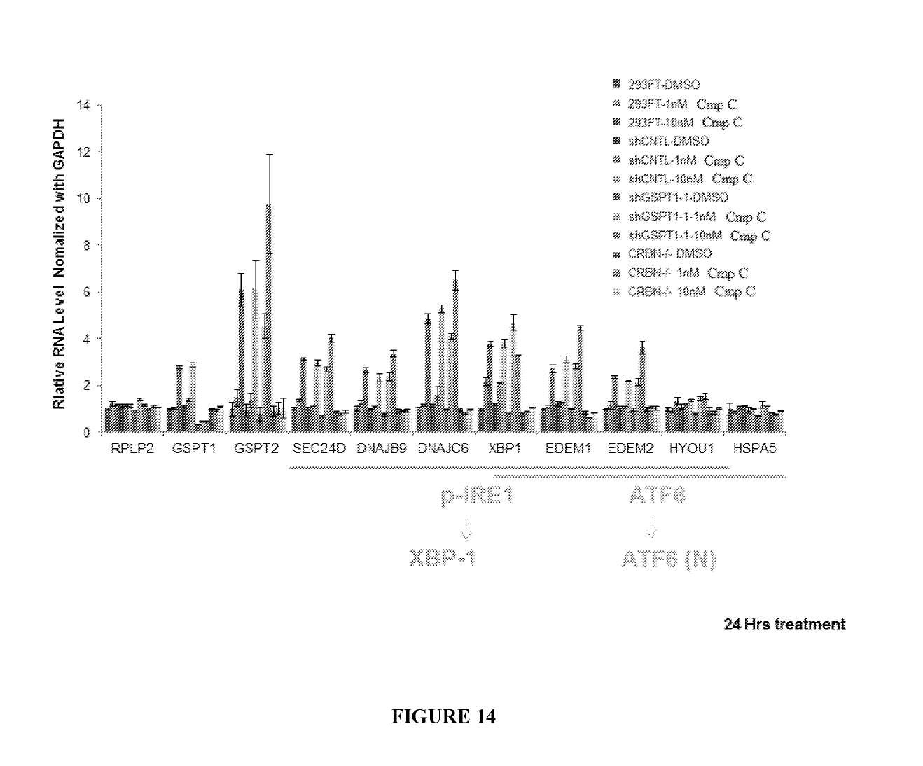

The mechanism of Compound C-activated XBP1 and ATF6 pathways was studied in 293FT HEK cells. Parental cells, CRBN−/− cells, cells expressing control shRNA, or cells expressing GSPT1-specific shRNA were treated with DMSO, 1 nM, or 10 nM Compound C. The RNA level of variant cellular components along the XBP1 and ATF6 pathways was measured and normalized with GAPDH at 24 hours after treatment. As shown in FIG. 14, except in CRBN−/− cells, Compound C induces expression of components along the XBP1 pathway (such as SEC24D, DNAJB9, DNAJC6, XBP1, EDEM1, EDEM2, and HYOU1) and components along the ATF6 pathway (such as XBP1, EDEM1, EDEM2, HYOU1, and HSPA5). This induction effect increases in cells with GSPT1 knockdown.

6.16 Degradation of GSPT1 Leads to Loss of BIP and ER Stress, But Not Acute Apoptotic Cell Death in 293FT HEK Cells.

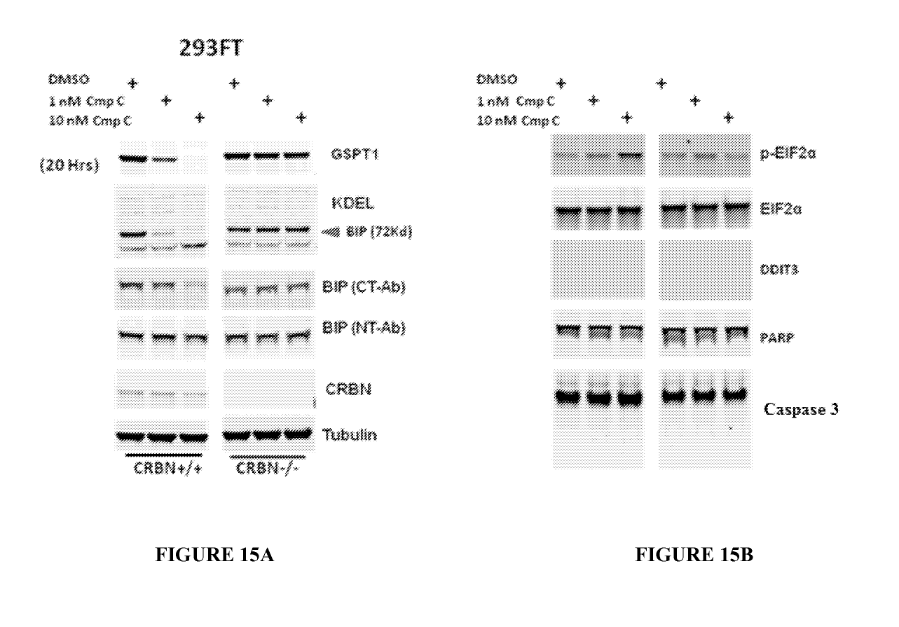

The cellular effect of Compound C-induced degradation of GSPT1 was further studied in 293FT HEK cells. CRBN+/+ and CRBN−/− cells were treated with DMSO alone, 1 nM, or 10 nM Compound C. After 20 hours, the expression level of various cellular components along the endoplasmic reticulum (ER) stress or apoptosis pathways was measured. As shown in FIG. 15A, Compound C induces degradation of GSPT1 in CRBN+/+ cells. BIP is an ER luminal KDEL protein that requires binding with KDEL receptor in the cis-Golgi to be retro-transported into the ER lumen for retention. Loss of GSPT1 leads to the inactivation of the eRF1/GSPT1 complex and therefore allows translation readthrough of de novo sysnthesized proteins such as BIP. Addition of extra residues to the BIP C-terminus blocks the recognization of KDEL motif and BIP C-terminal epitopes by the KDEL receptor and BIP C-terminal antibodies, respectively. Indeed, the immunoreactivity of KDEL antibody as well as BIP antibody that recoginizes BIP carboxyl-terminus (BIP-CT) are dramatically decreased by Compound C treatement in a dose dependent manner. However, BIP antibody that binds to BIP amino-terminal region is not affected. BIP interacts with the ER luminal domain of UPR sensors PERK, IRE1, and ATF6 to prevent their activation. Reduction of BIP C-terminal immunoreactivity indicates a mislocalization of BIP, which presumably leads to its dissociation from PERK, IRE1, and ATF6 and induces UPR. However, as shown in FIG. 15B, cellular components in acute apoptotic cell death are not affected at 20 hours treatment of Compound C in 293FT HEK cells.

6.17 Compound C-Induced UPR Precedes Apoptotic Cell Death in DF15 Cells

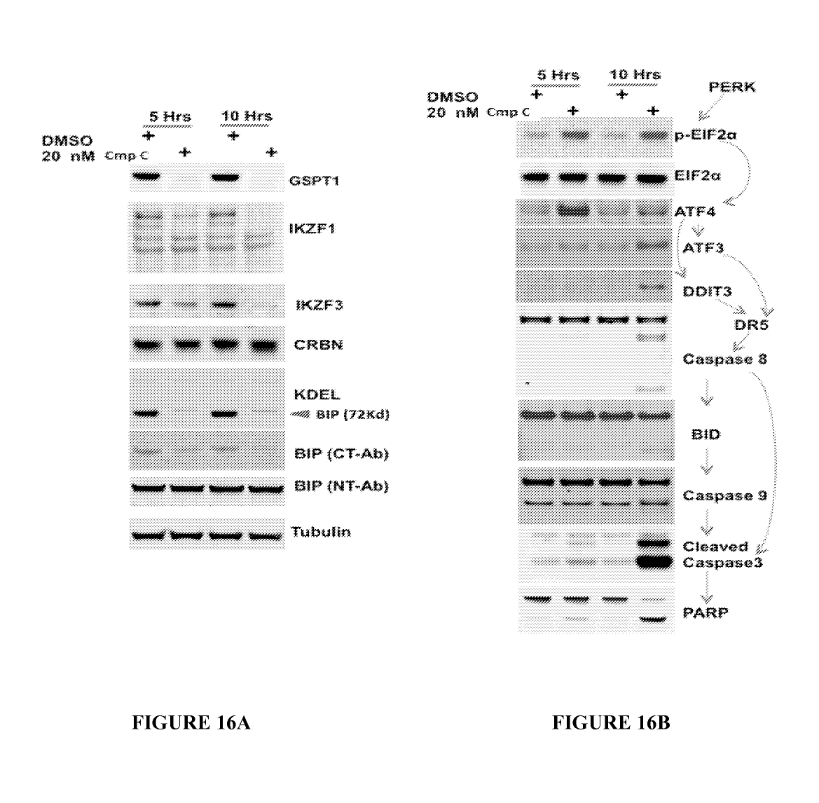

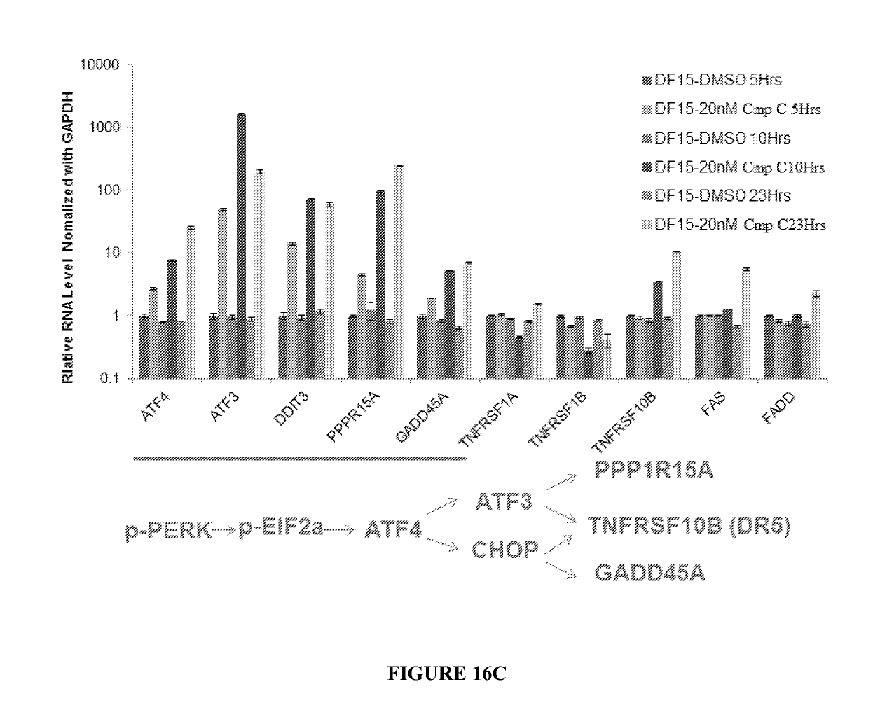

The cellular effect of Compound C-induced degradation of GSPT1 was further studied in DF15 cells. Cells were treated with DMSO alone or 20 nM Compound C. After 5 or 10 hours, the expression level of various cellular components along UPR or apoptosis pathways was measured. As shown in FIG. 16A, Compound C induces degradation of GSPT1, IKZF1, and IKZF3 as well as loss of immunoreactivity of antibodies that recognizes KDEL motif and BIP C-terminal epitope. Loss of BIP immunoreactivity indicates an induction of UPR. Similarly, as shown in FIG. 16B, Compound C increases the level of pEIF2α, ATF4, ATF3, DDIT3, cleaved caspase 3, and cleaved PARP, which suggests the onset of apoptosis. This increase is quantified in FIG. 16C, demonstrating Compound C-induced expression of ATF4, ATF3, DDIT3, PPP1R15A, and GADD45A, components along the PERK/EIF2a/ATF4 pathway in DF15 MM cells.

6.18 Compound C Activates the XBP1 and ATF6 Pathways in DF15 MM Cells.

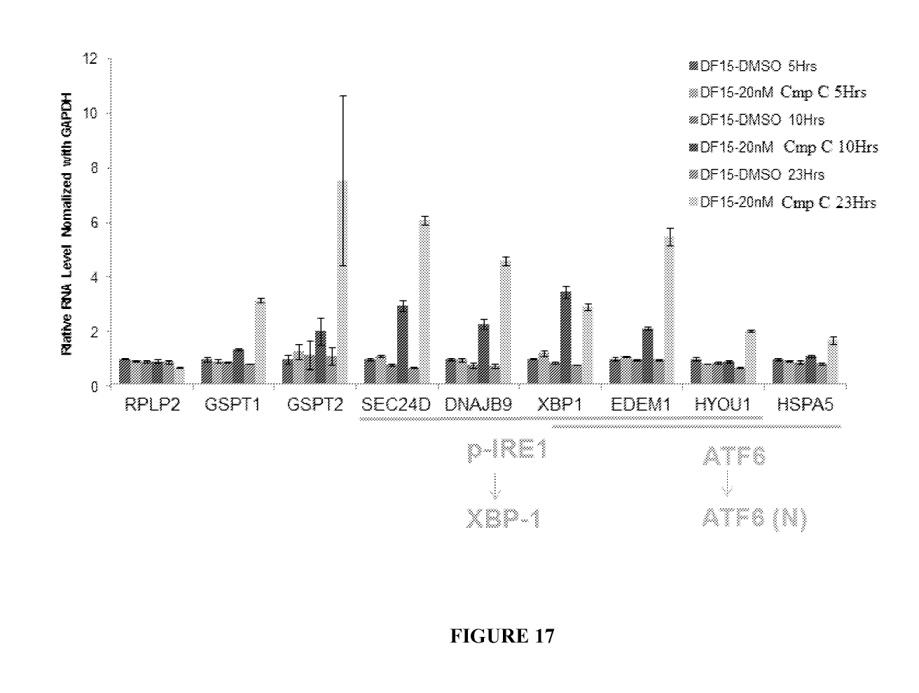

The mechanism of Compound C-activated XBP1 and ATF6 pathways was studied in DF15 MM cells. Cells were treated with DMSO or 20 nM Compound C for 5, 10, or 23 hours. The RNA level of variant cellular components along the XBP1 and ATF6 pathways was measured and normalized with GAPDH. As shown in FIG. 17, Compound C induces expression of components along the XBP1 pathway (such as SEC24D, DNAJB9, XBP1, EDEM1, and HYOU1) and components along the ATF6 pathway (such as XBP1, EDEM1, HYOU1, and HSPA5).

6.19 Compound C-Induced UPR Precedes Apoptotic Cell Death in Human Acute Myeloblastic Leukemia Cell Line KG1.

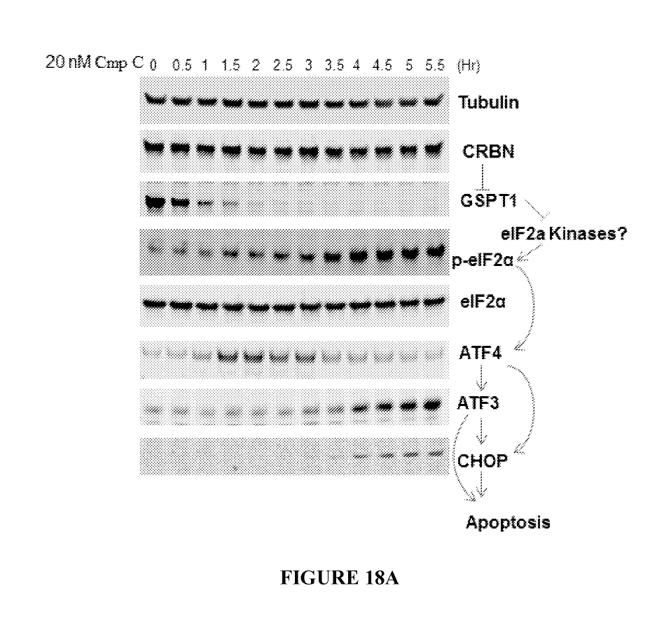

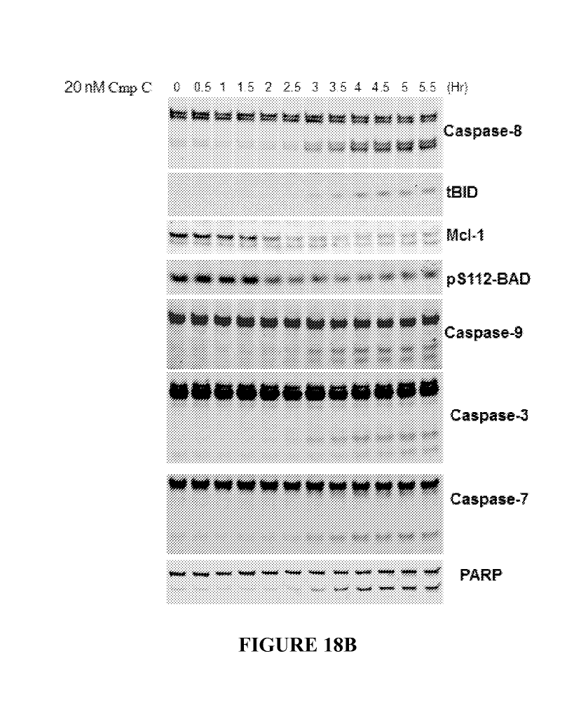

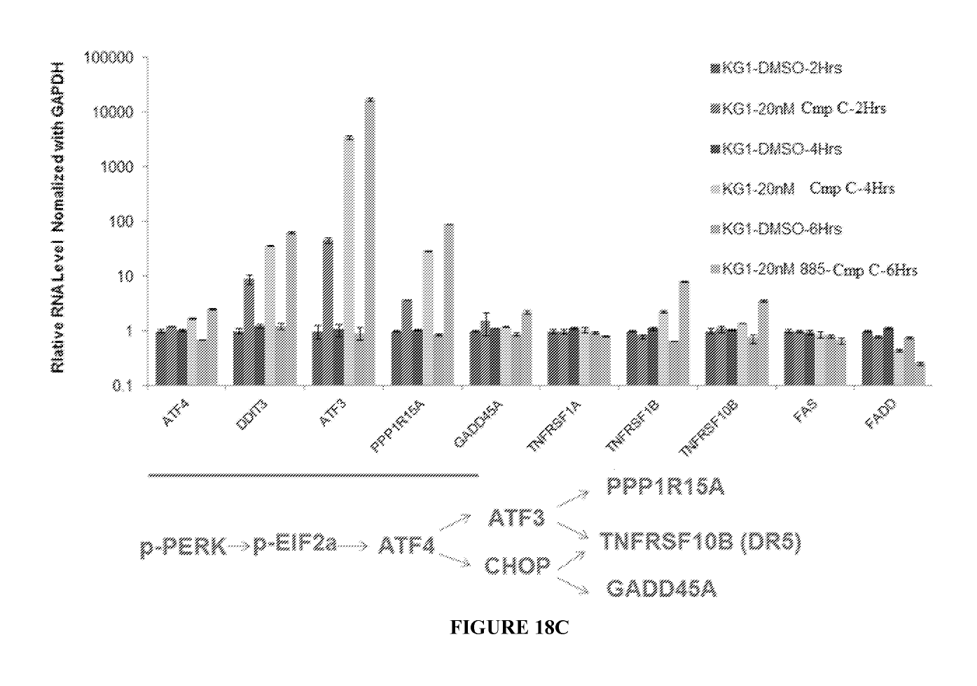

The cellular effect of Compound C-induced degradation of GSPT1 was further studied in KG1 cells. Cells were treated with DMSO alone or 20 nM Compound C. The expression levels of various cellular components along UPR or apoptosis pathways were measured at various time points post treatment. The results indicated that Compound C induced degradation of GSPT1. Similarly, Compound C increased the expression of ATF-4 and its downstream target ATF-3. The levels of pEIF2α, DDIT3, cleaved Caspase-3, and cleaved PARP also increased, which suggested the onset of apoptosis. Representative results of this study are shown in FIG. 18A and FIG. 18B.

FIG. 18A shows that Compound C induces degradation of GSPT1, and that the protein levels of pEIF2α, ATF4, ATF3, and CHOP (DDIT3) increase in response to Compound C treatment. FIG. 18B shows that the levels of cleaved Caspase-8, BID, cleaved Caspase-9, cleaved Caspase-3, cleaved Caspase-7, and cleaved PARP increase in response to Compound C treatment, and that the levels of Mcl-1 and pS112-BAD decrease in response to Compound C treatment.

The mRNA level was quantified as shown in FIG. 18C, demonstrating Compound C-induced expression of ATF4, ATF3, DDIT3, PPP1R15A, GADD45A, TNFRSF1B, and TNFRSF10B, components along the PERK/EIF2a/ATF4 pathway in KG1 cells.

6.20 Compound C Induces UPR in Human Acute Myeloblastic Leukemia Cell Line KG1.

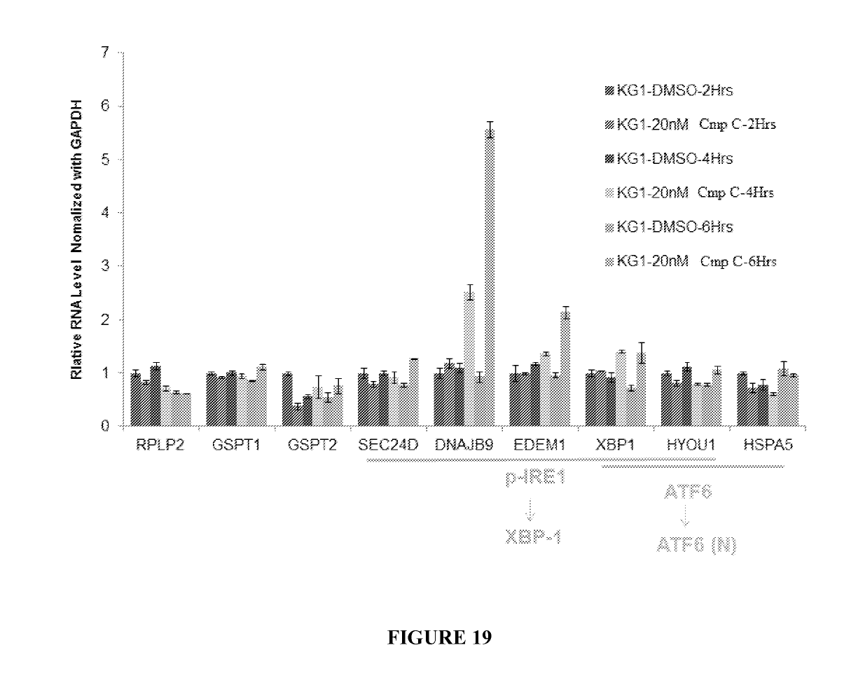

The mechanism of Compound C-activated XBP1 and ATF6 pathways of UPR was studied in KG1 cells. Cells were treated with DMSO or 20 nM Compound C for 2, 4, or 6 hours. The RNA level of variant cellular components along the XBP1 and ATF6 pathways was measured and normalized with GAPDH. As shown in FIG. 19, Compound C induces expression of components along the XBP1 pathway (such as SEC24D, DNAJB9, EDEM1, and XBP1) and components along the ATF6 pathway (such as XBP1).

6.21 Response to Compound C Treatment in Normal Peripheral Blood Mononuclear Cell (PBMC)

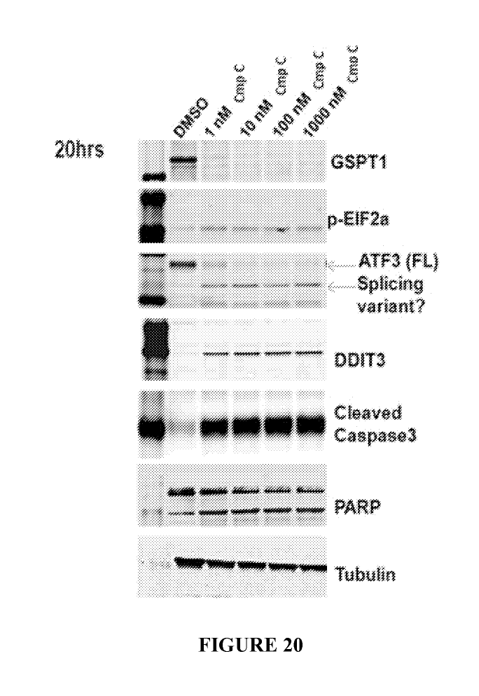

The response of PBMC to Compound C treatment was monitored by measuring the expression of GSPT1, ATF3, DDIT3, and downstream apoptosis indicators in PBMC. PBMCs were treated with 1 nM, 10 nM, 100 nM, or 1000 nM of Compound C for 20 hours. As shown in FIG. 20, Compound C decreases the expression of GSPT1, but increases the level of p-EIF2α, ATF3 (likely in a splicing variant) and DDIT3, which consequently activate caspase 3 by increasing cleaved Caspase-3. The cleaved Caspase-3 then inactivates PARP by cleaving PARP and induces apoptosis. Thus, GSPT1, ATF3, DDIT3, cleaved Caspase-3, and cleaved PARP can serve as biomarkers predicting the toxicity of Compound C.

6.22 Prediction of Sensitivity and Resistance to Compound C Analogues in Different Cancer Cell Lines

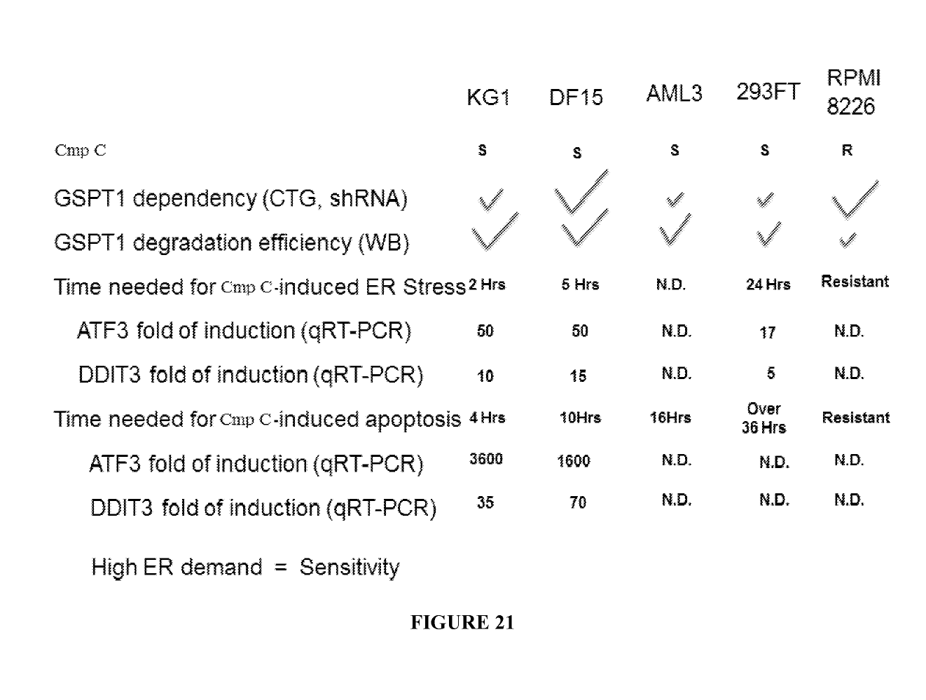

Different cancer cell lines exhibit various sensitivities to the treatment of Compound C. The GSPT1 dependency was shown by GSPT1-specific shRNA knockdown experiment. The GSPT1 degradation efficiency was shown by Western Blot. The induction of ATF3 or DDIT3 was measured by quantitative RT-PCR. As shown in FIG. 21, Compound C-induced ER stress precedes Compound C-induced apoptosis. Time needed for Compound C-induced ER stress or Compound C-induced apoptosis was summarized. Among all the cancer cell lines tested herein, RPMI-8226 is resistant to Compound C-induced ER stress and apoptosis. The other cells, such as KG1, DF15, AML3, and 293FT, exhibit different levels of sensitivity to Compound C. High ER demand may contribute to the sensitivity of certain cancer cells to the treatment of Compound C.

From the foregoing, it will be appreciated that, although specific embodiments have been described herein for the purpose of illustration, various modifications may be made without deviating from the spirit and scope of what is provided herein. All of the references referred to above are incorporated herein by reference in their entireties.

What is claimed is:

1. A method of identifying a subject having cancer who is likely to be responsive to a treatment compound, comprising:
(a) administering the treatment compound to the subject having the cancer;
(b) obtaining a sample from the subject;
(c) determining the level of a biomarker in the sample from the subject; and
(d) diagnosing the subject as being likely to be responsive to the treatment compound if the level of the biomarker in the sample of the subject changes as compared to a reference level of the biomarker;
wherein the treatment compound is a compound of Formula I: 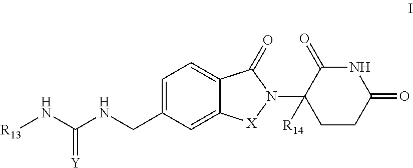
or a pharmaceutically acceptable salt, solvate, stereoisomer, isotopologue, prodrug, hydrate, co-crystal, clathrate, or a polymorph thereof, wherein:
X is CH₂ or C═O;
Y is O or S;
R¹³ is: (C₁-C₁₀)alkyl; (C₁-C₁₀)alkoxy; or 5 to 10 membered aryl or heteroaryl, optionally substituted with one or more of:
halogen; cyano; (C₁-C₆)alkylenedioxy; (C₁-C₆)alkoxy, itself optionally substituted with one or more halogen; (C₁-C₆)alkyl, itself optionally substituted with one or more halogen; or (C₁-C₆)alkylthio, itself optionally substituted with one or more halogen; and
R¹⁴ is H or (C₁-C₆)alkyl.

2. A method of identifying a subject having cancer who is likely to be responsive to a treatment compound, comprising:
(a) obtaining a sample from the subject having the cancer;
(b) administering the treatment compound to the sample from the subject having the cancer;
(c) determining the level of a biomarker in the sample from the subject; and
(d) diagnosing the subject as being likely to be responsive to the treatment compound if the level of the biomarker in the sample of the subject changes as compared to a reference level of the biomarker;
wherein the treatment compound is a compound of Formula I: 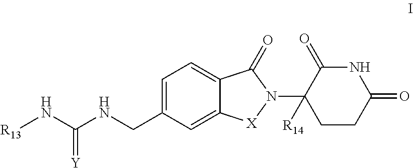
or a pharmaceutically acceptable salt, solvate, stereoisomer, isotopologue, prodrug, hydrate, co-crystal, clathrate, or a polymorph thereof, wherein:
X is CH₂ or C═O;
Y is O or S;
R¹³ is: (C₁-C₁₀)alkyl; (C₁-C₁₀)alkoxy; or 5 to 10 membered aryl or heteroaryl, optionally substituted with one or more of:
halogen; cyano; (C₁-C₆)alkylenedioxy; (C₁-C₆)alkoxy, itself optionally substituted with one or more halogen; (C₁-C₆)alkyl, itself optionally substituted with one or more halogen; or (C₁-C₆)alkylthio, itself optionally substituted with one or more halogen; and
R¹⁴ is H or (C₁-C₆)alkyl.

3. The method of claim 1 or claim 2, wherein the change in the level of the biomarker in the sample of the subject is an increase compared to the reference level of the biomarker.

4. The method of claim 1 or claim 2, wherein the change in the level of the biomarker in the sample of the subject is a decrease compared to the reference level of the biomarker.

5. A method of treating cancer, comprising:
(a) obtaining a sample from a subject having the cancer;
(b) determining the level of a biomarker in the sample from the subject;
(c) diagnosing the subject as being likely to be responsive to a treatment compound if the level of the biomarker in the sample of the subject changes as compared to a reference level of the biomarker; and
(d) administering a therapeutically effective amount of the treatment compound to the subject diagnosed as being likely to be responsive to the treatment compound;
wherein the treatment compound is a compound of Formula I: 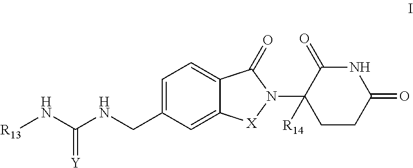
or a pharmaceutically acceptable salt, solvate, stereoisomer, isotopologue, prodrug, hydrate, co-crystal, clathrate, or a polymorph thereof, wherein:
X is CH₂ or C═O;
Y is O or S;
R¹³ is: (C₁-C₁₀)alkyl; (C₁-C₁₀)alkoxy; or 5 to 10 membered aryl or heteroaryl, optionally substituted with one or more of:
halogen; cyano; (C₁-C₆)alkylenedioxy; (C₁-C₆)alkoxy, itself optionally substituted with one or more halogen; (C₁-C₆)alkyl, itself optionally substituted with one or more halogen; or (C₁-C₆)alkylthio, itself optionally substituted with one or more halogen; and
R¹⁴ is H or (C₁-C₆)alkyl.

6. The method of claim 5, wherein the change in the level of the biomarker in the sample of the subject is an increase compared to the reference level of the biomarker.

7. The method of claim 5, wherein the change in the level of the biomarker in the sample of the subject is a decrease compared to the reference level of the biomarker.

8. A method of predicting the responsiveness of a subject having or suspected of having cancer to a treatment compound, comprising:
(a) administering the treatment compound to the subject having the cancer;
(b) obtaining a sample from the subject;
(c) determining the level of a biomarker in the sample from the subject;
(d) diagnosing the subject as being likely to be responsive to treating the cancer with the treatment compound if the level of the biomarker in the sample changes as compared to the level of the biomarker obtained from a reference sample;
wherein the treatment compound is a compound of Formula I: 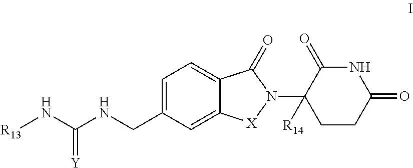
or a pharmaceutically acceptable salt, solvate, stereoisomer, isotopologue, prodrug, hydrate, co-crystal, clathrate, or a polymorph thereof, wherein:
X is CH₂ or C═O;
Y is O or S;
R¹³ is: (C₁-C₁₀)alkyl; (C₁-C₁₀)alkoxy; or 5 to 10 membered aryl or heteroaryl, optionally substituted with one or more of:
halogen; cyano; (C₁-C₆)alkylenedioxy; (C₁-C₆)alkoxy, itself optionally substituted with one or more halogen; (C₁-C₆)alkyl, itself optionally substituted with one or more halogen; or (C₁-C₆)alkylthio, itself optionally substituted with one or more halogen; and
R¹⁴ is H or (C₁-C₆)alkyl.

9. A method of predicting the responsiveness of a subject having or suspected of having cancer to a treatment compound, comprising:
(a) obtaining a sample from the subject having the cancer;
(b) administering the treatment compound to the sample from the subject having the cancer;
(c) determining the level of a biomarker in the sample from the subject;
(d) diagnosing the subject as being likely to be responsive to treating the cancer with the treatment compound if the level of the biomarker in the sample changes as compared to the level of the biomarker obtained from a reference sample;
wherein the treatment compound is a compound of Formula I: 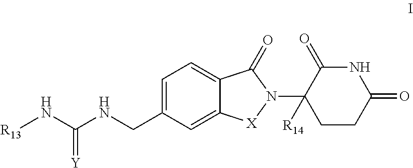
or a pharmaceutically acceptable salt, solvate, stereoisomer, isotopologue, prodrug, hydrate, co-crystal, clathrate, or a polymorph thereof, wherein:
X is CH₂ or C═O;
Y is O or S;
R¹³ is: (C₁-C₁₀)alkyl; (C₁-C₁₀)alkoxy; or 5 to 10 membered aryl or heteroaryl, optionally substituted with one or more of:
halogen; cyano; (C₁-C₆)alkylenedioxy; (C₁-C₆)alkoxy, itself optionally substituted with one or more halogen; (C₁-C₆)alkyl, itself optionally substituted with one or more halogen; or (C₁-C₆)alkylthio, itself optionally substituted with one or more halogen; and
R¹⁴ is H or (C₁-C₆)alkyl.

10. The method of claim 8 or claim 9, wherein the level of the biomarker in the sample is higher than the level of the biomarker obtained from the reference sample.

11. The method of claim 8 or claim 9, wherein the level of the biomarker in the sample is lower than the level of the biomarker obtained from the reference sample.

12. A method of monitoring the efficacy of a treatment compound in treating a subject having cancer, comprising:
(a) administering the treatment compound to the subject having the cancer;
(b) obtaining a sample from the subject having the cancer;
(c) determining the level of a biomarker in the sample from the subject;
(d) comparing the level of the biomarker in the sample with the level of the biomarker obtained from a reference sample, wherein a change in the level as compared to the reference is indicative of the efficacy of the treatment compound in treating the cancer in the subject;
wherein the treatment compound is a compound of Formula I: 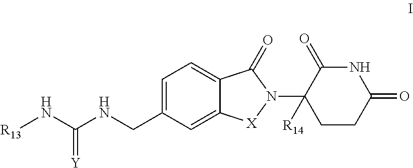
or a pharmaceutically acceptable salt, solvate, stereoisomer, isotopologue, prodrug, hydrate, co-crystal, clathrate, or a polymorph thereof, wherein:
X is CH₂ or C═O;
Y is O or S;
R¹³ is: (C₁-C₁₀)alkyl; (C₁-C₁₀)alkoxy; or 5 to 10 membered aryl or heteroaryl, optionally substituted with one or more of:
halogen; cyano; (C₁-C₆)alkylenedioxy; (C₁-C₆)alkoxy, itself optionally substituted with one or more halogen; (C₁-C₆)alkyl, itself optionally substituted with one or more halogen; or (C₁-C₆)alkylthio, itself optionally substituted with one or more halogen; and
R¹⁴ is H or (C₁-C₆)alkyl.

13. The method of claim 12, wherein an increased level as compared to the reference is indicative of the efficacy of the treatment compound in treating the cancer in the subject.

14. The method of claim 12, wherein a decreased level as compared to the reference is indicative of the efficacy of the treatment compound in treating the cancer in the subject.

15. The method of any one of claims 1-14, wherein the biomarker is a protein that is directly or indirectly affected by CRBN.

16. The method of any one of claims 5-7, further comprising administering a therapeutically effective amount of a second active agent or a support care therapy.

17. The method of claim 16, wherein the second active agent is a hematopoietic growth factor, cytokine, anti-cancer agent, antibiotic, cox-2 inhibitor, immunomodulatory agent, immunosuppressive agent, corticosteroid, therapeutic antibody that specifically binds to a cancer antigen or a pharmacologically active mutant, or derivative thereof.

18. The method of any one of claims 1 to 17, wherein the reference is prepared by using a control sample obtained from the subject prior to administering the treatment compound to the subject, and wherein the control sample is from the same source as the sample.

19. The method of any one of claims 1 to 17, wherein the reference is prepared by using a control sample obtained from a healthy subject not having the cancer, and wherein the control sample is from the same source as the sample.

20. The method of any one of claims 1-19, wherein the cancer is multiple myeloma (MM), lymphoma, or leukemia.

21. The method of any one of claims 1-19, wherein the cancer is lymphoma.

22. The method of any one of claims 1-19, wherein the cancer is leukemia.

23. The method of claim 22, wherein the leukemia is chronic lymphocytic leukemia, chronic myelocytic leukemia, acute lymphoblastic leukemia, or acute myeloid leukemia.

24. The method of claim 22, wherein the leukemia is acute myeloid leukemia (AML).

25. The method of any one of claims 22-24, wherein the leukemia is relapsed, refractory or resistant to conventional therapy.

26. The method of any one of claims 1-25, wherein the biomarker is a CRBN-associated protein.

27. The method of any one of claims 1-25, wherein the biomarker has a function in unfolded protein response (UPR).

28. The method of any one of claims 1-25, wherein the biomarker has a function in PERK related signaling pathway.

29. The method of any one of claims 1-25, wherein the biomarker has a function in XBP1 related signaling pathway.

30. The method of any one of claims 1-25, wherein the biomarker has a function in ATF6 related signaling pathway.

31. The method of any one of claims 1-25, wherein the biomarker is an eRF3 family member selected from the group consisting of eRF3a, eRF3b, eRF3c.

32. The method of claim 31, wherein the biomarker is eRF3a, eRF3b, or eRF3c, and wherein the level of the biomarker decreases as compared to a reference.

33. The method of claim 31, wherein the biomarker is eRF3a.

34. The method of claim 31, wherein the biomarker is eRF3b.

35. The method of claim 31, wherein the biomarker is eRF3c.

36. The method of claim 26, wherein the biomarker is selected from the group consisting of ATF4, ATF3 and DDIT3, and wherein the level of the biomarker increases as compared to a reference.

37. The method of claim 36, wherein the biomarker is ATF4.

38. The method of claim 36, wherein the biomarker is ATF3.

39. The method of claim 36, wherein the biomarker is DDIT3.

40. The method of any one of claims 1 to 39, wherein the level of the biomarker is measured by determining the protein level of the biomarker.

41. The method of claim 40, comprising contacting proteins within the sample with a first antibody that immunospecifically binds to the biomarker protein.

42. The method of claim 41, further comprising:
(i) contacting the biomarker protein bound to the first antibody with a second antibody with a detectable label, wherein the second antibody immunospecifically binds to the biomarker protein, and wherein the second antibody immunospecifically binds to a different epitope on the biomarker protein than the first antibody;
(ii) detecting the presence of the second antibody bound to the biomarker protein; and
(iii) determining the amount of the biomarker protein based on the amount of detectable label in the second antibody.

43. The method of claim 41, further comprising:
(i) contacting the biomarker protein bound to the first antibody with a second antibody with a detectable label, wherein the second antibody immunospecifically binds to the first antibody;
(ii) detecting the presence of the second antibody bound to the first antibody; and
(iii) determining the amount of the biomarker protein based on the amount of detectable label in the second antibody.

44. The method of any one of claims 1 to 39, wherein the level of the biomarker is measured by determining the mRNA level of the biomarker.

45. The method of any one of claims 1 to 39, wherein the level of the biomarker is measured by determining the cDNA level of the biomarker.

46. The method of any one of claims 1 to 45, wherein the treatment compound is a compound of Formula I: 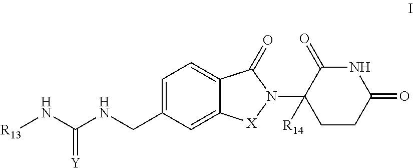
or a pharmaceutically acceptable salt, solvate, stereoisomer, isotopologue, prodrug, hydrate, co-crystal, clathrate, or a polymorph thereof, wherein:
X is CH₂;
Y is O;
R¹³ is 5 to 10 membered aryl or heteroaryl, optionally substituted with one or more of: halogen; cyano; (C₁-C₆)alkylenedioxy; (C₁-C₆)alkoxy, itself optionally substituted with one or more halogen; (C₁-C₆)alkyl, itself optionally substituted with one or more halogen; or (C₁-C₆)alkylthio, itself optionally substituted with one or more halogen; and
R¹⁴ is H.

47. The method of any one of claims 1 to 46, wherein the treatment compound is 1-(3-chloro-4-methylphenyl)-3-((2-(2,6-dioxopiperidin-3-yl)-1-oxoisoindolin-5-yl)methyl)urea 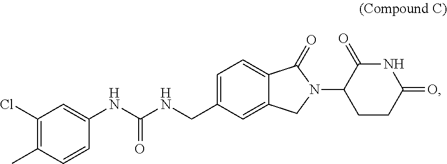 [SMILES: Cc1ccc(NC(=O)NCc2ccc3c(c2)CN(C2CCC(=O)NC2=O)C3=O)cc1Cl]
or a pharmaceutically acceptable salt, solvate, stereoisomer, isotopologue, prodrug, hydrate, co-crystal, clathrate, or a polymorph thereof.
